(19) United States
(12) Patent Application Publication
(10) Pub. No.: US 2026/0124122 A1
(43) Pub. Date: May 7, 2026

(54) QUATERNARY AMMONIUM SILANE AQUEOUS HYDROGEL FORMULATIONS

(71) Applicant: TOPIKOS SCIENTIFIC, INC., Fort Wayne, IN (US)

(72) Inventors: Gary Allred, Wake Forest, NC (US); Lany Liebeskind, Atlanta, GA (US); William R. Cast, Fort Wayne, IN (US); Carl Hilliard, Henderson, NV (US)

(73) Assignee: TOPIKOS SCIENTIFIC, INC., Fort Wayne, IN (US)

(21) Appl. No.: 19/427,735

(22) Filed: Dec. 19, 2025

Related U.S. Application Data
(63) Continuation of application No. PCT/US2024/050279, filed on Oct. 7, 2024.
(60) Provisional application No. 63/543,010, filed on Oct. 6, 2023.
(60) Provisional application No. 63/547,069, filed on Nov. 2, 2023.

(51) Int. Cl. A61K 8/04; A61K 8/34; A61K 8/58; A61K 8/73; A61K 8/86; A61Q 11/00

(52) U.S. Cl. CPC A61K 8/042; A61K 8/345; A61K 8/585; A61K 8/731; A61K 8/86; A61Q 11/00

(57) ABSTRACT

Disclosed herein are quaternary ammonium silane aqueous hydrogel formulations, including pharmaceutically acceptable aqueous hydrogel formulations, that provide high aqueous stability to an ammonium silane active moiety, as well as methods of use and manufacture.

### CROSS-REFERENCE TO RELATED APPLICATIONS

This application is a continuation of International Patent Application No. PCT/US2024/050279, filed in U.S. Receiving Office on Oct. 7, 2024, which claims the benefit of U.S. Provisional Application No. 63/543,010, which was filed on Oct. 6, 2023, and U.S. Provisional Application No. 63/547,069, which was filed on Nov. 2, 2023. The entirety of each of these applications is incorporated by reference for all purposes.

### FIELD OF THE INVENTION

This disclosure provides quaternary ammonium silane formulations with enhanced stability useful for reducing, preventing, or inhibiting the growth and development of opportunistic pathogenic microbial organisms (pathogens). The formulations described herein are useful for treating or preventing harmful microbial infections, including polymicrobial infections such as those found in microbial biofilms, on or in a host such as an animal or human. Furthermore, the formulations described herein can be used in an amount sufficient to (a) inhibit the detachment and dissemination of pathogenic cells from extracellular polymeric substances (EPS) of a biofilm; (b) cause a regression of destabilizing overgrowth of pathogenic cells and symbiotic enablers in harmful biofilms; and, (c) inhibit or prevent the attachment and formation of new biofilms by dispersed cells.

### BACKGROUND OF THE INVENTION

Biofilms are structured colonies of various microorganisms, such as bacteria, fungi, and viruses, that live inside of a protective exopolysaccharide matrix responsible for the colony's survival. Biofilms are complex surface attached communities of microorganisms held together by self-produced polymer matrices mainly composed of polysaccharides, secreted proteins, and extracellular DNAs (Tremblay et al., (2013). Method to grow Actinobacillus pleuropneumoniae biofilm on a biotic surface. BMC Vet. Res. 9:213). A biofilm can consist of a single microbial species or a combination of different species of bacteria, protozoa, archaea, algae, filamentous fungi, and yeast that strongly attach to each other and to biotic or abiotic surfaces (Raghupathi et al., (2017)), or at air-liquid interface. Synergistic interactions within a multispecies biofilm enhance individual species protection against grazing by a pelagic protozoan (Front. Microbiol. 8:2649. The ability of microorganisms to develop biofilms has been shown to be an adaptable attribute of microbes (Koczan et al., (2011). Cell surface attachment structures contribute to biofilm formation and xylem colonization by Erwinia amylovora. Appl. Environ. Microbiol. 77, 7031-7039). The formation of biofilm appears to be an evolutionarily-adapted phenomenon that provides microorganisms with strengthened survival mechanisms when compared with individual planktonic cells (Dang and Lovell (2016). Microbial surface colonization and biofilm development in marine environments. Microbiol. Mol. Biol. Rev. 80, 91-138), including enhanced ability to grow in oligotrophic environments (Bowden and Li (1997). Nutritional influences on biofilm development. Adv. Dent. Res. 11, 81-99.), greater access to nutritional resources (Dang and Lovell, 2016), improved survival to biocides (Flemming et al. (2016). Biofilms: an emergent form of bacterial life. Nat. Rev. Microbiol. 14, 563-575), enhanced organism productivity and interactions (Roder et al. (2018). Enhanced bacterial mutualism through an evolved biofilm phenotype. ISME J. 12, 2608-2618), as well as greater environmental stability (Dang and Lovell, 2016). It is readily apparent that biofilms provide novel mechanisms of protection for communities of bacterial species under adverse environmental conditions.

Biofilm formation is a complex process and can be described in five main phases: (i) reversible attachment phase, where microbes such as bacteria non-specifically attach to surfaces; (ii) irreversible attachment phase, which involves interaction between microbial cells and a surface using, for example, bacterial adhesins such as fimbriae and lipopolysaccharide (LPS); (iii) production of extracellular polymeric substances (EPS) by the resident microbial cells; (iv) biofilm maturation phase, in which microbe cells synthesize and release signaling molecules to sense the presence of each other, conducing to the formation of microcolony and maturation of biofilms; and (v) dispersal/detachment phase, where the microbial cells depart biofilms and comeback to independent planktonic lifestyle (Muhammad et al., Beyond Risk: Bacterial Biofilms and Their Regulating Approaches. Front. Microbiol. 21 May 2020).

The oral cavity is home to over 700 species of microbes, including bacteria, fungi, viruses, and protozoa. Together they form a complex biological system called the oral microbiome (de Jongh, de Vries et al. 2023). Most of these microbes convert from free floating planktonic form into a sessile form to integrate into a surface attached, polymicrobial community encased in a self-produced extracellular polymeric substance (EPS) or “biofilm.” Individual species within a biofilm form microcolonies that interact with each other using sophisticated communication methods called “quorum-sensing” (Huang R 2011). These colonies cooperate and compete to colonize dental and periodontal tissues (Marcano R, Rojo M et al. 2021).

Dental biofilms are called “plaque.” Bacteria account for the majority of the microbes living in plaque (Zhang Y, Wang X et al. 2018). Some of these bacteria (e.g. Streptococcus mutans (S. mutans), non-mutans Streptococci, Lactobacillus, Bifidobacterium, and Actinomyces) metabolize carbohydrates to produce organic acids, primarily lactic acid, which “accumulate in the fluid phase of the plaque biofilm, demineralizing the surface layer of the tooth” (cavities or caries) (Rathee M and Sapra A 2023). Almost all adults are affected by caries (Peterson P E, Bourgeois D et al. 2023). Caries remains an unresolved public health problem (Kim D 2020) with an estimated cost over $120 billion per year in the US alone (Zhang P 2022).

Plaque forms on the surface of teeth and gums in a sequential process starting with “pioneer” planktonic microbes. Pioneer microbes are a distinct group of planktonic bacteria having adherent abilities (Murata R M, Branco-de-Almeida L S et al. 2010). They form a foundational layer, known as the “acquired pellicle,” on the gingival margin between the teeth and gums (dentogingival junction) (Pollanen, Laine et al. 2012). These organisms, particularly S. mutans, synthesize extracellular glucans from sucrose to attach to the acquired pellicle (Susmitha A, Bajaj H and K. 2021). Other planktonic bacteria join by co-aggregating with the pioneer microbes through adhesion-receptor interactions. A common opportunistic fungal pathogen, Candida albicans, is frequently found with high levels of S. mutans in plaque. This synergistic combination increases the volume of the EPS, limits diffusion, contributes to stability, and provides enhanced virulence (Falsetta M L, Klein M I et al. 2014). Such bacteria-fungus combination may also provide fungal reservoirs for Candida and viruses that cause other diseases (Falsetta M L, Klein M I et al. 2014, Von Borowski and Trentin 2021). Quorum sensing determines when enough microbes have been aggregated on the acquired pellicle to form a plaque and up regulate from planktonic to a sessile form of life. In upregulating, the individual microbes genetically adapt to perform specialized roles as members of the biofilm community.

Co-aggregation of species is the foundation for the interactions of microbes within the biofilm. Fusobacteria play a central role as physical bridges that mediate co-aggregation and facilitate gram-negative anaerobes aggregation. Fusobacterium nucleatum guarantees, directly or through Treponema denticola, the adhesion of P. gingivalis (Marcano R, Rojo M et al. 2021). P. gingivalis can produce several virulence factors which evade host defenses and can cause damage to the dental tissues and bone (Lamont R, Koo and G. 2018). However, no single microbial species (nor even a select few species) is sufficiently pathogenic on its own to cause disease (Curtis M, Hajishengallis G et al. 2011). According to the “keystone pathogen hypothesis,” key pathogens, such as P. gingivalis, do not directly cause disease but rather synergistically elevate the pathogenic potential of the entire plaque community through interactive quorum sensing communication with accessory pathogens, such as S. gordonii, and disruption of immune surveillance (Olsen, Lambris and Hajishengallis 2017). Other bacterial species in the plaque community, particularly members of the genera Veillonella, Fusobacterium and Prevotella, then trigger a destructive host immune inflammation response.

The balance between the sessile cells in the plaque community and with the host play an important role in human biology, the maintenance of health and the development of disease (Curtis M, Hajishengallis G et al. 2011). A predominant community of gram-positive aerobic cells holds in check the pathogenic gram-negative anaerobic supragingival cells resulting in a homeostatic state with the host. A change from a balanced community to one dominated by predominately gram-negative cells (dysbiosis state) leads to pathogenesis (Hou, Wu et al. 2022, Hrncir 2022).

Periodontal disease (gingivitis and periodontitis) is a pathogenic condition associated with dysbiosis of bacterial communities in plaque. (Patini R., Staderini E. et al. 2018). Many bacterial species can make up plaque, however only a handful of bacterial cells are associated with the progression of periodontal disease. These are the gram-negative, anaerobic, proteolytic bacteria Porphyromonas gingivalis (P. gingivalis), Treponema denticola, and Tannerella forsythia and Fusobacterium nucleatum subspecies (Tuominen and Rautava 2021). The biofilm population becomes predominately anaerobic (dysbiosis) within days of co-aggregation of the red complex bacteria with the pioneer microbes (Kolenbrander 2000).

Strong associations have been found between periodontitis and several systemic conditions (Kirst M, Li E et al. 2014). Periodontal pathogens and their metabolic subproducts may enter the bloodstream through inflamed periodontal tissue and lymph vessels or via saliva through dental cleaning or from daily routines like tooth brushing (Kallio 2014, Cardiology. 2023). Some argue that dispersal is the starting point of systemic infections with the release of bacteria into the host (Wang R, Khan B A et al. 2011). Patients suffering from periodontitis have a 2-5 times higher risk of acquiring any cancer compared to healthy individuals (Tuominen and Rautava 2021). Oral cavity-linked bacterial microbes have been linked to endocarditis, aspiration pneumonia, osteomyelitis, diabetes, Alzheimer's, rheumatoid arthritis, and cardiovascular disease. Id. Localized bacterium-fungus interactions found in plaque are associated with other polymicrobial infections and systemic complications at various sites in humans (Falsetta M L, Klein M I et al. 2014).

Restoration of homeostasis between the microbial community and its host is the objective in treatment of periodontal disease (Lasserre J F, Brecx M C and S. 2018). The goal is not to sterilize the oral cavity but to control the subgingival microbes to prevent dysbiosis and maintain homeostasis between the microbial community and its host (Olsen, Lambris and Hajishengallis 2017).

Relatedly, chronic non-healing wounds have been heralded as a silent epidemic, causing significant morbidity and mortality especially in elderly, diabetic, and obese populations (Kalan L, Loesche M, Hodkinson B P, Heilmann K, Ruthel G, Gardner S E, Grice E A. 2016. Redefining the chronic-wound microbiome: fungal communities are prevalent, dynamic, and associated with delayed healing. mBio 7 (5): e01058-16. doi: 10.1128/mBio.01058-1). Chronic wounds are a major healthcare burden, with huge public health and economic impact. Chronic wounds start as a cutaneous wound, which results from injury, surgical incisions, burn wounds or any cutaneous insult which alters the anatomy and function of the epithelial lining of the skin. Although most cutaneous wounds heal in a defined manner, chronic wounds fail to heal and instead get arrested in a prolonged inflammatory to proliferative phase, leading to a persistent, non-healing state that defines chronic wounds (Attinger C., Wolcott R. Clinically Addressing Biofilm in Chronic Wounds. Adv. Wound Care. 2012; 1:127-132). Chronic wounds are often associated with underlying predisposing factors such as diabetes, hypertension, obesity, malignancies, peripheral vascular disease, prolonged immobilization and advanced age (Mustoe T. Understanding chronic wounds: A unifying hypothesis on their pathogenesis and implications for therapy. Am. J. Surg. 2004; 187: S65-S70). Chronic wounds include pressure ulcers, venous leg ulcers, diabetic foot and leg ulcers, partial and full thickness wounds, post-surgical wound, first and second degree burns and grafted and donor sites. Common features shared by these wounds include prolonged or excessive inflammation, persistent infections, formation of antimicrobial resistant biofilms, and the inability of the host immune system to clear the wound. The majority of wounds are initially colonized with commensal flora, which plays a critical role in initiating various inflammatory and signaling events in the wound bed. However, the chronic wound biofilm state results from pathogenic microbes and is most often polymicrobial (Bowler P. G., Duerden B. I., Armstrong D. G. Wound microbiology and associated approaches to wound management. Clin. Microbiol. Rev. 2001; 14:244-269). Most common bacteria implicated in chronic wound biofilms include Staphylococcus aureus, Pseudomonas aeruginosa and β-haemolytic Streptococci; other bacteria include Enterococcus spp, Klebsiella pneumoniae, Acinetobacter baumanii and Enterobacter spp. (ESKAPE pathogens), coagulase-negative Staphylococci and Proteus spp. Unfortunately, conventional antimicrobial strategies are generally insufficient to counteract these biofilms which are increasingly resistant to treatment by chemical agents (Alves, P., Barreto, R., Barrois, B., Gryson, L., Meaume, S., Monstrey, S., Update on the role of antiseptics in the management of chronic wounds with critical colonization and/or biofilm, Int. Wound J., v.18 (3): 2021 June, PMC8244012).

Although various novel treatment options are available, these show varying degrees of efficacy and the eradication of biofilms, including polymicrobial biofilms, in diseases still remains enigmatic. This lack of efficacy is especially troubling in light of the increased incidence of biofilm associated infections. Accordingly, there remains a strong need for safe and effective compositions to treat a range of microbial biofilms composed of mixed pathogens.

### SUMMARY OF THE INVENTION

Disclosed herein are quaternary ammonium silane aqueous hydrogel formulations, including pharmaceutically acceptable aqueous hydrogel formulations, that provide high stability to an ammonium silane active moiety, as well as methods of use and manufacture. The aqueous hydrogel formulations provided herein comprise:
a) a quaternary ammonium silane compound selected from the group consisting of Formula A-P and Compounds I-LIV;
b) a non-ionic gelling agent;
c) a non-ionic adhesion agent;
d) a non-ionic wetting agent;
e) optionally a non-ionic osmotic agent;
f) optionally one or more additional agents as described herein; and,
g) an aqueous solution.

The aqueous hydrogel formulations described herein provide for enhanced stability of the quaternary ammonium silane active moiety and extend residence time when applied topically, including in moist environments such as on a wound or within an oral cavity, for example, the mouth, teeth, or gums. Importantly, it has been found that the compounds described herein (Formula A-P and Compounds I-LIV), when used in the hydrogel formulations described herein, completely hydrolyze during formation of the hydrogel formulation, typically within 30 to 60 min, resulting in i) a cationic trihydroxy quaternary ammonium silane moiety (“trihydroxy-QAS cation”); ii) an anionic counterion; and iii) a smaller hydroxyl-containing product derived upon the hydrolysis from the side chains attached to the oxygens of the Si—O moiety of the compounds described herein (see, e.g., FIG. 1; FIGS. 2-5):

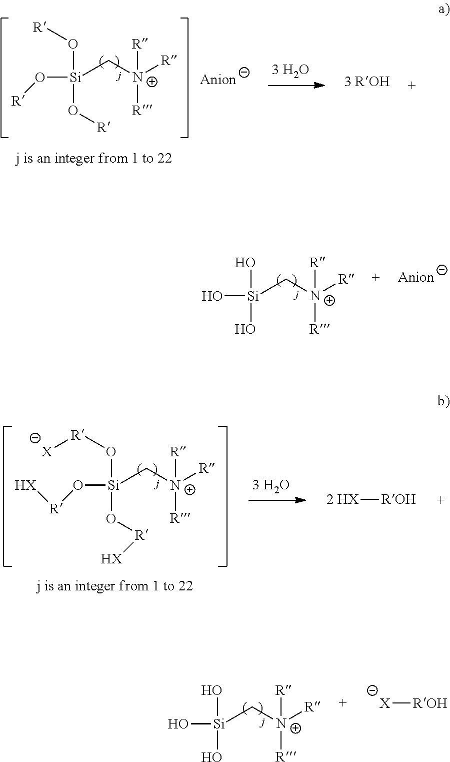

In certain embodiments, the anionic counterion and smaller hydroxyl-containing product act as an agent to additionally stabilize the active trihydroxy quaternary ammonium silane (i.e., the trihydroxy-QAS cation and anion of Formula X-A) from self-polymerization and undesired precipitation within the hydrogel. The resultant trihydroxy-QAS cation, anionic counterion, and a smaller hydroxyl-containing product derived from the hydrolysis of a compound described herein in combination with the non-ionic ingredients of the hydrogel (as discussed further below) results in the reduced polymerization of the silane within the hydrogel, enhancing the aqueous stability of the trihydroxy-QAS. While not being bound to any theory, it is believed that the hydrogel formulation resulting from the in-situ hydrolysis of the compounds described herein as a component of the hydrogel formulation advantageously differs from any formulation that could be formed by mixing the individual hydrolysis products provided separately. This is due to the hydrolysis of a compound selected from Formulas A-P or Compounds I-LIV in the hydrogel proceeding over a short period of time which is enough to avoid any local overconcentrating of hydroxy silicon-containing species which could otherwise result in self-polymerization and due to sufficient time for the hydroxyl-containing compound derived from the side chain of the compound to be hydrolyzed to develop its stabilizing effect.

One significant beneficial effect of reduced polymerization of the trihydroxy-QAS is that it remains available (unpolymerized/unprecipitated) within the hydrogel to bind to surface tissues and to microbial membranes and exert an effective antimicrobial effect. An additional beneficial effect of the reduced polymerization of the trihydroxy-QAS is a significantly increased concentration of a dissolved anti-microbial trihydroxy-QAS residing in the hydrogel, wherein the dissolved trihydroxy-QAS can be sustainably released to the site to be treated over prolonged periods of time to provide the desired topical antimicrobial effect. This increase in the concentration of an active dissolved trihydroxy-QAS allows one to reduce the load of a compound of the invention in the hydrogel formulation if needed, reduce the amount of applied hydrogel formulation, and reduce frequency of applying the hydrogel formulation which simplifies and increase convenience of using the hydrogel formulations described herein. This enhanced stability also allows the anti-microbial trihydroxy-QAS to be used in an aqueous formulation, thus drastically expanding the potential uses of the active moiety in anti-microbial applications. This long-term stabilization is especially useful in treating, inhibiting, and/or preventing microbial growth during long-term, multi-day, -week, or -month applications where aqueous formulations may be preferred, for example in an oral cavity or wound. Accordingly, in certain embodiments, the aqueous ammonium silane hydrogel formulations described herein are stable for at least one month, at least 2 months, at least 3 months, at least 6 months, at least 6 months and greater than 6 months, for example, up to and exceeding 1 year (see, e.g., Examples 11 and 12). Furthermore, it is believed that the dissolved trihydroxy-QAS in the hydrogel bonds to tissue surfaces, thus concentrating the trihydroxy-QAS locally and rendering it non-systemic, non-leaching, and potentially more durable on living tissues, thus increasing therapeutic efficacy.

In some embodiments, the quaternary ammonium silane compound contained within the hydrogel formulation possesses a non-halidic counterion (e.g., Compound XIII or Compound XIX). It has been discovered that within certain aqueous media, the use of a non-halidic counterion further stabilizes the trihydroxy-QAS and the rate of disadvantageous polymerization is slowed or significantly reduced, which is beneficial for the water-stability of the active trihydroxy-QAS within the aqueous hydrogel. Furthermore, it has been discovered that the selection of appropriate formulation ingredients can further enhance the stability, and thus the efficacy, of the trihydroxy-QAS.

In certain embodiments, the formulations mechanically disrupt cell wall membranes (causing lysis) of both bacterial and fungal sessile and planktonic pathogens on contact, thereby re-enforcing the outside of the gel barrier against pathogen intrusion, reducing any contamination within the gel, and incapacitating proximate pathogenic biofilms and ambient organisms that may impede wound healing or assist in reestablishing microbial biofilm colonies, for example in plaque or other oral disorders. In certain embodiments, the quaternary ammonium silane hydrogel formulations described herein help to provide a moist environment which is conducive to wound healing, promotes autolytic debridement, and provides a protective barrier against intrusion by outside pathogens.

In one aspect, provided herein is a stable quaternary ammonium silane aqueous hydrogel formulation comprising:
a) a compound selected from the group consisting of Formula A-P and Compounds I-LIV, wherein the compound is present in the hydrogel formulation at a concentration that results in a trihydroxy-QAS cation present in the hydrogel at between about 0.1% and 5.0% (weight/weight) (w/w) when the compound is fully hydrolyzed, wherein the trihydroxy-QAS cation is of formula

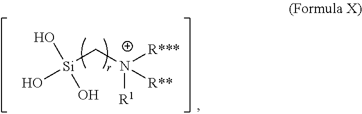

wherein
   r is 1, 2, 3, or 4;
   R¹ is C₆-C₂₂alkyl or C₆-C₂₂ alkanoyl;
   R** and R*** are independently selected from the group consisting of:
   i. C₁-C₄ alkyl; and
   ii. C₆-C₂₂alkyl or C₂-C₂₂alkanoyl;
b) between about 0.25% and about 3.0% (w/w) of a non-ionic gelling agent;
c) between about 0.025% and about 0.5% (w/w) of a non-ionic adhesion agent;
d) between about 0.5% and about 5.0% (w/w) of a non-ionic wetting agent;
e) optionally between about 0.5% and about 7.5% (w/w) of a non-ionic osmotic agent; and,
f) optionally one or more additional agents as described further herein.

In some embodiments, the trihydroxy-QAS cation is of structure:

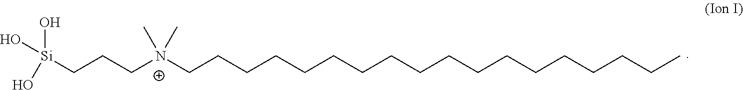 [SMILES: CCCCCCCCCCCCCCCCCC[N+](C)(C)CCC[Si](O)(O)O]

In some embodiments, the non-ionic gelling agent lacks a carboxylic acid group. In some embodiments, the non-ionic wetting agent lacks a carboxylic acid group. In some embodiments, both the non-ionic gelling agent and the non-ionic wetting agent lack a carboxylic acid group. In some embodiments, the hydrogel further comprises a buffer. In some embodiments, the hydrogel further comprises a pH adjusting agent, for example, but not limited to methanesulfonic acid. In some embodiments, the aqueous hydrogel formulation has a pH between about 5 and about 8. In some embodiments, the aqueous hydrogel formulation has a pH between about 5.5 and about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX.

As provided herein, the hydrogel formulation comprises a quaternary ammonium silane selected from Formula A-P and Compounds I-LIV, or a combination thereof. In some embodiments, the hydrogel formulation comprises between about 0.1% and about 5.0% (w/w) of a trihydroxy-QAS cation of Formula X that is derived from the complete hydrolysis of a compound in the hydrogel selected from the group consisting of Formula A-P and Compounds I-LIV, or a combination thereof. In some embodiments, the hydrogel formulation comprises between about 0.25% and about 3.0% (w/w) of a trihydroxy-QAS cation of Formula X that is derived from the complete hydrolysis of a compound in the hydrogel selected from the group consisting of Formula A-P and Compounds I-LIV, or a combination thereof. In some embodiments, the hydrogel formulation comprises between about 0.25% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof. In some embodiments, the quaternary ammonium silane hydrogel formulation comprises between about 0.5% and about 0.75% (w/w) of a trihydroxy-QAS cation of Formula X that is derived from the complete hydrolysis of a compound in the hydrogel selected from the group consisting of Formula A-P and Compounds I-LIV, or a combination thereof. In some embodiments, the quaternary ammonium silane hydrogel formulation comprises about 0.5% (w/w) of a trihydroxy-QAS cation of Formula X that is derived from the complete hydrolysis of a compound in the hydrogel selected from the group consisting of Formula A-P and Compounds I-LIV, or a combination thereof. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

As provided herein, the hydrogel formulation comprises a non-ionic gelling agent. In some embodiments, the non-ionic gelling agent lacks a carboxylic acid group. In some embodiments, the hydrogel formulation comprises a non-ionic gelling agent in the amount of between about 0.5% and about 3.0% (w/w). The selection of the gelling agent assists in ensuring that the trihydroxy-QAS remains in solution. As provided herein, it has been discovered that non-ionic gelling agents, and in particular those which lack a carboxylic acid group, are particularly suitable in further stabilizing the quaternary ammonium silane, compared to, for example, ionic or non-ionic gelling agents that contain a carboxylic acid group such as, for example, sodium alginate, sodium carboxymethylcellulose, carbomer copolymer Type A, carbomer copolymer Type B, and xanthum gum, and other anionic or cationic gelling agents (see, e.g., Example 13). Suitable non-ionic gelling agents lacking carboxylic acid groups for use in the hydrogel formulation include, but are not limited to, hydoxypropyl methylcellulose (HPMC; also known as hypromellose), hydroxyethyl cellulose, methyl cellulose, hydroxypropyl cellulose, polydextrose, palmitostearate, methacrylated inulin, and hydroxypropyl guar. In some embodiments, the gelling agent is HPMC. In some embodiments, the gelling agent is HPMC having a viscosity of between about 2600 and 5600 cP at 2% in water (20° C.). In some embodiments, the quaternary ammonium silane hydrogel formulation comprises between about 0.5% and about 3.0% (w/w) of HPMC. In some embodiments, the quaternary ammonium silane hydrogel formulation comprises about 2.0% (w/w) of the non-ionic gelling agent. In some embodiments, the quaternary ammonium silane hydrogel formulation comprises about 2.0% (w/w) HPMC. In some embodiments, the quaternary ammonium silane hydrogel formulation comprises between about 0.5% and about 1.5% (w/w) of the non-ionic gelling agent. In some embodiments, the quaternary ammonium silane hydrogel formulation comprises between about 0.5% and about 1.5% (w/w) of HPMC. In some embodiments, the quaternary ammonium silane hydrogel formulation comprises between about 0.75% and about 1.25% (weight/weight) of the non-ionic gelling agent. In some embodiments, the quaternary ammonium silane hydrogel formulation comprises between about 0.75% and about 1.25% (w/w) HPMC. In some embodiments, the quaternary ammonium silane hydrogel formulation comprises about 1.0% (w/w) of the non-ionic gelling agent. In some embodiments, the quaternary ammonium silane hydrogel formulation comprises about 1.0% (w/w) HPMC. In some embodiments, the quaternary ammonium silane hydrogel formulation comprises about 1.0% (w/w) HPMC having a viscosity of between about 2600 and 5600 cP at 2% in water (20° C.).

As provided herein, the hydrogel formulation comprises a non-ionic adhesion agent. In some embodiments, the hydrogel formulation comprises a non-ionic adhesion agent in the amount of between about 0.025% and about 0.5% (w/w). Suitable non-ionic adhesion agents include, for example, but not limited to, a poloxamer, polyoxypropylene, polyoxyethylene, and copolymers of polyoxypropylene and polyoxyethylene. In some embodiments, the adhesion agent is a poloxamer, for example, poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate or poloxamer 182 dibenzoate. Additional non-ionic adhesion agents suitable for the formulation provided herein include chitosan, dextrin, glyceryl monoleate, and isopropyl myristate. In some embodiments, the adhesion agent is Poloxamer 407. In some embodiments, the quaternary ammonium silane hydrogel formulation comprises between about 0.05% and about 0.25% (w/w) of the adhesion agent. In some embodiments, the quaternary ammonium silane hydrogel formulation comprises between about 0.05 and about 0.25% (w/w) of a poloxamer, for example, poloxamer 407. In some embodiments, the quaternary ammonium silane hydrogel formulation comprises about 0.2% (w/w) of the adhesion agent. In some embodiments, the quaternary ammonium silane hydrogel formulation comprises about 0.2% (w/w) of a poloxamer, for example, poloxamer 407. In some embodiments, the quaternary ammonium silane hydrogel formulation comprises between about 0.075% and about 0.15% (w/w) an adhesion agent. In some embodiments, the quaternary ammonium silane hydrogel formulation comprises between about 0.075% and about 0.15% (w/w) of a poloxamer, for example, poloxamer 407. In some embodiments, the quaternary ammonium silane hydrogel formulation comprises about 0.1% (w/w) of the gelling agent. In some embodiments, the quaternary ammonium silane hydrogel formulation comprises about 0.1% (w/w) of a poloxamer, for example poloxamer 407.

As provided herein, the hydrogel formulation comprises a non-ionic wetting agent. In some embodiments, the non-ionic wetting agent lacks a carboxylic acid group. In some embodiments, the hydrogel formulation comprises a non-ionic wetting agent in the amount of between about 0.5% and about 5.0% (w/w). The selection of the wetting agent assists in ensuring that the quaternary ammonium silane remains in solution. As provided herein, it has been discovered that non-ionic wetting agents, and in particular those that lack a carboxylic acid group, are particularly suitable in further stabilizing the trihydroxy-QAS silane, compared to, for example, ionic or non-ionic wetting agent that contain a carboxylic acid group such as, for example, sodium hyaluronate or anionic or cationic surfactants. In some embodiments, the non-ionic wetting agent is selected from propylene glycol, polypropylene glycol, glycerin, glycerol, glycerol-propylene oxide copolymer, ethylene glycol, polyvinyl alcohol, or combinations thereof. Additional non-ionic wetting agents are described further below. In some embodiments, the quaternary ammonium silane hydrogel formulation comprises between about 1.0% and about 3.0% (w/w) of the non-ionic wetting agent. In some embodiments, the quaternary ammonium silane hydrogel formulation comprises between about 1.0% and about 3.0% (w/w)propylene glycol. In some embodiments, the quaternary ammonium silane hydrogel formulation comprises between about 1.0% and about 3.0% (w/w) glycerin. In some embodiments, the quaternary ammonium silane hydrogel formulation comprises between about 1.5% and about 2.5% (w/w) of the non-ionic wetting agent. In some embodiments, the quaternary ammonium silane hydrogel formulation comprises between about 1.5% and about 2.5% (w/w)propylene glycol. In some embodiments, the quaternary ammonium silane hydrogel formulation comprises between about 1.5% and about 2.5% (w/w) glycerin. In some embodiments, the quaternary ammonium silane hydrogel formulation comprises about 2.0% (w/w) of the wetting agent. In some embodiments, the quaternary ammonium silane hydrogel formulation comprises about 2.0% (w/w)propylene glycol. In some embodiments, the quaternary ammonium silane hydrogel formulation comprises about 2.0% (w/w) glycerin.

As provided herein, the hydrogel formulation optionally comprises a non-ionic osmotic agent. In some embodiments, the hydrogel formulation comprises a non-ionic osmotic agent in the amount of between about 0.5% and about 7.5% (w/w). In some embodiments, the non-ionic osmotic agent is selected from polyethylene glycol (PEG), sorbitol, lactose, fructose, dextrose, lactulose, sucrose, xylitol, mannitol, and mixtures thereof. In some embodiments, the quaternary ammonium silane hydrogel formulation comprises about 5.0% (w/w) of the non-ionic osmotic agent. In some embodiments, the quaternary ammonium silane hydrogel formulation comprises about 5.0% (w/w) of PEG. In some embodiments, the quaternary ammonium silane hydrogel formulation comprises about 5.0% (w/w) of sorbitol. In some embodiments, the quaternary ammonium silane hydrogel formulation comprises between about 1.0% and about 3.0% (w/w) of the non-ionic osmotic agent. In some embodiments, the quaternary ammonium silane hydrogel formulation comprises between about 1.0% and about 3.0% (w/w) PEG. In some embodiments, the quaternary ammonium silane hydrogel formulation comprises between about 1.0% and about 3.0% (w/w) sorbitol. In some embodiments, the quaternary ammonium silane hydrogel formulation comprises about 1.5% and about 2.5% (w/w) of the non-ionic osmotic agent. In some embodiments, the quaternary ammonium silane hydrogel formulation comprises about 1.5% and about 2.5% (w/w) PEG. In some embodiments, the quaternary ammonium silane hydrogel formulation comprises about 1.5% and about 2.5% (w/w) sorbitol. In some embodiments, the quaternary ammonium silane hydrogel formulation comprises about 2.0% (w/w) of the non-ionic osmotic agent. In some embodiments, the quaternary ammonium silane hydrogel formulation comprises about 2.0% (w/w) PEG. In some embodiments, the quaternary ammonium silane hydrogel formulation comprises about 2.0% (w/w) sorbitol. In some embodiments, the non-ionic osmotic agent is PEG with an average molecular weight (Mn) of between about 200 and about 100,000. In some embodiments, the non-ionic osmotic agent is PEG with an average molecular weight (Mn) of about 6000.

As provided herein, the hydrogel formulation comprises an aqueous solution. In some embodiments, the hydrogel formulation comprises an aqueous solution in the amount of between about 75% and about 99.125% (w/w). Suitable aqueous solutions include, for example, but are not limited to, water, saline, or a phosphate buffered saline.

In some embodiments, the aqueous hydrogel formulation comprises:
a) between about 0.25% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.5% and about 2.0% (w/w) of a non-ionic gelling agent;
c) between about 0.05% to and about 0.2% (w/w) of a non-ionic adhesion agent;
d) between about 1.0% and about 3.0% (w/w) of a non-ionic wetting agent;
e) optionally between about 2.0% and about 5.0% (w/w) of a non-ionic osmotic agent; and,
f) optionally one or more additional agents.

In some embodiments of the immediately preceding hydrogel, the non-ionic gelling agent lacks a carboxylic acid group. In some embodiments, the non-ionic wetting agent lacks a carboxylic acid group. In some embodiments, both the non-ionic gelling agent and the non-ionic wetting agent lack a carboxylic acid group. In some embodiments, the hydrogel further comprises a buffer. In some embodiments, the hydrogel further comprises a pH adjusting agent, for example, but not limited to methanesulfonic acid. In some embodiments, the aqueous hydrogel formulation has a pH between about 5 and about 8. In some embodiments, the aqueous hydrogel formulation has a pH between about 5.5 and about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the complete hydrolysis of a compound in the hydrogel selected from the group consisting of Formula A-P and Compounds I-LIV, or a combination thereof;
b) about 2.0% (w/w) of a non-ionic gelling agent that lacks a carboxylic acid group;
c) between about 0.1% and about 0.2% (w/w) of a non-ionic adhesion agent;
d) about 2.0% (w/w) of a non-ionic wetting agent that lacks a carboxylic acid group;
e) about 2.0% (w/w) of a non-ionic osmotic agent; and,
f) optionally one or more additional agents as described herein.
   In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 2.0% (w/w) of HPMC;
c) about 0.1% (w/w) of poloxamer 407;
d) about 2.0% (w/w) of propylene glycol;
e) about 2.0% (w/w) of PEG; and,
f) optionally one or more additional agents as described herein.
   In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 1.0% (w/w) of HPMC;
c) about 0.1% (w/w) of poloxamer 407;
d) about 2.0% (w/w) of propylene glycol;
e) about 2.0% (w/w) of PEG; and,
f) optionally one or more additional agents as described herein.

In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 2.0% (w/w) of a non-ionic gelling agent that lacks a carboxylic acid group;
c) between about 0.1% and about 0.2% (w/w) of a non-ionic adhesion agent;
d) about 2.0% (w/w) of a non-ionic wetting agent that lacks a carboxylic acid group;
e) about 5.0% (w/w) of a non-ionic osmotic agent; and,
f) optionally one or more additional agents as described herein.

In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 2.0% (w/w) of HPMC;
c) about 0.2% (w/w) of poloxamer 407;
d) about 2.0% (w/w) of glycerin;
e) about 5.0% (w/w) of a sorbitol; and,
f) optionally one or more additional agents as described herein.

In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is

Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 1.0% (w/w) of HPMC;
c) about 0.2% (w/w) of poloxamer 407;
d) about 2.0% (w/w) of glycerin;
e) about 5.0% (w/w) of a sorbitol; and,
f) optionally one or more additional agents as described herein.

In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 2.0% (w/w) of a non-ionic gelling agent;
c) about 0.1% (w/w) of a non-ionic adhesion agent;
d) about 2.0% (w/w) of a non-ionic wetting agent; and,
e) optionally one or more additional agents as described herein.

In some embodiments of the immediately preceding hydrogel, the non-ionic gelling agent lacks a carboxylic acid group. In some embodiments, the non-ionic wetting agent lacks a carboxylic acid group. In some embodiments, both the non-ionic gelling agent and the non-ionic wetting agent lack a carboxylic acid group. In some embodiments, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation comprises:
a) about 0.5% (w/w) of a trihydroxy-QAS cation of Formula X that is derived from the hydrolysis of a compound in the hydrogel selected from the group consisting of Formula A-P and Compounds I-LIV, or a combination thereof;
b) about 2.0% (w/w) of HPMC;
c) about 0.2% (w/w) of poloxamer 407;
d) about 2.0% (w/w) of glycerin; and,
e) optionally one or more additional agents as described herein.

In some embodiments of the immediately preceding hydrogel, the hydrogel further comprises a buffer. In some embodiments, the hydrogel further comprises a pH adjusting agent, for example, but not limited to methanesulfonic acid. In some embodiments, the aqueous hydrogel formulation has a pH between about 5.5 and about 6.5. In some embodiments, the pH is less than about 6.5. In some embodiments, the aqueous hydrogel formulation has a pH of about 6. In some embodiments, the aqueous hydrogel formulation has a viscosity of between about 1000 and 2000 cP. In some embodiment, the aqueous hydrogel formulation has a viscosity of between about 1200 and 1800 cP. In some embodiments, the aqueous hydrogel formulation has a viscosity of between about 1250-1600 cP. In some embodiments, the compound used to produce the hydrogel is Compound VII. In some embodiments, the compound used to produce the hydrogel is Compound XIII. In some embodiments, the compound used to produce the hydrogel is Compound XIX. In some embodiments, the trihydroxy-QAS cation is Ion I.

In some embodiments, the aqueous hydrogel formulation optionally further comprises an agent that disrupts and/or destabilizes the extracellular polymeric substances (EPS) that make up a biofilm (an “EPS disrupter”), for example, as described further below.

In some embodiments, the aqueous hydrogel formulation optionally further comprises a demulcent agent, for example, as described further below.

In some embodiments, the aqueous hydrogel formulation optionally further comprises an additional antiseptic or antimicrobial agent, for example, as described further below.

In some embodiments, the aqueous hydrogel formulation optionally further comprises a plaque controlling agent, anti-carries agent, or calculus formation inhibitor, for example, as described further below.

In some embodiments, the aqueous hydrogel formulation optionally further comprises a malodour reducing agent, for example, as described further below.

In some embodiments, the aqueous hydrogel formulation optionally further comprises a chelating agent, for example, as described further below.

In some embodiments, the aqueous hydrogel formulation optionally further comprises a preservative, for example, as described further below.

In some embodiments, the aqueous hydrogel formulation optionally further comprises a buffer, for example, as described further below.

In some embodiments, the aqueous hydrogel formulation optionally further comprises a pH adjusting agent, for example, as described further below.

In one aspect, a method of using a quaternary ammonium silane hydrogel aqueous formulation described herein is provided, comprising applying an effective amount of the aqueous hydrogel formulation to a substrate to inhibit, reduce, or prevent the growth of at least one type of microbe (e.g., in a microbial biofilm). In some embodiments, the aqueous hydrogel formulation is applied to a human or animal to inhibit, reduce, or prevent the growth of at least one type of microbe (e.g., in a microbial biofilm).

In some embodiments, an aqueous hydrogel formulation described herein is applied directly to human or animal tissue, including an oral cavity, e.g., as a topical formulation.

In some embodiments, an aqueous hydrogel formulation described herein is applied directly to a human or animal wound.

In another embodiment, an aqueous hydrogel formulation described herein is present in an article, for example, a dressing, bandage, surgical packing, gauze, wrap, conformable foam, or other suitable device for use in medical applications, for example in a wound dressing or in surgical packings to reduce or prevent the risk of microbial infection.

In another embodiment, an aqueous hydrogel formulation described herein is used in the oral cavity of a human or animal. In a particular embodiment, a method is disclosed for treating or preventing an infection of the tongue (thrush), gum (gingivitis), supragingival plaque, periodontitis, dental abscess, stomatitis, pharyngitis, facial cellulitis, tooth decay, halitosis, or plaque in a subject in need thereof, comprising administering an effective amount of an aqueous hydrogel formulation described herein to the subject, thereby treating the infection.

In another embodiment, an aqueous hydrogel formulation described herein is used in treating a microbial infection in or on a human fingernail or toenail.

In a fourth aspect, a kit is provided comprising a vial comprising a quaternary ammonium silane hydrogel formulation as described herein and an application device. In some embodiments, the application device is a syringe. In some embodiments, the application device is a balloon-tipped catheter.

### BRIEF DESCRIPTION OF THE DRAWINGS

FIG. 1: Depicts a general scheme of the hydrolysis of a compound of the invention: reaction depicted under a) corresponds to the hydrolysis of the compound where the positive charge of trihydroxy quaternary ammonium silyl cation is compensated by an external anion; reaction b) corresponds to the hydrolysis of the compound where the positive charge of trihydroxy quaternary ammonium silyl cation is compensated by an internal anion present in a side chain group of the compound containing ionizable acidic group-XH.

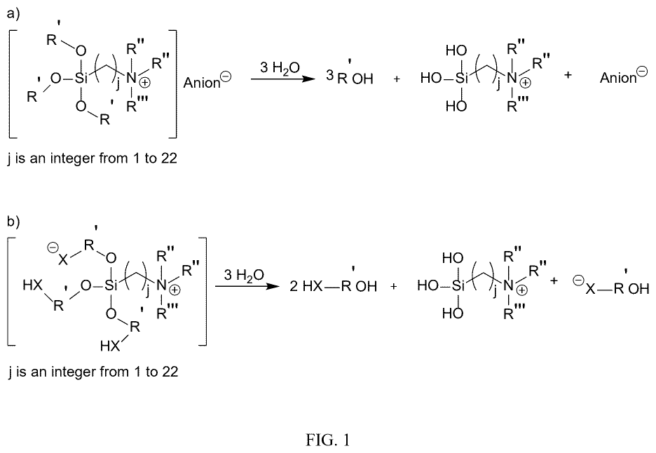

FIG. 2 is a mass-spectrum of Compound VII (N,N-dimethyl-N-(3-(tris(2-hydroxypropoxy) silyl)propyl)octadecan-1-aminium methane sulfonate) in propylene glycol diluted with acetonitrile. The mass-spectrum contains two major peaks: a peak with m/z of 418.4 corresponding to N,N-dimethyl-N-(3-(trihydroxysilyl)propyl)octadecan-1-aminium cation formed as a result of complete hydrolysis of Compound VII and a peak with m/z of 476.5 corresponding to N-(3-(dihydroxy (2-hydroxypropoxy) silyl)propyl)-N,N-dimethyloctadecan-1-aminium cation formed as a result of partial hydrolysis of Compound VII, where only two side chains of Compound VII were hydrolyzed.

FIG. 3 is a mass-spectrum of Compound VII diluted in water and recorded at a 30-minute time point. The mass-spectrum contains one major peak with m/z of 418.4 corresponding to N,N-dimethyl-N-(3-(trihydroxysilyl)propyl)octadecan-1-aminium cation formed as a result of complete hydrolysis of Compound VII.

FIG. 4 is an extracted ion chromatogram of the cation with m/z of 418.

FIG. 5 is a mass-spectrum of Compound VII formulated with inactive ingredients and diluted in water. The mass-spectrum contains one major peak with m/z of 418.4 corresponding to N, N-dimethyl-N-(3-(trihydroxysilyl)propyl)octadecan-1-aminium cation formed as a result of complete hydrolysis of Compound VII.

FIG. 6: Next generation sequencing data of oral microbiome in canine pre-treatment with quaternary ammonium silane hydrogel. Data represents the trimmed taxa of bacterial genera present in the upper and lower dental arcade of a canine patient. The trimmed taxa files are generated using the full taxa data after the confidence values have been taken into account. As such each taxonomic level is assigned only if the confidence value is greater than or equal to .51 (51%). If a taxon falls below 0.51, it is replaced with the “Unknown” keyword. Evaluation was performed using Next Generation Sequencing in partnership with RTL Genomics, LLC.

FIG. 7: Next generation sequencing data of oral microbiome in canine 212 Days post-treatment with quaternary ammonium silane hydrogel. Taxa represents the trimmed data of bacterial genera present in the upper and lower dental arcade of a canine patient. Evaluation was performed using Next Generation Sequencing in partnership with RTL Genomics, LLC.

FIG. 8: Single treatment of quaternary ammonium silane hydrogel causes decrease of Fusobacterium species in canine gingiva. Violin plot showing relative abundance of bacterial genus present in the oral cavity. Solid red lines indicate median; dashed red lines indicates 1^{st} and 4^{th} quartiles. Each point represents an individual animal.

FIG. 9: Single treatment of quaternary ammonium silane hydrogel causes decrease of Porphyromonas species in canine gingiva. Violin plot showing relative abundance of bacterial genus present in the oral cavity. Solid red lines indicate median; dashed red lines indicates 1^{st} and 4^{th} quartiles. Each point represents an individual animal.

FIG. 10: Single treatment of quaternary ammonium silane hydrogel causes decrease of Moraxella species in canine gingiva. Violin plot showing relative abundance of bacterial genus present in the oral cavity. Solid red lines indicate median; dashed red lines indicates 1^{st} and 4th quartiles. Each point represents an individual animal.

FIG. 11: Single treatment of quaternary ammonium silane hydrogel causes decrease of Neisseria species in canine gingiva. Violin plot showing relative abundance of bacterial genus present in the oral cavity. Solid red lines indicate median; dashed red lines indicates 1^{st} and 4th quartiles. Each point represents an individual animal.

FIG. 12: Single treatment of quaternary ammonium silane hydrogel causes decrease of Clostridiales fm. species in canine gingiva. Violin plot showing relative abundance of bacterial genus present in the oral cavity. Solid red lines indicate median; dashed red lines indicates 1^{st} and 4^{th} quartiles. Each point represents an individual animal.

FIG. 13: Single treatment of quaternary ammonium silane hydrogel causes decrease of unclassified bacterial species in canine gingiva. Violin plot showing relative abundance of bacterial genus present in the oral cavity. Solid red lines indicate median; dashed red lines indicates 1^{st} and 4^{th} quartiles. Each point represents an individual animal.

### DETAILED DESCRIPTION OF THE INVENTION

Provided herein are advantageous aqueous hydrogel formulations comprising a compound selected from the group consisting of Formula A-P and Compounds I-LIV, methods of using said formulations, and methods of reducing, eliminating, or preventing polymicrobial infections with the described formulations.

### Definitions

Compounds are described using standard nomenclature. Unless defined otherwise, all technical and scientific terms used herein have the same meaning as is commonly understood by one of skill in the art to which this invention belongs.

The quaternary ammonium silanes in any of the Formulas described herein include racemates, enantiomers, mixtures of enantiomers, diastereomers, mixtures of diastereomers, as if each is specifically described.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “for example” or “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

As used herein, the term “about” means “+/−10%”.

In some embodiments, the quaternary ammonium silane of the aqueous hydrogel formulation includes at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into quaternary ammonium silanes of the invention include isotopes of hydrogen, carbon, nitrogen, oxygen, and silicon, such as ²H, ³H, ¹¹C, ¹³C, ¹⁴C, ¹⁵N, ¹⁷O, ¹⁸O, ²⁹Si, and ³⁰Si, respectively.

By way of general example and without limitation, isotopes of hydrogen, for example, deuterium (²H) and tritium (³H) may be used anywhere in described structures that achieves the desired result. Alternatively, or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least about 90, 95 or 99% or more enriched in an isotope at any location of interest. In one non-limiting embodiment, deuterium is at least about 90, 95 or 99% enriched at a desired location.

In one non-limiting embodiment, the substitution of one or more hydrogen atoms for a deuterium atom can be provided in any of the compounds described herein. In one non-limiting embodiment, the substitution of a hydrogen atom for a deuterium atom occurs within a group described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.).

In certain embodiments a quaternary ammonium silane described herein may form a solvate with one or more solvents (for example water). Therefore, in one non-limiting embodiment, the invention includes a solvated form of the quaternary ammonium silane. The term “solvate” refers to a molecular complex of a compound described herein (including a salt thereof) with one or more solvent molecules. Non-limiting examples of solvents include water. The term “hydrate” refers to a molecular complex comprising an quaternary ammonium silane of the invention and water. Pharmaceutically acceptable solvates in accordance with the invention include those wherein the solvent may be isotopically substituted, e.g., D₂O. A solvate can be in a liquid or solid form.

A dash (“-”) that is not between two letters or symbols is used to indicate a point of attachment for a substituent. For example, —(C═O)NH₂ is attached through carbon of the keto (C═O) group.

The term “carrier” applied to compositions/combinations of the invention refers to a diluent, excipient, or vehicle with which an active compound is provided.

A “patient” or “host” or “subject” is a human or non-human animal in need of treatment or prevention of any of an infection as described herein. In some embodiments, the host is a human. A “patient” or “host” or “subject” also refers to for example, a mammal, primate (e.g., human), cows, sheep, goat, horse, dog, cat, rabbit, rat, mice, fish, bird and the like.

“Halo” and “Halogen” is fluorine, chlorine, bromine, or iodine.

A “therapeutically effective amount” of a composition/combination of this invention means an amount effective, when administered to a host, to provide a therapeutic benefit such as an amelioration of symptoms or reduction or diminution of the disease itself. In some embodiments, a therapeutically effective amount is an amount sufficient to inhibit progression, cause a regression, cause a cure, or inhibit, eliminate, or prevent an infection in a host in need thereof.

As used herein, the term “interval” refers to a period of time after administration of the formulation disclosed herein to a substrate (e.g., a tissue). The interval can be any suitable period of time, for example, 1, 5, 10, 25, 30, 45, 50 or 60 minutes. In certain embodiments, the interval is about 1 day, 2 days, 3 days, 4 days, 5 day or a week or more.

As used herein, “salt” is a derivative of the disclosed compound in which the parent compound is modified by making inorganic or organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reactive free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practical. Salts of the present compounds further include solvates of the compound and the compound salts.

Examples of salts include, but are not limited to, mineral or organic acid salts of basic residues such as amines; alkali or organic salts of acidic residues such as carboxylic acids; and the like. The salts include the conventional non-toxic salts and the ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)n-COOH where n is 0-4, and the like, or using a different acid that produces the same counterion. Lists of additional suitable salts may be found, e.g., in Remington's Pharmaceutical Sciences, 17th 20 ed., Mack Publishing Company, Easton, Pa., p. 1418 (1985).

As used herein, “% weight/weight” or “% w/w” is the expression of the weight concentration of a solution. The weight of each constituent of the formulation is used. By way of non-limiting example, if the total mass of a 100-gram solution is made up of 0.5 g of quaternary ammonium silane in 99.5 g of water then the quaternary ammonium silane concentration is expressed as 0.5% weight/weight.

### Quaternary Ammonium Silanes of the Ammonium Silane Hydrogel Formulation

The aqueous hydrogel formulations provided herein comprise a quaternary ammonium silane compound selected from the group consisting of Formula A-P and Compounds I-LIV, or a combination thereof. Importantly, the quaternary ammonium silane compounds for use in the aqueous hydrogel formulation provided herein are completely hydrolyzed during the formation of the hydrogel resulting in i) a cationic trihydroxy quaternary ammonium silane moiety (“trihydroxy-QAS cation”); ii) a anionic counterion; and iii) a smaller hydroxyl-containing product derived upon the hydrolysis from the side chains attached to the oxygens of the Si—O moiety of the compound. The trihydroxy-QAS cation produced by the hydrolysis of the compounds described herein is of Formula X:

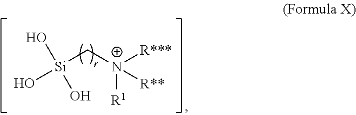

wherein
   r is 1, 2, 3, or 4;
   R¹ is C₆-C₂₂alkyl or C₆-C₂₂ alkanoyl;
   R** and R*** are independently selected from the group consisting of:
   i. C₁-C₄ alkyl; and
   ii. C₆-C₂₂alkyl or C₂-C₂₂alkanoyl.

In some embodiments, the trihydroxy-QAS cation produced is Ion I:

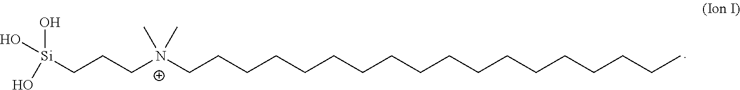 [SMILES: CCCCCCCCCCCCCCCCCC[N+](C)(C)CCC[Si](O)(O)O]

In certain embodiments a quaternary ammonium silane compound for use in the aqueous hydrogel formulation is selected from the group consisting of compound of Formula A and compound of Formula B:

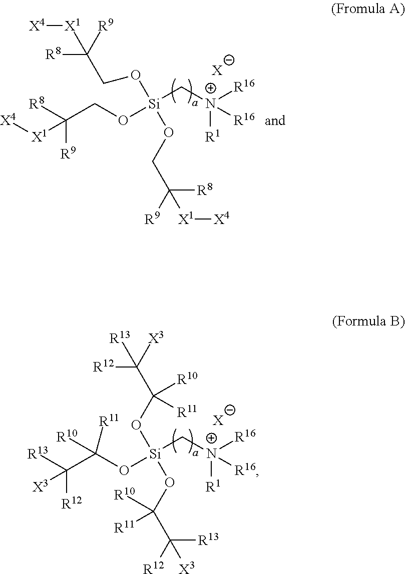

wherein:
a is 1, 2, 3, or 4;
R¹ is independently at each occurrence selected from the group consisting of C₆-C₂₂alkyl
and C₆-C₂₂ alkanoyl;
R⁸ is H or CH₂OH;
R⁹ is H or C₁-C₈ alkyl;
X³ is OH or CH₂OR⁷;
R¹⁰, R¹¹, and R¹² are independently at each occurrence selected from the group consisting of hydrogen, hydroxyl, CH₂OR⁷, CON(R⁷)₂, COOR⁷, C(O)R⁷, C₁-C₈ alkyl, C₁-C₈ hydroxyalkyl, and C₁-C₈ alkanoyl;
R¹³ is selected from the group consisting of hydroxyl, CH₂OR⁷, CON(R⁷)₂, COOR⁷, C(O)R⁷, C₁-C₈ alkyl, C₁-C₈ hydroxyalkyl, and C₁-C₈ alkanoyl;
R⁷ is independently at each occurrence selected from hydrogen, C₁-C₈ alkyl, C₁-C₈ hydroxyalkyl, and C₁-C₈ alkanoyl;
X¹ is NR¹⁷, CH₂, or C(O);
X⁴ is selected from the group consisting of hydroxyl, CH₂OR⁷, CON(R⁷)₂, COOR⁷, C(O)R⁷, C₁-C₈ hydroxyalkyl, and C₁-C₈ alkanoyl;
R¹⁶ is C₁-C₄ alkyl;
R¹⁷ is hydrogen, C₁-C₈ hydroxyalkyl, or C₁-C₈ alkanoyl;
X⁻ is an anion.

In certain embodiments the anion X⁻ in a compound of the invention is selected from the group consisting of chloride, fluoride, iodide, bromide, hydroxide, chlorite, chlorate, hydroxide, formate, acetate, lactate, benzoate, methane sulfonate (mesylate MeSO₃⁻), and salicylate anion.

In certain embodiments a quaternary ammonium silane compound for use in the aqueous hydrogel formulation is selected from the group consisting of compound of Formula A-1 and compound of Formula B-1:

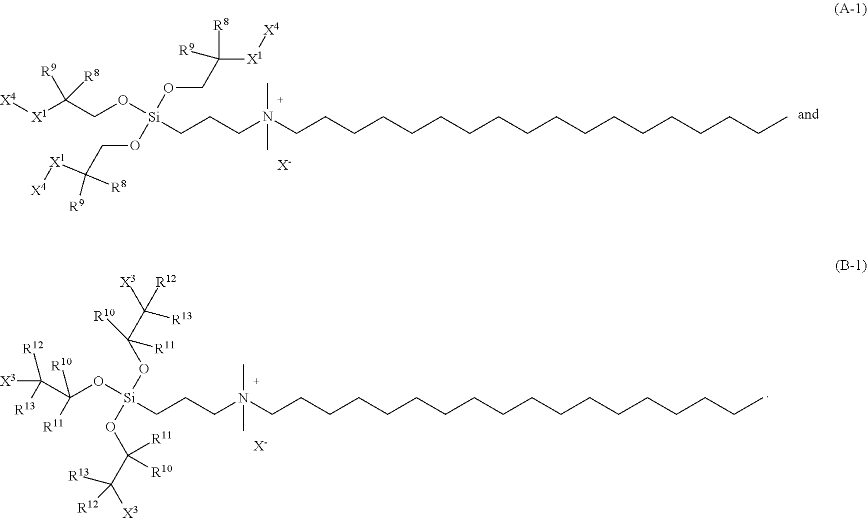

In certain embodiments a quaternary ammonium silane compound for use in the aqueous hydrogel formulation is selected from the group consisting of compound of Formula A-2 and compound of Formula B-2:

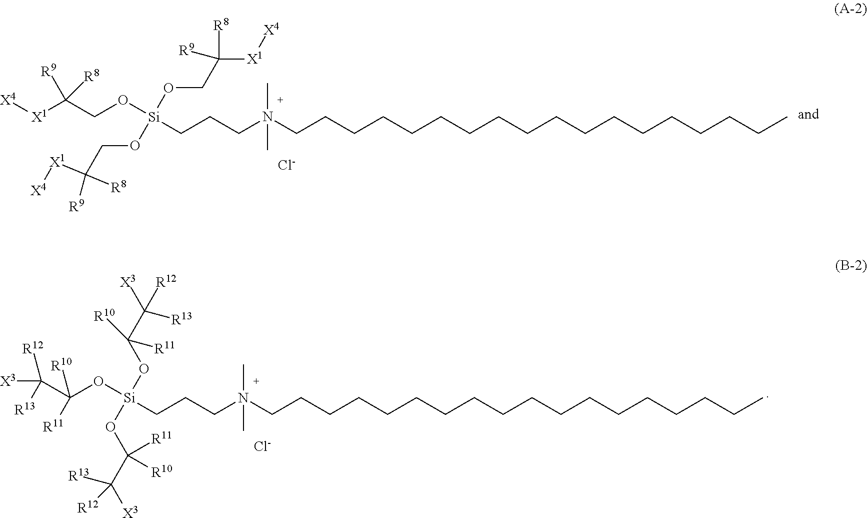

In certain embodiments a quaternary ammonium silane compound for use in the aqueous hydrogel formulation is selected from the group consisting of compound of Formula A-3 and compound of Formula B-3:

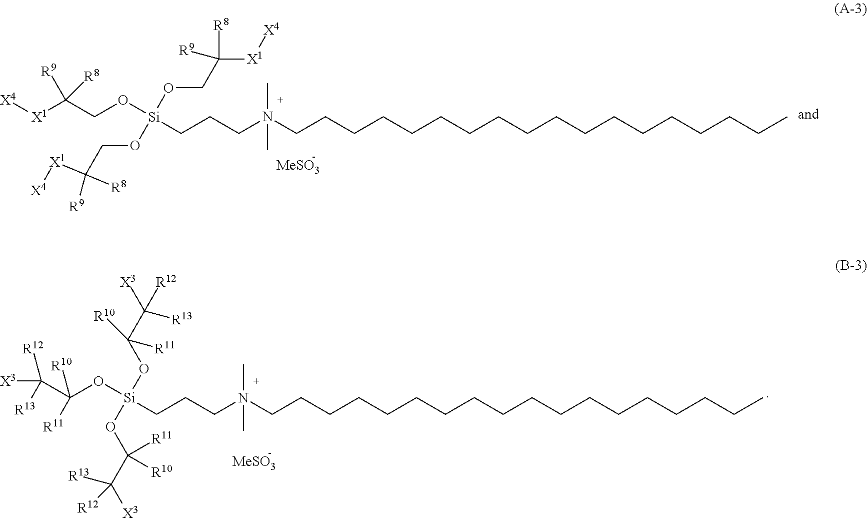

In certain embodiments a quaternary ammonium silane compound for use in the aqueous hydrogel formulation is selected from the group consisting of:

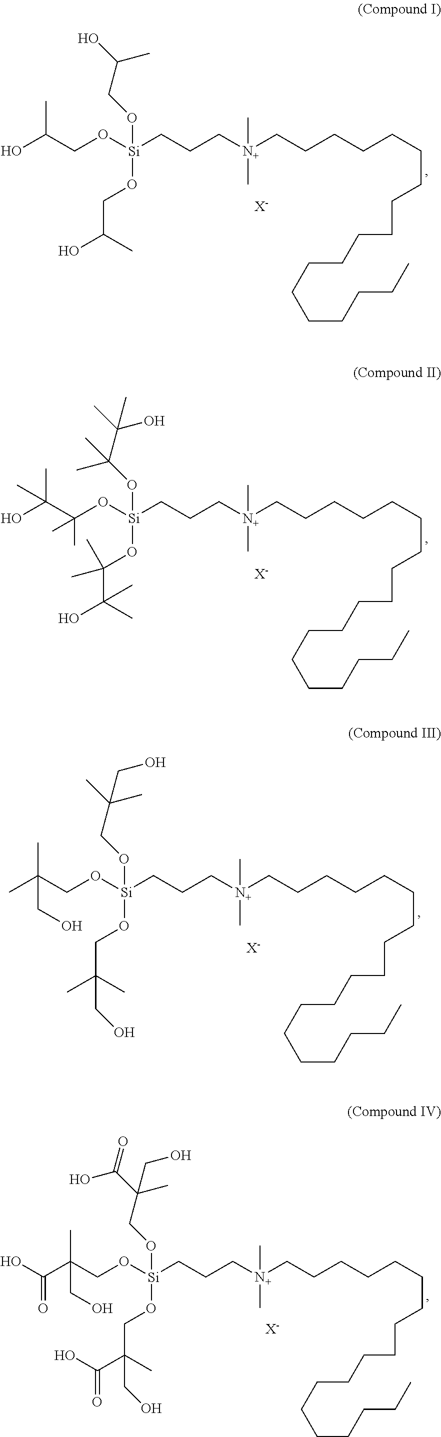

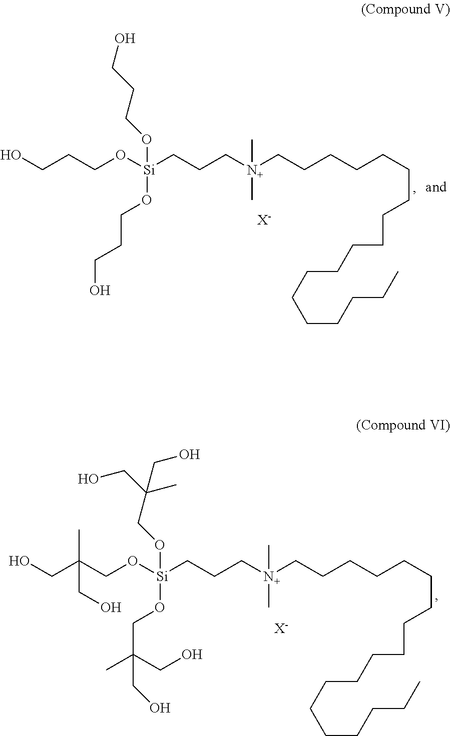

In certain embodiments a quaternary ammonium silane compound for use in the aqueous hydrogel formulation is selected from the group consisting of:

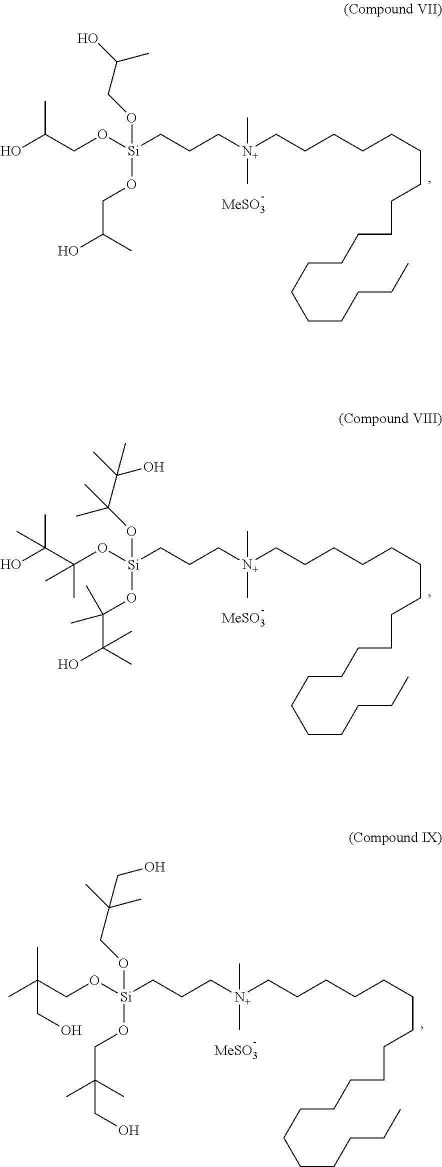 [SMILES: "MeSO3" ; "MeSO3" ; CCCCCCCCCCCCCCCCCC[N+](C)(C)CCC[Si](OCC(C)O)(OCC(C)O)OCC(C)O ; "MeSO3" ; CCCCCCCCCCCCCCCCCC[N+](C)(C)CCC[Si](OC(C)(C)C(C)(C)O)(OC(C)(C)C(C)(C)O)OC(C)(C)C(C)(C)O ; CCCCCCCCCCCCCCCCCC[N+](C)(C)CCC[Si](OCC(C)(C)CO)(OCC(C)(C)CO)OCC(C)(C)CO]

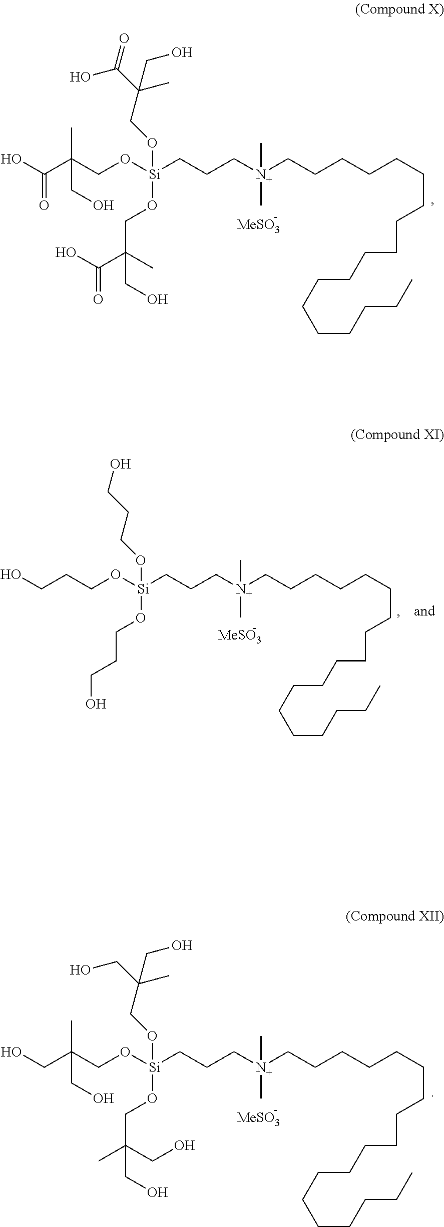 [SMILES: "MeSO3" ; "MeSO3" ; CCCCCCCCCCCCCCCCCC[N+](C)(C)CCC[Si](OCC(C)(CO)C(=O)O)(OCC(C)(CO)C(=O)O)OCC(C)(CO)C(=O)O ; "MeSO3" ; CCCCCCCCCCCCCCCCCC[N+](C)(C)CCC[Si](OCCCO)(OCCCO)OCCCO ; CCCCCCCCCCCCCCCCCC[N+](C)(C)CCC[Si](OCC(C)(CO)CO)(OCC(C)(CO)CO)OCC(C)(CO)CO]

In certain embodiments a quaternary ammonium silane compound for use in the aqueous hydrogel formulation is selected from the group consisting of:

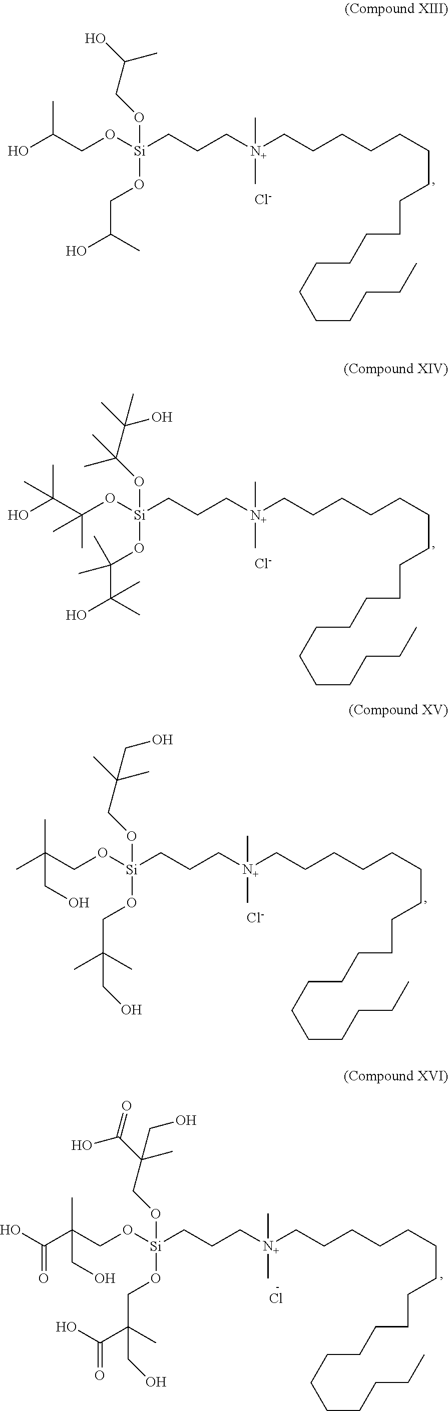

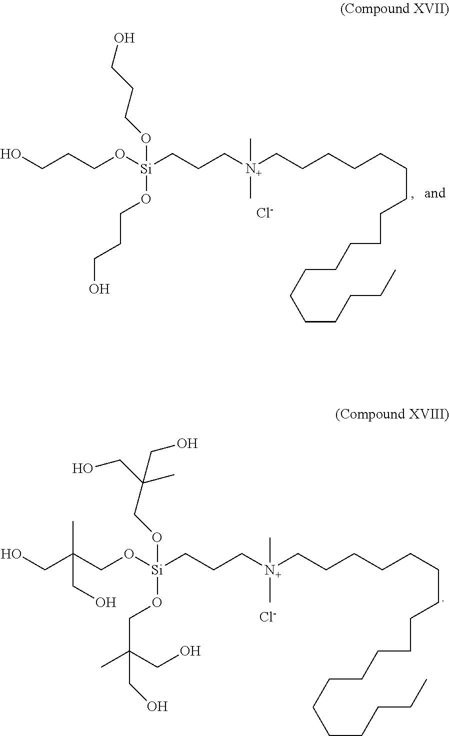 [SMILES: CCCCCCCCCCCCCCCCCC[N+](C)(C)CCC[Si](OCCCO)(OCCCO)OCCCO ; [Cl-] ; CCCCCCCCCCCCCCCCCC[N+](C)(C)CCC[Si](OCC(C)(CO)CO)(OCC(C)(CO)CO)OCC(C)(CO)CO ; [Cl-]]

In certain embodiments a quaternary ammonium silane compound for use in the aqueous hydrogel formulation is selected from the group consisting of compound of Formula C and compound of Formula D:

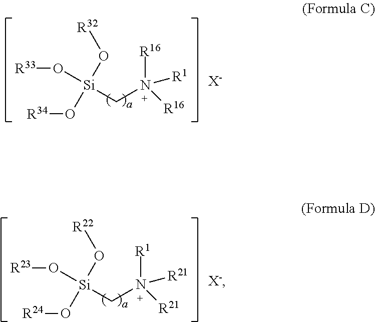

wherein
a is 1, 2, 3, or 4;
R¹ is independently at each occurrence selected from C₆-C₂₂ alkyl and C₆-C₂₂ alkanoyl;
R³², R³³, R³⁴, R²², R²³, and R²⁴ are independently at each occurrence selected from the group consisting of:

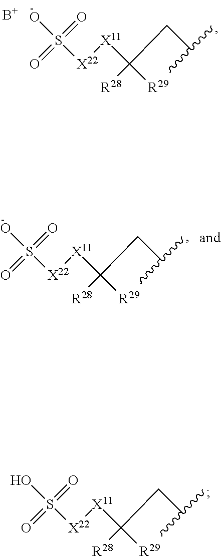

each R²¹ is independently selected from C₁-C₂₂alkyl and C₂-C₂₂ alkanoyl;
R⁷ is independently at each occurrence selected from hydrogen, C₁-C₈ alkyl, C₁-C₈ hydroxyalkyl, and C₁-C₈ alkanoyl;
R²⁸ and R²⁹ are independently at each occurrence selected from hydrogen, halogen, hydroxyl, N (R⁷)₂, CH₂OR⁷, CON(R⁷)₂, COOR⁷, C(O)R⁷, C₁-C₈ alkyl, C₁-C₈ hydroxyalkyl, and C₁-C₈ alkanoyl;
X¹¹ is NR¹⁷, CH₂, CHOH, or C(O);
X²² is C₁-C₃ alkyl or C₁-C₃ hydroxyalkyl;
R¹⁶ is C₁-C₄ alkyl;
R¹⁷ is hydrogen, C₁-C₈ hydroxyalkyl, or C₁-C₈ alkanoyl;
X⁻ is an anion or is absent if the quaternary amine is balanced with an internal anion; and
B⁺ is a cation.

In certain embodiments the cation B⁺ in a compound of the invention is selected from the group consisting of potassium cation, sodium cation, lithium cation, cesium cation, magnesium cation, calcium cation, ammonium cation.

In certain embodiments a quaternary ammonium silane compound for use in the aqueous hydrogel formulation is selected from the group consisting of:

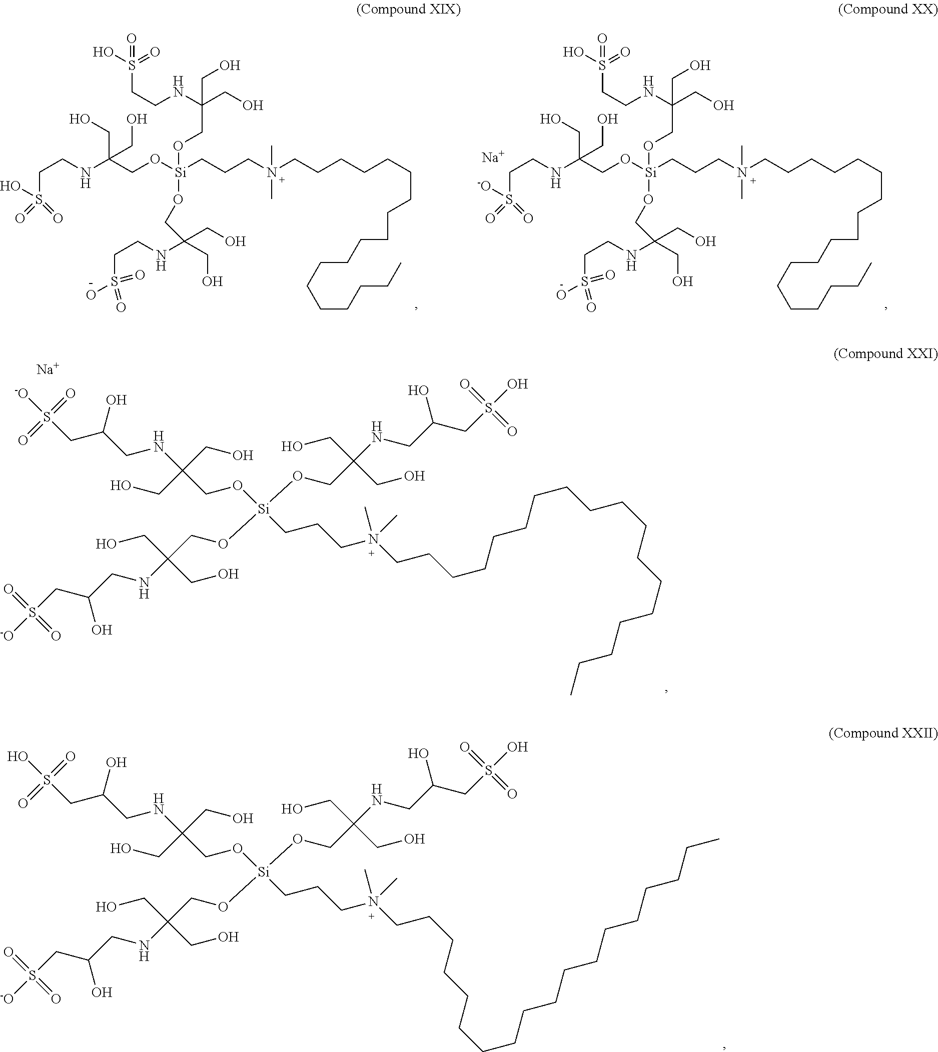

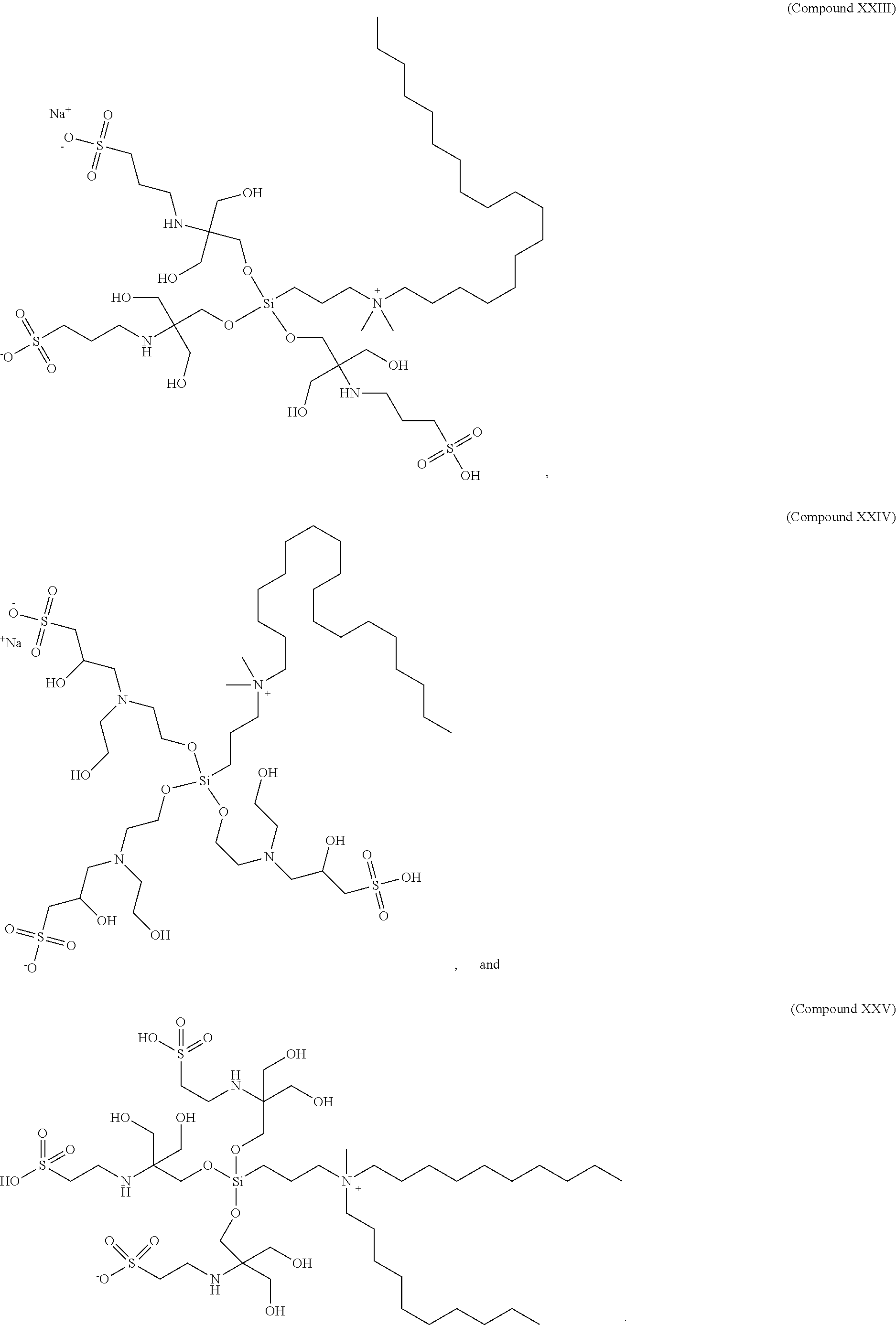

In certain embodiments a quaternary ammonium silane compound for use in the aqueous hydrogel formulation is selected from the group consisting of compound of Formula E and compound of Formula F:

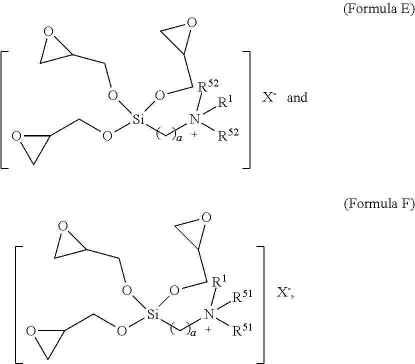

wherein
a is 1, 2, 3, or 4;
R¹ is independently at each occurrence selected from C₆-C₂₂alkyl and C₆-C₂₂ alkanoyl;
R⁵² is independently at each occurrence selected from the group consisting of C₁-C₄alkyl and C₁-C₄ haloalkyl;
each R⁵¹ is independently selected from C₁-C₂₂alkyl and C₂-C₂₂ alkanoyl; each of which R⁵¹ is optionally substituted with 1, 2, or 3, substituents independently selected from C₁-C₆ alkyl and halogen; and
X⁻ is an anion.

In certain embodiments the anion X⁻ in a compound of the invention is selected from the group consisting of chloride, fluoride, iodide, bromide, hydroxide, chlorite, chlorate, hydroxide, formate, acetate, lactate, benzoate, methane sulfonate (mesylate MeSO₃⁻), and salicylate anion.

In certain embodiments a quaternary ammonium silane compound for use in the aqueous hydrogel formulation is selected from the group consisting of

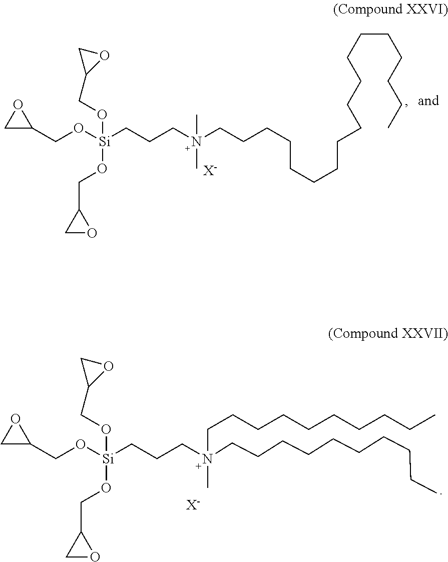

In certain embodiments a quaternary ammonium silane compound for use in the aqueous hydrogel formulation is selected from the group consisting of

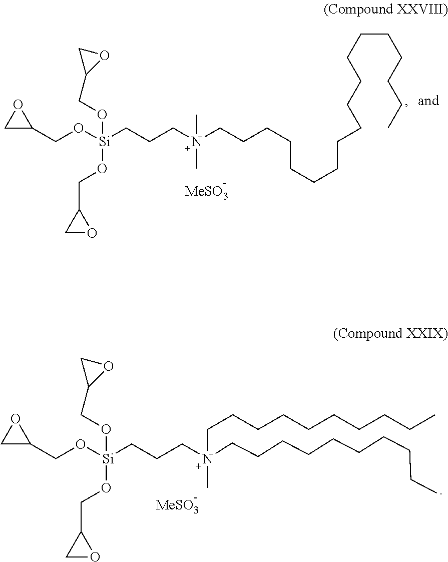

In certain embodiments a quaternary ammonium silane compound for use in the aqueous hydrogel formulation is selected from the group consisting of

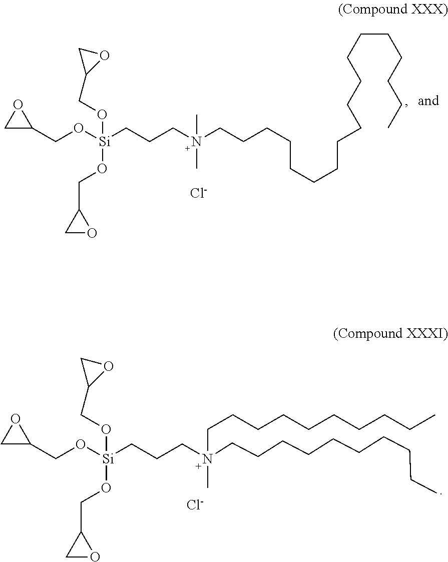

In certain embodiments a quaternary ammonium silane compound for use in the aqueous hydrogel formulation is selected from the group consisting of compound of Formula G, compound of Formula H, compound of Formula I, compound of Formula J, and compound of Formula K:

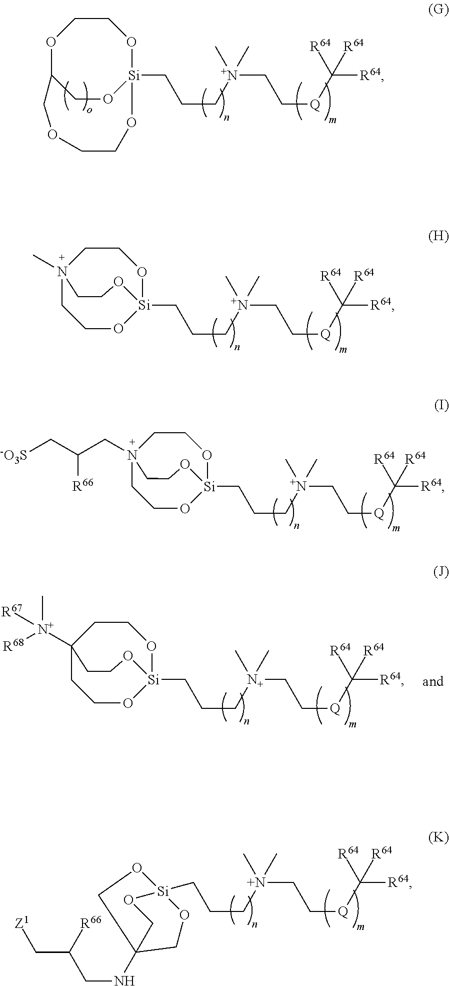

wherein the quaternary amine has a balancing pharmaceutically acceptable anion X⁻;
wherein
m is 5, 6, 7, 8, 9, 10, 11, 12, 13, 14, 15, 16, or 17;
n is 0, 1, or 2;
is 1 or 2;
Q is —CR⁶⁴R⁶⁴—;
R⁶⁴ is independently at each occurrence selected from hydrogen, C₁₋₃ alkyl, C₂₋₄ alkenyl, C₂₋₄alkynyl, halogen, and C₁₋₃ haloalkyl;
R⁶⁶ is independently at each occurrence selected from hydrogen, hydroxy, and C₁-C₆ alkoxy;
R⁶⁷ and R⁶⁸ are independently at each occurrence selected from C₁-C₆ alkyl;
Z¹ is independently at each occurrence selected from

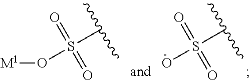

M¹ is hydrogen, sodium, potassium, cesium or lithium;
X⁻ is an anion.

In certain embodiments the anion X⁻ in a compound of the invention is selected from the group consisting of chloride, fluoride, iodide, bromide, hydroxide, chlorite, chlorate, hydroxide, formate, acetate, lactate, benzoate, methane sulfonate (mesylate MeSO₃⁻), and salicylate anion.

In certain embodiments a quaternary ammonium silane compound according to the invention is selected from the group consisting of

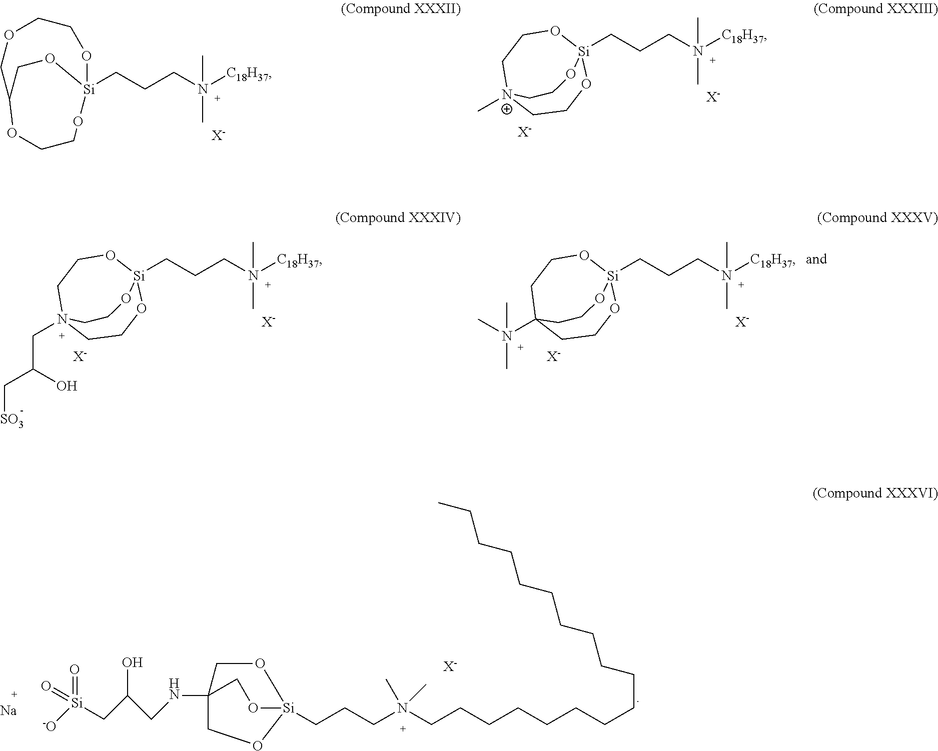

In certain embodiments a quaternary ammonium silane compound for use in the aqueous hydrogel formulation is selected from the group consisting of

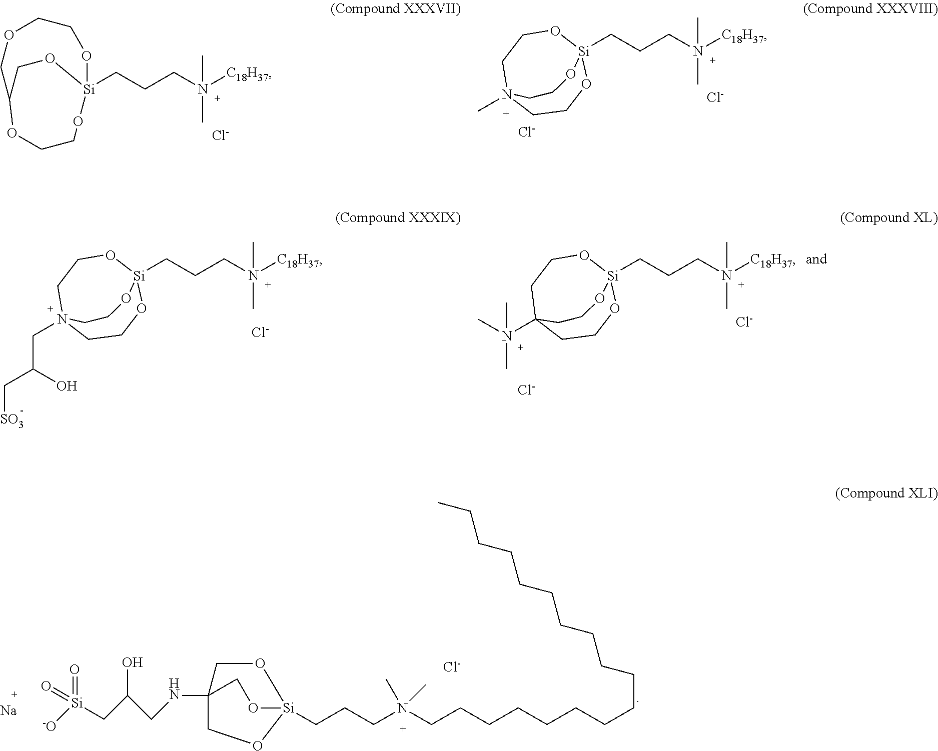

In certain embodiments a quaternary ammonium silane compound for use in the aqueous hydrogel formulation is selected from the group consisting of

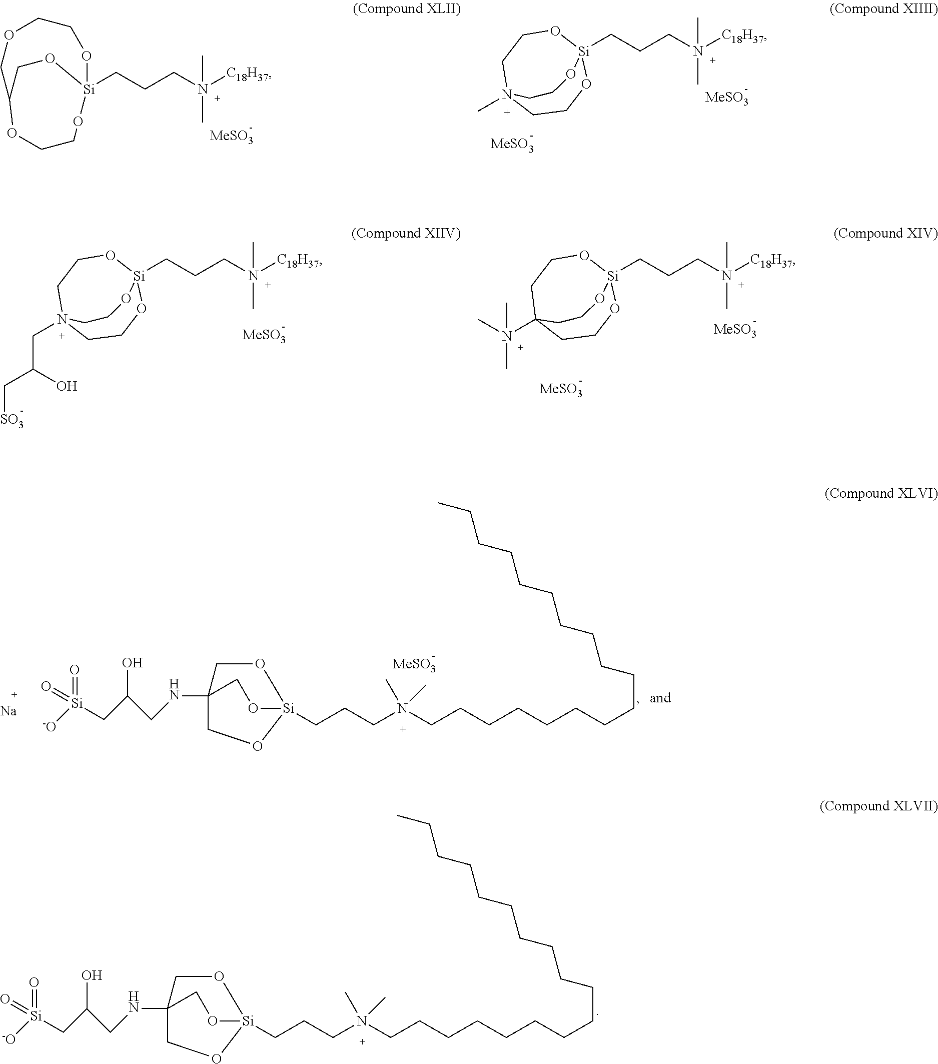

In certain embodiments a quaternary ammonium silane compound for use in the aqueous hydrogel formulation is selected from the group consisting of compound of Formula L, compound of Formula M, and compound of Formula N:

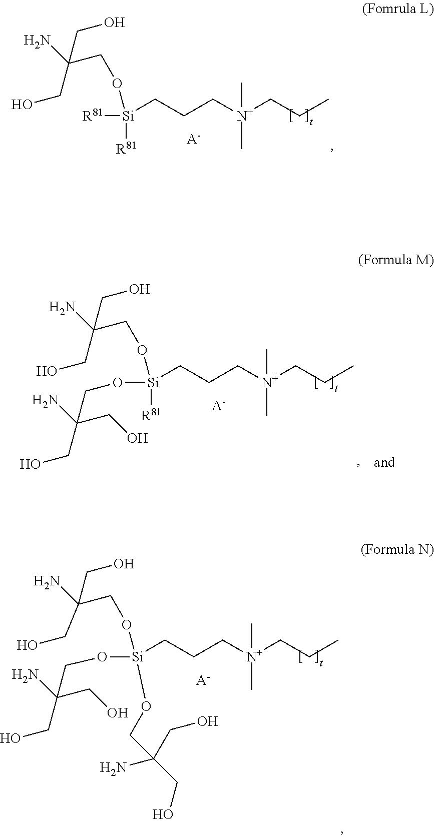

wherein:
each R⁸¹ is independently selected from the group consisting of OH and alkoxy;
A⁻ is a non-halogen anion selected from the group consisting of

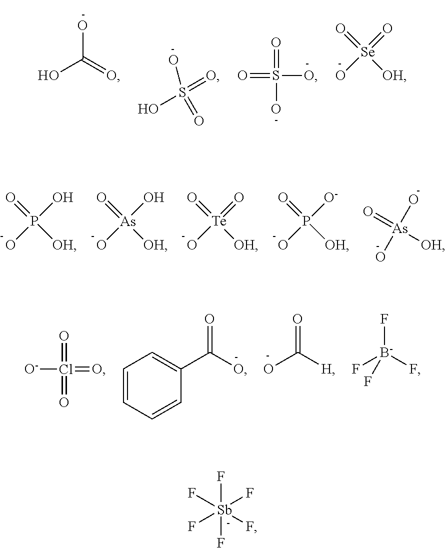

chlorite, perchlorate, hydroxide, formate, acetate, lactate, benzoate, nitrate, and salicylate anion; and
each t is independently selected from the group consisting of 1, 2, 3, 4, 5, 6, 7, 8, 9, 10, 11, 12, 13, 14, 15, 16, 17, and 18.

In certain embodiments a quaternary ammonium silane compound for use in the aqueous hydrogel formulation is selected from the group consisting of

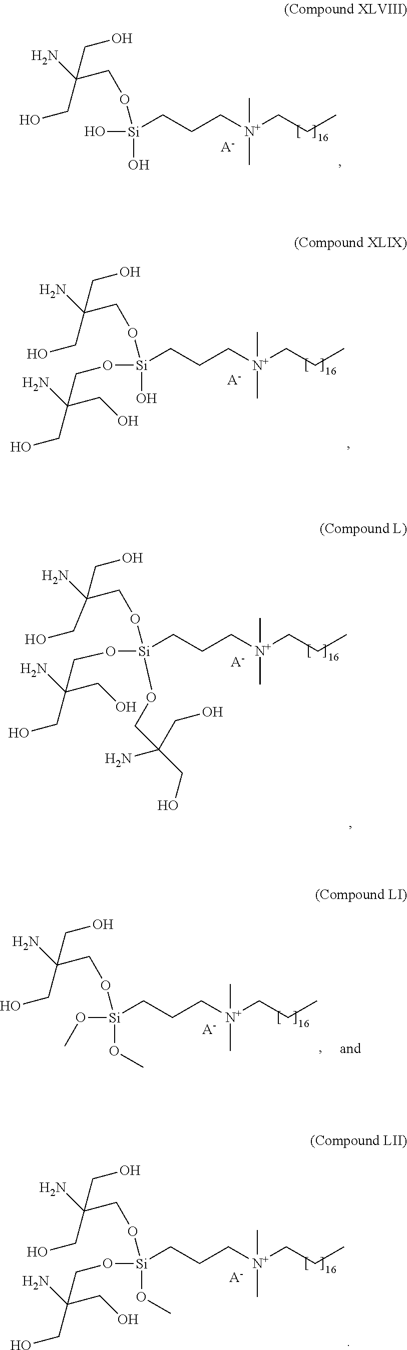

In certain embodiments a quaternary ammonium silane compound for use in the aqueous hydrogel formulation is selected from the group consisting of compound of Formula O and compound of Formula P:

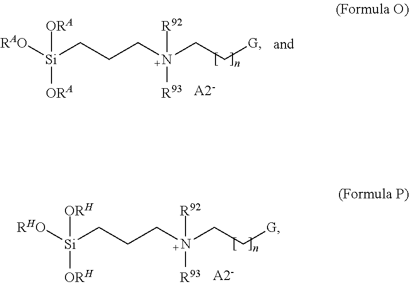

wherein:
R^{A} is selected from:

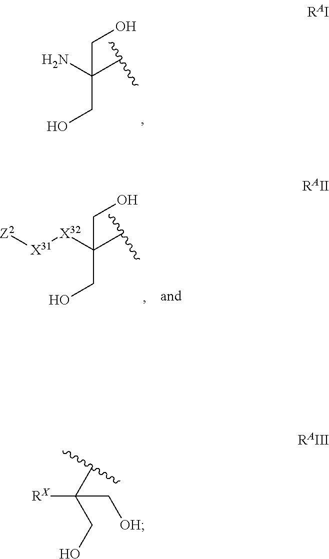

R^{H} is selected from:

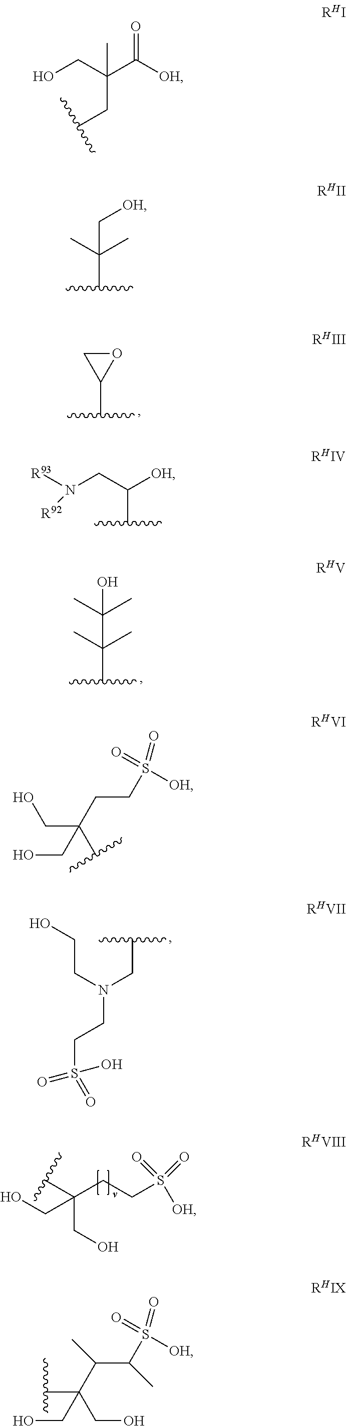

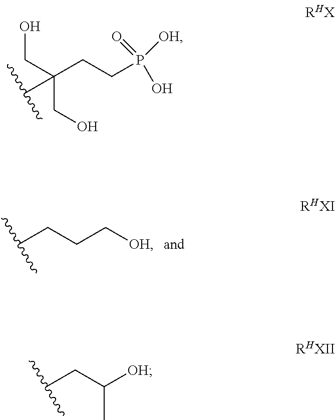

G is hydrogen, alkyl, aryl, cycloalkyl, heterocycle, heteroaryl, —CN, or CF₃;
each R⁹² is selected from hydrogen, C₁-C₆ alkyl, and —O;
R⁹³ is selected from hydrogen, C₁-C₆ alkyl, and —O;
x³¹ is selected from the group consisting of a bond,

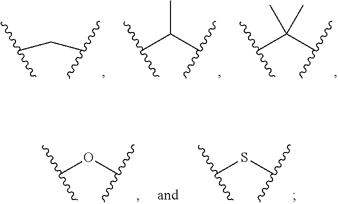

X³² is selected from the group consisting of a bond,

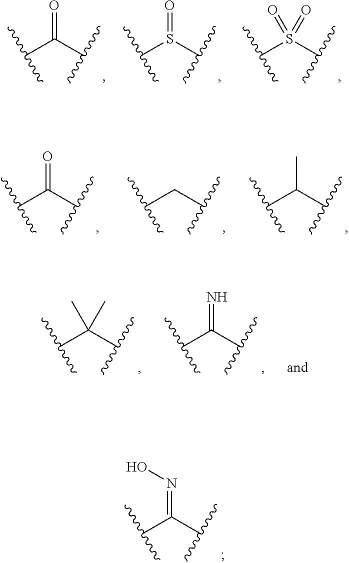

A2⁻ is an anion other than halogen or is absent if the compound of Formula O or Formula P is a charge balanced zwitterion, and wherein A2⁻ is selected from

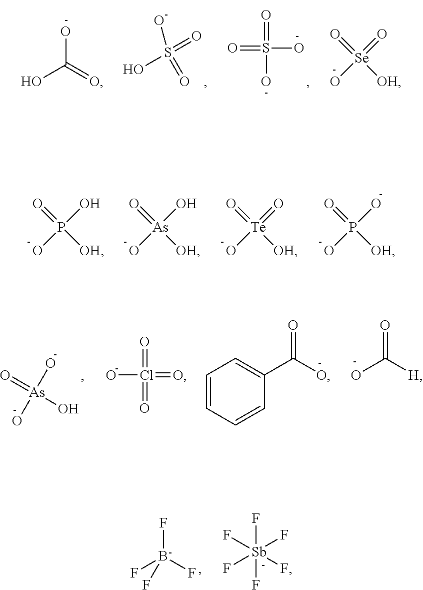

chlorite, perchlorate, hydroxide, formate, acetate, lactate, benzoate, nitrate, and salicylate anion;
R^{X} is selected from

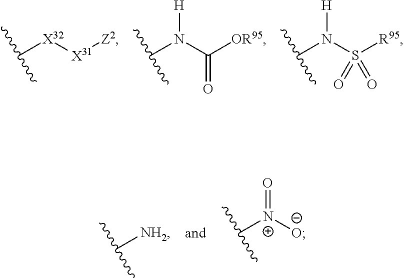

each R⁹⁵ is selected from hydrogen and C₁-C₆ alkyl;
v is selected from the group consisting of 1, 2, 3, 4, 5, 6, 7, 8, 9, 10, 11, 12, 13, 14, 15, 16, 17, and 18;
Z² is selected from alkyl, —NO₂, —SO₃H, —SO₂H, —SO₃, —SO₃M², —CO₂H, —CO₂, —CO₂M², phosphate, OH, NH₂, and SH; and
M² is a metal cation selected from potassium cation, sodium cation, lithium cation, magnesium caution, and calcium cation.

In certain embodiments a quaternary ammonium silane compound for use in the aqueous hydrogel formulation is selected from the group consisting of

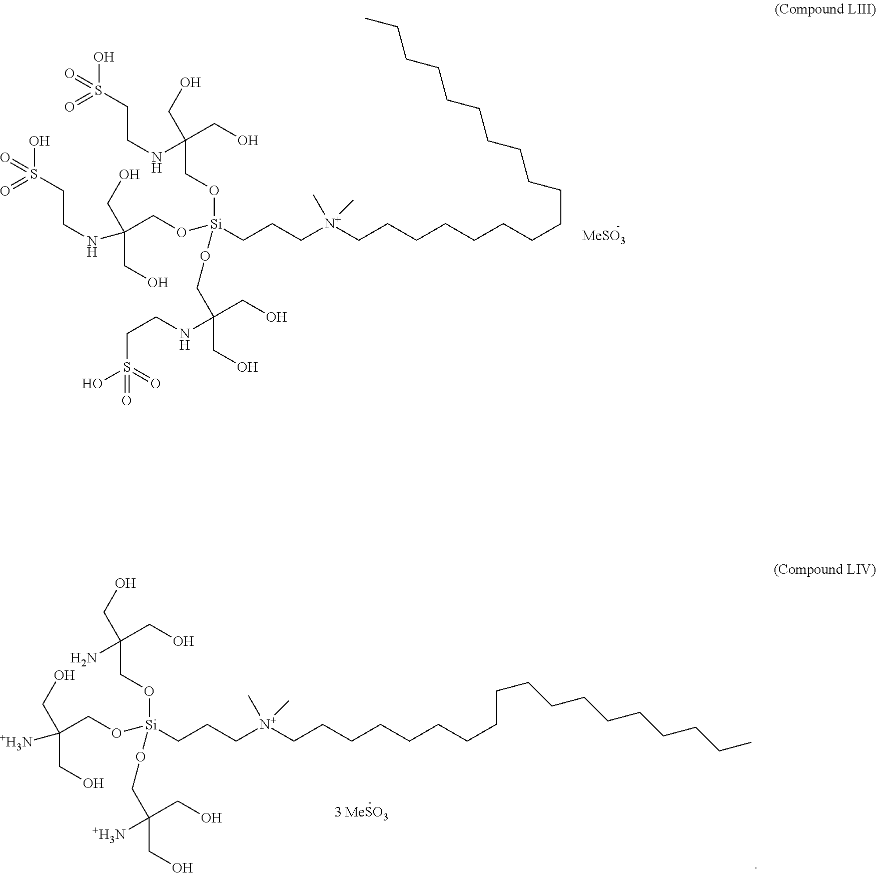 [SMILES: CCCCCCCCCCCCCCCCCC[N+](C)(C)CCC[Si](OCC(CO)(CO)NCCS(=O)(=O)O)(OCC(CO)(CO)NCCS(=O)(=O)O)OCC(CO)(CO)NCCS(=O)(=O)O ; CCCCCCCCCCCCCCCCCC[N+](C)(C)CCC[Si](OCC(N)(CO)CO)(OCC([NH3+])(CO)CO)OCC([NH3+])(CO)CO]

### Embodiments of Alkyl.

In certain embodiments alkyl is a linear or branched alkyl.

In certain embodiments alkyl is C₁₋₂₂ alkyl.

In certain embodiments alkyl is C₆₋₂₂ alkyl.

In certain embodiments alkyl is C₁₋₆ alkyl.

In certain embodiments alkyl is a linear or branched C₁₋₆ alkyl.

In certain embodiments alkyl is a linear or branched C₁₋₅ alkyl.

In certain embodiments alkyl is a linear or branched C₁₋₄ alkyl.

In certain embodiments alkyl is propyl or isopropyl.

In certain embodiments alkyl is ethyl.

In certain embodiments alkyl is methyl.

### Embodiments of Haloalkyl.

In certain embodiments haloalkyl is an alkyl, wherein one or all hydrogen atoms are replaced with a halogen atom, selected from F, Cl, and Br.

In certain embodiments haloalkyl is C₁₋₆ haloalkyl.

In certain embodiments haloalkyl is a linear or branched C₁₋₆ haloalkyl.

In certain embodiments haloalkyl is a linear or branched C₁₋₅ haloalkyl.

In certain embodiments haloalkyl is a linear or branched C₁₋₄ haloalkyl.

In certain embodiments haloalkyl is CF₃.

In certain embodiments haloalkyl is CH₂CF₃.

In certain embodiments haloalkyl is CHF₂.

### Embodiments of Alkanoyl.

In certain embodiments alkanoyl is C₁₋₈ alkanoyl.

In certain embodiments alkanoyl is C₂₋₂₂ alkanoyl.

### Embodiments of Hydroxyalkyl.

In certain embodiments hydroxyalkyl is C₁₋₈ hydroxyalkyl.

In certain embodiments hydroxyalkyl is C₁₋₃ hydroxyalkyl.

### Embodiments of Alkoxy.

In certain embodiments alkoxy is C₁₋₆ alkoxy.

In certain embodiments alkoxy is a linear or branched C₁₋₆ alkoxy.

In certain embodiments alkoxy is a linear or branched C₁₋₅ alkoxy.

In certain embodiments alkoxy is a linear or branched C₁₋₄ alkoxy.

In certain embodiments alkoxy is propoxy or isopropoxy.

In certain embodiments alkoxy is ethoxy.

In certain embodiments alkoxy is methoxy.

### Embodiments of Aryl.

In certain embodiments aryl is phenyl.

### Embodiments of Cycloalkyl.

In certain embodiments cycloalkyl is C₃₋₆ cycloalkyl.

In certain embodiments cycloalkyl is cyclohexyl.

In certain embodiments cycloalkyl is cyclopentyl.

In certain embodiments cycloalkyl is cyclobutyl.

In certain embodiments cycloalkyl is cyclopropyl.

### Embodiments of Heteroaryl.

In certain embodiments heteroaryl is 5- or 6-membered heteroaryl containing 1 to 3 heteroatoms selected from O, N, and S.

In certain embodiments heteroaryl is pyridyl.

### Embodiments of Heterocycle or Heterocyclyl.

In certain embodiments heterocycle (heterocyclyl) is 3- or 7-membered saturated or unsaturated heterocycle (heterocyclyl) containing 1 to 3 heteroatoms selected from O, N, and S. In certain embodiments heterocyclyl is morpholinyl.

### Embodiments of R⁸

In certain embodiments each R⁸ is the same.

### Embodiments of R⁹

In certain embodiments each R⁹ is the same.

### Embodiments of R¹⁰

In certain embodiments each R¹⁰ is the same.

### Embodiments of R¹¹

In certain embodiments each R¹¹ is the same.

### Embodiments of R¹²

In certain embodiments each R¹² is the same.

### Embodiments of R¹³

In certain embodiments each R¹³ is the same.

### Embodiments of R¹⁶

In certain embodiments each R¹⁶ is the same.

### Embodiments of R²¹

In certain embodiments each R²¹ is the same.

Embodiments of R²², R²³, and R²⁴

In certain embodiments R²², R²³, and R²⁴ are the same.

### Embodiments of R²⁸

In certain embodiments each R²⁸ is the same.

### Embodiments of R²⁹

In certain embodiments each R²⁹ is the same.

Embodiments of R³², R³³, and R³⁴

In certain embodiments R³², R³³, and R³⁴ are the same.

### Embodiments of R⁵¹

In certain embodiments each R⁵¹ is the same.

### Embodiments of R⁵²

In certain embodiments each R⁵² is the same.

### Embodiments of R⁶⁴

In certain embodiments each R⁶⁴ is the same.

Embodiments of R⁶⁷ and R⁶⁸

In certain embodiments R⁶⁷R⁶⁸ are the same.

### Embodiments of R⁸¹

In certain embodiments each R⁸¹ is the same.

### Embodiments of R^{A}

In certain embodiments each R^{A} is the same.

### Embodiments of R^{H}

In certain embodiments each R^{H} is the same.

### Embodiments of R^{X}

In certain embodiments each R^{X} is the same.

### Embodiments of X¹

In certain embodiments each X¹ is the same.

### Embodiments of X³

In certain embodiments each X³ is the same.

### Embodiments of X⁴

In certain embodiments each X³ is the same.

### Embodiments of X¹¹

In certain embodiments each X¹¹ is the same.

### Embodiments of X²²

In certain embodiments each X²² is the same.

### Embodiments of X³¹

In certain embodiments each X³¹ is the same.

### Embodiments of X³²

In certain embodiments each X³² is the same.

### Embodiments of Z²

In certain embodiments each Z² is the same.

### Hydrolysis of Quaternary Ammonium Silanes in Hydrogel Formulation

It is believed that the compounds described herein for use in the hydrogel formulation hydrolyze in the hydrogel formulation to form a trihydroxy (quaternary ammonium) silane compound (trihydroxy-QAS) of Formula X-A:

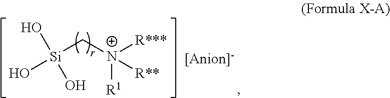

wherein
r is 1, 2, 3, 4;
R¹ is C₆-C₂₂ alkyl or C₆-C₂₂ alkanoyl;
R** and R*** are independently selected from the group consisting of:
i. C₁-C₄ alkyl; and
ii. C₆-C₂₂alkyl or C₂-C₂₂alkanoyl;
[Anion]⁻ is an anion selected from the group consisting of:
i. chloride, fluoride, iodide, bromide, hydroxide, chlorite, chlorate, hydroxide, formate, acetate, lactate, benzoate, methane sulfonate (mesylate MeSO₃⁻), and salicylate anion;
ii.

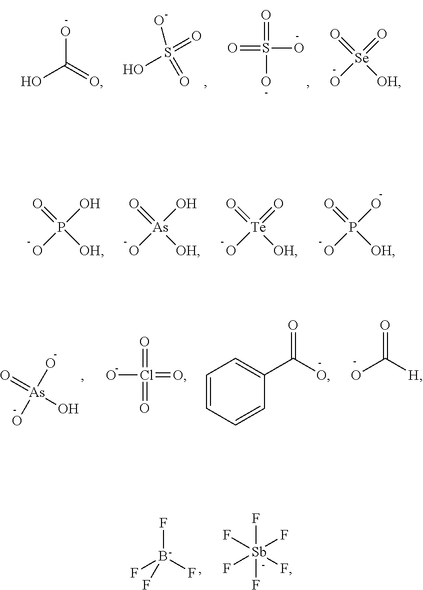

chlorite, perchlorate, hydroxide, formate, acetate, lactate, benzoate, nitrate, and salicylate anion; and
iii. an anion-containing moiety present in the compound of the invention before the hydrolysis when the compound is zwitterionic, wherein the anion-containing moiety is cleaved out from the compound as a result of the hydrolysis.

In an illustrative example of the hydrolysis of a compound of the invention, Compound XIX hydrolyzes according to the following scheme to give 2-((1,3-dihydroxy-2-(hydroxymethyl)propan-2-yl)amino)ethane-1-sulfonic acid (TES) and N,N-dimethyl-N-(3-(trihydroxysilyl)propyl)octadecan-1-aminium 2-((1,3-dihydroxy-2-(hydroxymethyl)propan-2-yl)amino)ethane-1-sulfonate:

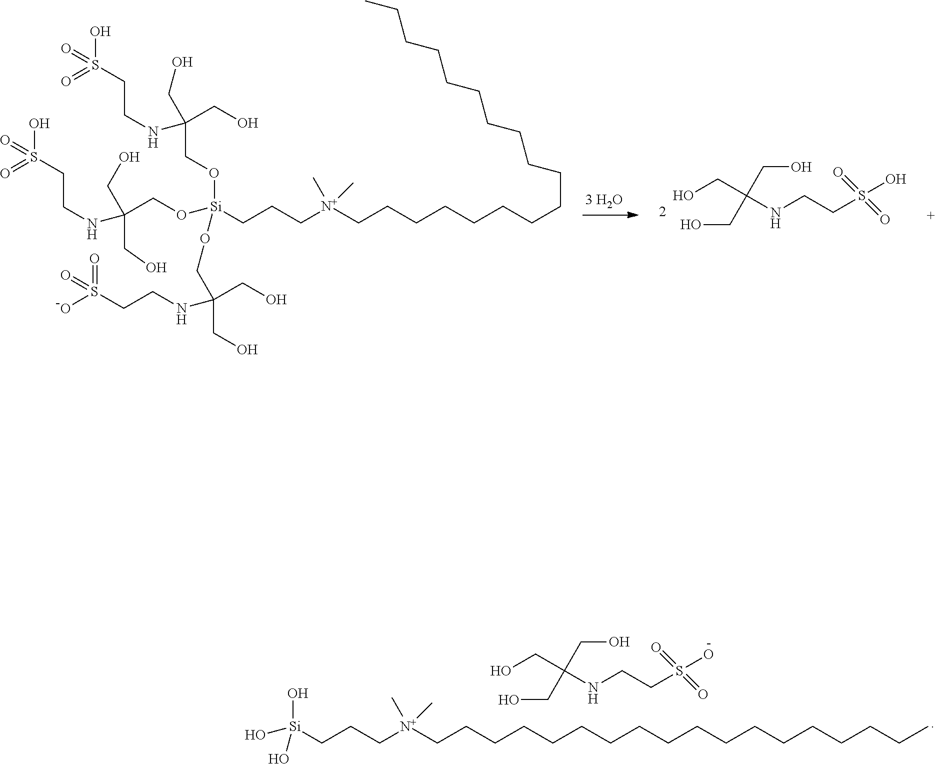 [SMILES: CCCCCCCCCCCCCCCCCC[N+](C)(C)CCC[Si](OCC(CO)(CO)NCCS(=O)(=O)O)(OCC(CO)(CO)NCCS(=O)(=O)O)OCC(CO)(CO)NCCS(=O)(=O)O ; O=S(=O)(O)CCNC(CO)(CO)CO ; CCCCCCCCCCCCCCCCCC[N+](C)(C)CCC[Si](O)(O)O ; O=S(=O)(O)CCNC(CO)(CO)CO]

In an illustrative example of the hydrolysis of a compound of the invention, Compound LIII hydrolyzes according to the following scheme to give 2-((1,3-dihydroxy-2-(hydroxymethyl)propan-2-yl)amino)ethane-1-sulfonic acid (TES) and N,N-dimethyl-N-(3-(trihydroxysilyl)propyl)octadecan-1-aminium mesylate:

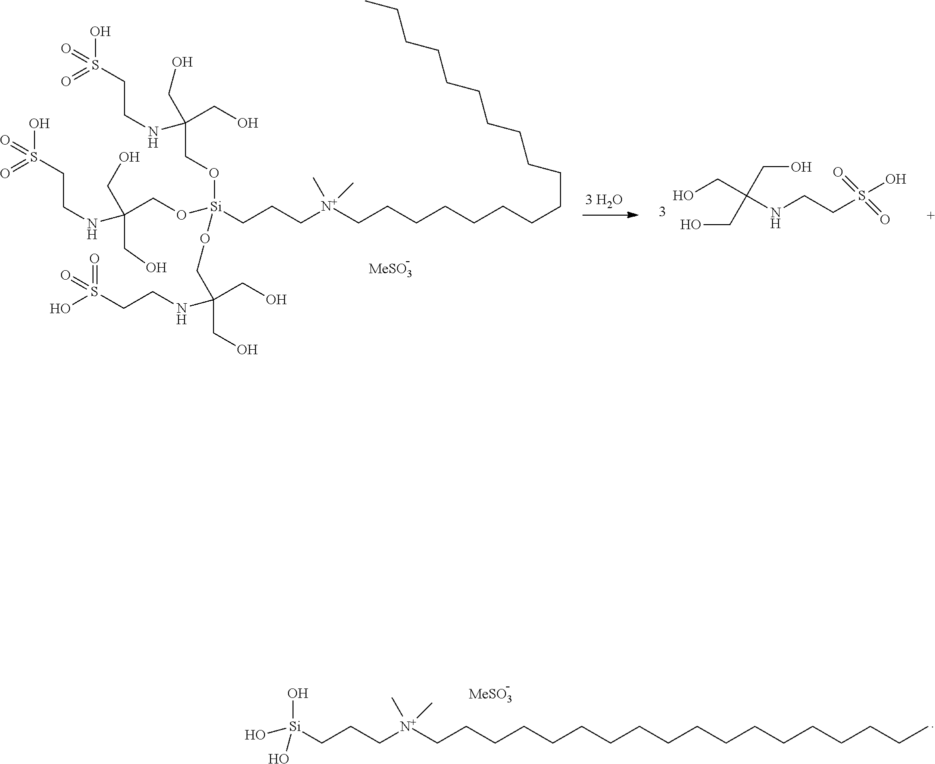 [SMILES: CCCCCCCCCCCCCCCCCC[N+](C)(C)CCC[Si](OCC(CO)(CO)NCCS(=O)(=O)O)(OCC(CO)(CO)NCCS(=O)(=O)O)OCC(CO)(CO)NCCS(=O)(=O)O ; O=S(=O)(O)CCNC(CO)(CO)CO ; CCCCCCCCCCCCCCCCCC[N+](C)(C)CCC[Si](O)(O)O]

In an illustrative example of the hydrolysis of a compound of the invention, Compound VII hydrolyzes according to the following scheme to give propylene glycol and N,N-dimethyl-N-(3-(trihydroxysilyl)propyl)octadecan-1-aminium mesylate:

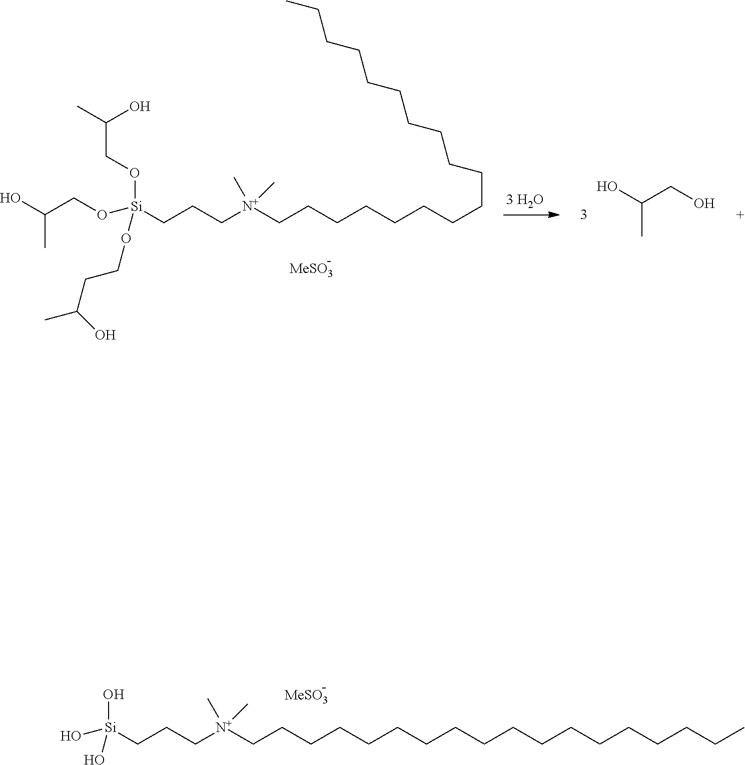

### Non-Ionic Gelling Agents

The hydrogel formulation of the present invention comprises a non-ionic gelling agent. In some embodiments, the non-ionic gelling agent lacks a carboxylic acid group. Suitable non-ionic gelling agents include, but are not limited to, hydoxypropyl methylcellulose (HPMC; also known as hypromellose), hydroxyethyl cellulose, methyl cellulose, hydroxypropyl cellulose, polydextrose, palmitostearate, methacrylated inulin, and hydroxypropyl guar.

In some embodiments, the gelling agent is hydroxypropyl methylcellulose (HPMC, hypromellose). Suitable HPMC gelling agents include, for example, but not limited to, HPMC having a viscosity of 80-120 centipose (cP), 2% in H₂O (20° C.); HPMC having an average Mn ˜10,000; HPMC having a viscosity of 40-60 cP, 2% in H₂O (20° C.); HPMC having an average Mn ˜86,000; HPMC having an average Mn ˜120,000; HPMC having a viscosity of 2,600-5,600 cP, 2% in H₂O (20° C.); HPMC having a viscosity of about 15,000 cP, 2 wt. % in H₂O (20° C.); and, HPMC having an average Mn˜90,000. In some embodiments, the HPMC used in the hydrogel formulation has a viscosity 2,600-5,600 cP, 2% in H₂O (20° C.).

In some embodiments, the quaternary ammonium silane hydrogel formulation comprises between about 0.25% and about 3.0% (w/w) of the non-ionic gelling agent. In some embodiments, the aqueous hydrogel formulation comprises between about 0.5 and about 2.5% (w/w) of the non-ionic gelling agent. In some embodiments, the aqueous hydrogel formulation about 2.0% (w/w) of the non-ionic gelling agent. In some embodiments, the quaternary ammonium silane hydrogel formulation comprises between about 0.75 and about 1.25% (w/w) of the non-ionic gelling agent. In some embodiments, the quaternary ammonium silane hydrogel formulation comprises about 1.0% (w/w) of the non-ionic gelling agent. In some embodiments, the quaternary ammonium silane hydrogel formulation comprises 0.50, 0.55, 0.60, 0.65, 0.70, 0.75, 0.80, 0.85, 0.90, 0.95, 1.0, 1.05, 1.1, 1.15, 1.2, 1.25, 1.3, 1.35, 1.4, 1.45, 1.5, 1.5, 1.7, 1.8, 1.9, 2.0, 2.1, 2.2, 2.3, 2.4, or 2.5% w/w of the gelling agent.

### Non-Ionic Adhesion Agents

The aqueous hydrogel formulations of the present invention comprise a non-ionic adhesion agent. In some embodiments, the non-ionic adhesion agent in the aqueous hydrogel formulation in the amount of between about 0.025% and about 0.5% (w/w). In some embodiments, the quaternary ammonium silane hydrogel formulation comprises between about 0.05 and about 0.2% (w/w) of the non-ionic adhesion agent. In some embodiments, the quaternary ammonium silane hydrogel formulation comprises about 0.2% (w/w) of the non-ionic adhesion agent. In some embodiments, the quaternary ammonium silane hydrogel formulation comprises between about 0.075% and about 0.15% (w/w) of the non-ionic adhesion agent. In some embodiments, the quaternary ammonium silane hydrogel formulation comprises about 0.1% (w/w) of the non-ionic adhesion agent. In some embodiments, the quaternary ammonium silane hydrogel formulation comprises 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25% w/w of the non-ionic adhesion agent.

Suitable non-ionic adhesion agents include, for example, but not limited to, a poloxamer, polyoxypropylene, polyoxyethylene, and copolymers of polyoxypropylene and polyoxyethylene. In some embodiments, the adhesion agent is a poloxamer, for example, poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate or poloxamer 182 dibenzoate. Additional non-ionic adhesion agents suitable for use in the formulations include chitosan, dextrin, glycerol monooleate, and isopropyl myristate.

In some embodiments, the adhesion agent is poloxamer 407.

### Non-Ionic Wetting Agents

The hydrogel formulations of the present invention also comprise a non-ionic wetting agent. In some embodiments, the non-ionic wetting agent lacks a carboxylic acid group. In some embodiments, the non-ionic wetting agent is present in the hydrogel formulation in an amount of between about 0.5% and about 5.0% (w/w) of a non-ionic wetting agent. In some embodiments, the quaternary ammonium silane hydrogel formulation comprises between about 1.0% and about 3.0% (w/w) non-ionic wetting agent. In some embodiments, the quaternary ammonium silane hydrogel formulation comprises between about 1.5% and about 2.5% (w/w) of the non-ionic wetting agent. In some embodiments, the quaternary ammonium silane hydrogel formulation comprises a 2.0% (w/w) of the non-ionic wetting agent. In some embodiments, the quaternary ammonium silane hydrogel formulation comprises 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, .295, or 3.0% (w/w) of the non-ionic wetting agent.

Suitable non-ionic wetting agents include, but are not limited to, propylene glycol, polypropylene glycol, glycerin, glycerol-propylene oxide copolymer, ethylene glycol, or combinations thereof. Additional suitable non-ionic wetting agents for use in the formulations also include, but are not limited to, polyoxyethylene alkyl ether, polyethylene glycol monocetyl ether, polyethylene glycol monolauryl ether, polyethylene glycol monooleyl ether, polyethylene glycol monostearyl ether, polyoxyethylene castor oil derivative, polyoxyl 5 castor oil, polyoxyl 9 castor oil, polyoxyl 15 castor oil, polyoxyl 35 castor oil, polyoxyl 40 castor oil, polyoxyl 40 hydrogenated castor oil, polyoxyl 60 castor oil, polyoxyl 60 hydrogenated castor oil, polyoxyl 100 castor oil, polyoxyl 100 hydrogenated castor oil, polyoxyl 200 castor oil, polyoxyl 200 hydrogenated castor oil, polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, polysorbate 20 (Tween 20), polysorbate 21 (Tween 21), polysorbate 40 (Tween 40), polysorbate 60 (Tween 60), polysorbate 61 (Tween 61), polysorbate 65 (Tween 65), polysorbate 80 (Tween 80), polysorbate 81 (Tween 81), polysorbate 85 (Tween 85), sorbitan ester, sorbitan fatty acid esters, sorbitan diisostearate, sorbitan dioleate, sorbitan monoisostearate, sorbitan monolaurate, sorbitan monooleate, sorbitan monopalmitate, sorbitan monostearate, sorbitan sesquiisostearate, sorbitan sesquioleate, sorbitan sesquistearate, sorbitan triisostearatesorbitan trioleate, sorbitan tristearate, and polysorbate 120 (Tween 120). In some embodiments, the wetting agent is propylene glycol. In some embodiments, the wetting agent is glycerin.

### Non-Ionic Osmotic Agents

The hydrogel formulations of the present invention optionally comprise a non-ionic osmotic agent. In some embodiments, the non-ionic osmotic is present in the hydrogel formulation in an amount of between about between about 0.5% and about 7.5% (w/w). In some embodiments, the quaternary ammonium silane hydrogel formulation comprises between about 2.0% and about 5.0% (w/w) of the non-ionic osmotic agent. In some embodiments, the quaternary ammonium silane hydrogel formulation comprises about 5.0% (w/w) of the non-ionic osmotic agent. In some embodiments, the quaternary ammonium silane hydrogel formulation comprises between about 1.0% and about 3.0% (w/w) of the non-ionic osmotic agent. In some embodiments, the quaternary ammonium silane hydrogel formulation comprises about 1.5% and about 2.5% (w/wt) of the non-ionic osmotic agent. In some embodiments, the quaternary ammonium silane hydrogel formulation comprises about 2.0% (w/w) of the non-ionic osmotic agent. In some embodiments, the quaternary ammonium silane hydrogel formulation comprises 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5% (w/w) of the non-ionic osmotic agent.

Suitable non-ionic osmotic agents include, but are not limited to, polyethylene glycol (PEG), sorbitol, lactose, lactose, fructose, dextrose, lactulose, sucrose, xylitol, mannitol, mixture of mannitol and sucrose, mixture of dextrose and fructose, mixture of sucrose and fructose, mixture of dextrose and sucrose, mixture of mannitol and fructose, mixture of lactose and fructose, mixture of mannitol and dextrose, and mixture of lactose and dextrose.

In some embodiments, the non-ionic osmotic agent is sorbitol.

In some embodiments, the non-ionic osmotic agent is polyethylene glycol (PEG). In some embodiments, the PEG has an average molecular weight (Mn) of between about 200 and 100,000. In some embodiments, the PEG has an Mn of 200, 400, 600, 1500, 2000, 3350, 4000, 6000, 8000 12000, or 20000. In some embodiments, the PEG is PEG Mn 6000.

### Aqueous Solution

The hydrogel formulations of the present invention are aqueous. Non-limiting examples of aqueous solutions that can be used include distilled water, saline, Hank's Buffered Salt Solution (HBSS), HEPES (4-(2-hydroxyethyl)-1-piperazineethanesulfonic acid), Ringers buffer, Krebs buffer, Dulbecco's PBS, normal PBS, sodium hyaluronate solution, and mixtures thereof. The solution in all instances can be sterilized in a suitable manner known to those in the art.

pH

The quaternary ammonium silane hydrogel formulations described herein preferably have a pH between about 5 and 8. In particular embodiments, the hydrogel formulation has a pH between about 5.5 and 7. In some embodiments, the pH of the hydrogel formulation is less than about 6.5. In some embodiments, the pH of the hydrogel formulation is about 6.

Accordingly, in some embodiments, the hydrogel formulation includes a pH adjusting agent which is capable of adjusting the pH of the hydrogel formulation. pH adjusting agents include, but are not limited to, methansulfonic acid, hydrochloric acid, lactic acid, levulinic acid, phosphoric acid, sodium acetate, sodium bicarbonate, sodium carbonate, sodium hydroxide, sodium lactate, and sodium phosphate. In some embodiments, the hydrogel formulation includes the pH adjusting agent methanesulfonic acid.

In some embodiments, the hydrogel formulation includes a buffer which allows the hydrogel to maintain a specific pH. Suitable buffers include, for example, but are not limited to aqueous solution of sodium acetate and acetic acid; sodium citrate and citric acid; disodium hydrogen phosphate and sodium chloride; potassium chloride and potassium dihydrogen phosphate; and boric acid and sodium tetraborate; disodium hydrogen phosphate and sodium dihydrogen phosphate; hydrochloric acid and potassium chloride.

### EPS Disrupters

In some embodiments, the aqueous hydrogel formulation optionally further comprises an agent that further disrupts and/or destabilizes some or all of the extracellular polymeric substances (EPS) that make up a biofilm or suppresses cell to cell communications, sent via ion channels in the form of electrical signals, to coordinate their behavior (an “EPS disrupter”).

In some embodiments, the EPS disruptor is selected from the group consisting of N-acetylcysteine, guaifenesin, erdosteinum, norspermidine, and a combination thereof.

In some embodiments, EPS disrupter comprises a D-amino acid, or a pharmaceutically acceptable salt, ester, or derivative thereof, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

In some embodiments, the EPS Disrupter is a proteolytic enzymes. Proteolytic enzymes may be able to act upon some of the polymeric materials present in the EPS, allowing increased penetration of the antimicrobial composition. Examples of proteolytic enzymes include, but are not limited to, collagenase, cellulase, keratinase, papain, bromelain, trypsin, thermolysin, and combinations thereof.

### Additional Agents

### Preservatives

In some embodiments, the quaternary ammonium silane hydrogel formulation optionally further comprises a preservative. While one or more quaternary ammonium silanes described herein are antimicrobial in nature, an additional preservative may be optionally included dependent on the desired shelf life of the antimicrobial composition. Examples of appropriate preservatives include, but are not limited to, benzoic acid, benzyl alcohol, chlorobutanol, chlorphenesin, dehydroacetic acid, diazolidinyl urea, ethyl lauroyl arginate HCl, ethylparaban, glyceryl caprylate, imidazolidinyl urea, methylisothiazolinone, methylparaben, o-cymen-5-ol, phenethyl alcohol, phenoxyethanol, polylysine, potassium sorbate, propylparaben, sodium anisate, sodium benzoate, sodium dehydroacetate, sodium hydroxymethylglycinate, sodium levulinate, sodium metabisulfite, sodium perborate, and sodium propionate.

### Antiseptic

In some embodiments, the aqueous hydrogel formulation optionally further comprises an additional antiseptic or antimicrobial agent. Suitable agents include, for example, but are not limited to chlorhexidine, povidone-iodine, chloroxylenol, isopropyl alcohol, hexachlorophene, benzalkonium chloride, cetylpyridinium chloride, chlorine dioxide, colostrum, dichlorobenzyl alcohol, eucalyptol, eugenol, lactoferrin, lactoperoxidase, lysozyme, octenidine dihydrochloride, thymol, hydrogen peroxide, and triclosan.

### Antioxidants

In some embodiments, the ammonium silane hydrogel formulation optionally further comprises an antioxidant. An antioxidant may be necessary to stabilize any other additive present within the antimicrobial composition from air oxidation over a suitable shelf life. Examples of appropriate antioxidants include, but are not limited to ascorbic acid, BHA, BHT, sodium bisulfite, vitamin E, sodium metabisulfite, propyl gallate, or combinations thereof.

### Demulcent Agents

In some embodiments, the aqueous hydrogel formulation optionally further comprises a demulcent agent. Suitable demulcent agents include, for example, but are not limited to, pectin.

### Plaque Carries Calculus Inhibitors

In some embodiments, the aqueous hydrogel formulation optionally further comprises a plaque controlling agent, anti-carries agent, or calculus formation inhibitor. Suitable plaque controlling agents or calculus formation inhibitors include, for example, but are not limited to, zinc gluconate, chlorhexidine digluconate (can also be used as an antiseptic), arginine, alexidine, octenidine HCl, iodine, iodophores, fluorides, calcium gluconate, bromelain, chitosan, fluorohydroxyapatite, hydroxyapatite, hypothiocyanite, Mg—Sr-carbonate hydroxyapatite conjugated with chitosan, nicotinyl alcohol HF, olaflur, papain, potassium fluoride, sodium fluoride, sodium monofluorophosphate, and stannous fluoride.

In some embodiments, the plaque controlling agent is zinc gluconate.

### Malodour Reducing Agent

In some embodiments, the aqueous hydrogel formulation optionally further comprises a malodour reducing agent. Suitable malodour reducing agents include, for example, but are not limited to, zinc acetate, zinc chloride, zinc citrate, zinc hydroxyapatite, zinc lactate, zinc PCA, and zinc sulphate.

### Chelating Agents

In some embodiments, the aqueous hydrogel formulation optionally further comprises a chelating agent. Suitable chelating agents include, for example, but are not limited to EDTA, citric acid (can also be used as a buffer), gluconic acid, pentasodium triphosphate, sodium gluconate, sodium hexametaphosphate, sodium phytate, tetrapotassium pyrophosphate, tetrasodium glutamate diacetate, and tetrasodium pyrophosphate.

### Growth Promoters

In some embodiments, the quaternary ammonium silane hydrogel formulation optionally contains a growth promoter. Growth promoters are known in the art and include, but are not limited to Platelet Derived Growth Factor (PDGF), including PDGF-BB, insulin-like growth factor (IGF), transforming growth factor-beta 1 (TGF-β1), bone morphogenetic protein (BMP), including BMP-2 and BMP-7, epidermal growth factor (EGF), fibroblast growth factor, for example FGF-2 and FGF-7, hepatocyte growth factor (HGF), vascular endothelial growth factor (VEGF), including for example VEGF-A.

### Pain Control Agent

In some embodiments, the quaternary ammonium silane hydrogel formulation optionally contains a pain control agent. Pain control agents are known in the art and include, but are not limited to, menthol, methyl salicylate (oil of evergreen), camphor, salicylates, acetaminophen, non-steroidal antiinflammatory drug including, for example, aspirin, ibuprofen, naproxen, nabumetone, a COX-2 inhibitor, for example, celecoxib, rofecoxib, valdecoxib, capsaicin, and lidocaine.

### Anti-Inflammatory Agent

In some embodiments, the quaternary ammonium silane hydrogel formulation optionally contains an anti-inflammatory agent. Anti-inflammatories are well known in the art and include, but are not limited to, NSAIDs, immune selective anti-inflammatory derivatives (ImSAIDs), antileukotrienes, and endocannabinoids.

In certain embodiments, the hydrogel formulation comprises between about 0.1% and about 5.0% (w/w) of a trihydroxy-QAS cation of Formula X that is derived from the complete hydrolysis of a compound in the hydrogel selected from the group consisting of Formula A-P and Compounds I-LIV, or a combination thereof. In certain embodiments, the hydrogel formulation comprises between about 0.1% and about 4.5% (w/w), between about 0.1% and about 4.0% (w/w), between about 0.1% and about 3.5% (w/w), between about 0.1% and about 3.0% (w/w), between about 0.1% and about 2.5% (w/w), between about 0.1% and about 2.0% (w/w), between about 0.1% and about 1.5% (w/w), between about 0.1% and about 1.0% (w/w), and between about 0.1% and about 0.5% (w/w), of a trihydroxy-QAS cation of Formula X.

In certain embodiments, the hydrogel formulation comprises between about 0.25% and about 3.0% (w/w) of a non-ionic gelling agent. In certain embodiments, the hydrogel formulation comprises between about 0.25% and about 2.8% (w/w), between about 0.25% and about 2.6% (w/w), between about 0.25% and about 2.4% (w/w), between about 0.25% and about 2.2% (w/w), between about 0.3% and about 2.2% (w/w), between about 0.4% and about 2.2% (w/w), between about 0.5% and about 2.2% (w/w), between about 0.6% and about 2.2% (w/w), between about 0.7% and about 2.2% (w/w), between about 0.8% and about 2.2% (w/w), between about 0.9% and about 2.2% (w/w), between about 1.0% and about 2.2% (w/w), between about 1.2% and about 2.2% (w/w), between about 1.4% and about 2.2% (w/w), between about 1.6% and about 2.2% (w/w), and between about 1.8% and about 2.2% (w/w) of a non-ionic gelling agent.

In certain embodiments, the hydrogel formulation comprises between about 0.025% and about 0.5% (w/w) of a non-ionic adhesion agent. In certain embodiments, the hydrogel formulation comprises between about 0.025% and about 0.40% (w/w), between about 0.05% and about 0.40% (w/w), between about 0.05% and about 0.35% (w/w), between about 0.05% and about 0.30% (w/w), between about 0.05% and about 0.25% (w/w), between about 0.10% and about 0.25% (w/w), and between about 0.15% and about 0.25% (w/w), of a non-ionic adhesion agent.

In certain embodiments, the hydrogel formulation comprises between about 0.5% and about 5.0% (w/w) of a non-ionic wetting agent. In certain embodiments, the hydrogel formulation comprises between about 1.0% and about 4.50% (w/w), between about 1.0% and about 4.0% (w/w), between about 1.0% and about 3.50% (w/w), between about 1.0% and about 3.0% (w/w), between about 1.0% and about 2.50% (w/w), between about 1.50% and about 2.50% (w/w), and between about 1.80% and about 2.2% (w/w), of a non-ionic wetting agent.

In certain embodiments, the hydrogel formulation comprises between about 0.5% and about 7.5% (w/w) of an optional non-ionic osmotic agent. In certain embodiments, the hydrogel formulation comprises between about 0.5% and about 7.0% (w/w), between about 0.5% and about 6.5% (w/w), between about 1.0% and about 6.0% (w/w), between about 1.0% and about 5.5% (w/w), between about 1.0% and about 5.0% (w/w), between about 1.5% and about 5.0% (w/w), between about 2.0% and about 5.0% (w/w), between about 2.5% and about 5.0% (w/w), between about 3.0% and about 5.0% (w/w), and between about 3.5% and about 5.0% (w/w), of an optional non-ionic osmotic agent.

In certain embodiments, the hydrogel formulation optionally comprises one or more additional agents as described herein. In certain embodiments, the hydrogel formulation optionally comprises between about 0.05% and about 5.0% (w/w) of one or more additional agents. In certain embodiments, an additional agent is a plaque controlling agent. In certain embodiments, the plaque controlling agent is zinc gluconate.

### Embodiments of Quaternary Ammonium Silane Aqueous Hydrogel Formulation

The present invention provides a stable quaternary ammonium silane aqueous hydrogel formulation comprising:
a) a compound selected from the group consisting of Formula A-P and Compounds I-LIV, wherein the compound is present in the hydrogel formulation at a concentration that results in a trihydroxy-QAS cation present in the hydrogel at between about 0.1% and 5.0% (weight/weight) (w/w) when the compound is fully hydrolyzed, wherein the trihydroxy-QAS cation is of formula

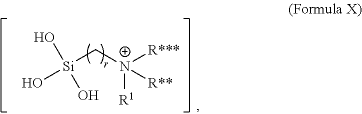

wherein
   r is 1, 2, 3, or 4;
   R¹ is C₆-C₂₂alkyl or C₆-C₂₂ alkanoyl;
   R** and R*** are independently selected from the group consisting of:
   i. C₁-C₄ alkyl; and
   ii. C₆-C₂₂alkyl or C₂-C₂₂alkanoyl;
b) between about 0.25% and about 3.0% (w/w) of a non-ionic gelling agent;
c) between about 0.025% and about 0.5% (w/w) of a non-ionic adhesion agent;
d) between about 0.5% and about 5.0% (w/w) of a non-ionic wetting agent;
e) optionally between about 0.5% and about 7.5% (w/w) of a non-ionic osmotic agent; and,
f) optionally one or more additional agents as described further herein.

In certain embodiments, the aqueous hydrogel formulation comprises:
a) between about 0.25% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.5% and about 2.5% (w/w) of a non-ionic gelling agent;
c) between about 0.05% to and about 0.4% (w/w) of a non-ionic adhesion agent;
d) between about 1.0% and about 3.0% (w/w) of a non-ionic wetting agent;
e) between about 0.5% and about 6.0% (w/w) of a non-ionic osmotic agent; and
f) between about 0.05% and about 1.0% (w/w) of one or two additional agents.

In certain embodiments, the aqueous hydrogel formulation comprises:
a) between about 0.25% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.5% and about 2.5% (w/w) of a non-ionic gelling agent;
c) between about 0.05% to and about 0.4% (w/w) of a non-ionic adhesion agent;
d) between about 1.0% and about 3.0% (w/w) of a non-ionic wetting agent;
e) between about 0.5% and about 6.0% (w/w) of a non-ionic osmotic agent; and
f) between about 0.05% and about 1.0% (w/w) of an additional agent.

In certain embodiments, the aqueous hydrogel formulation comprises:
a) between about 0.25% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.7% and about 2.5% (w/w) of a non-ionic gelling agent;
c) between about 0.1% to and about 0.3% (w/w) of a non-ionic adhesion agent;
d) between about 1.0% and about 3.0% (w/w) of a non-ionic wetting agent;
e) between about 1.0% and about 6.0% (w/w) of a non-ionic osmotic agent; and
f) between about 0.05% and about 1.0% (w/w) of an additional agent.

In certain embodiments, the aqueous hydrogel formulation comprises:
a) between about 0.25% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.7% and about 2.5% (w/w) of a non-ionic gelling agent;
c) between about 0.1% to and about 0.3% (w/w) of a non-ionic adhesion agent;
d) between about 1.0% and about 3.0% (w/w) of a non-ionic wetting agent; and
e) between about 0.05% and about 1.0% (w/w) of an additional agent.

In certain embodiments, the aqueous hydrogel formulation comprises:
a) between about 0.3% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.8% and about 2.5% (w/w) of a non-ionic gelling agent;
c) between about 0.1% to and about 0.3% (w/w) of a non-ionic adhesion agent;
d) between about 1.2% and about 3.0% (w/w) of a non-ionic wetting agent;
e) between about 1.5% and about 6.0% (w/w) of a non-ionic osmotic agent; and
f) between about 0.05% and about 1.0% (w/w) of an additional agent.

In certain embodiments, the aqueous hydrogel formulation comprises:
a) between about 0.3% and about 1.0% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 0.8% and about 2.5% (w/w) of a non-ionic gelling agent;
c) between about 0.1% to and about 0.3% (w/w) of a non-ionic adhesion agent;
d) between about 1.2% and about 3.0% (w/w) of a non-ionic wetting agent; and
e) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.3% (w/w) of a non-ionic gelling agent;
c) between about 0.1% to and about 0.3% (w/w) of a non-ionic adhesion agent;
d) between about 1.5% and about 3.0% (w/w) of a non-ionic wetting agent;
e) between about 2.0% and about 6.0% (w/w) of a non-ionic osmotic agent; and
f) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.3% (w/w) of a non-ionic gelling agent;
c) between about 0.1% to and about 0.3% (w/w) of a non-ionic adhesion agent;
d) between about 1.5% and about 3.0% (w/w) of a non-ionic wetting agent; and
e) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.1% to and about 0.3% (w/w) of a non-ionic adhesion agent;
d) between about 1.7% and about 2.5% (w/w) of a non-ionic wetting agent;
e) between about 2.5% and about 6.0% (w/w) of a non-ionic osmotic agent; and
f) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.1% to and about 0.3% (w/w) of a non-ionic adhesion agent;
d) between about 1.7% and about 2.5% (w/w) of a non-ionic wetting agent; and
e) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 3.0% and about 6.0% (w/w) of a non-ionic osmotic agent; and
f) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent; and
e) between about 0.05% and about 1.0% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 3.5% and about 6.0% (w/w) of a non-ionic osmotic agent; and
f) between about 0.05% and about 0.8% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.5% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 4.0% and about 5.5% (w/w) of a non-ionic osmotic agent; and
f) between about 0.05% and about 0.6% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 4.0% and about 5.5% (w/w) of a non-ionic osmotic agent; and
f) between about 0.05% and about 0.4% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.0% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent; and
e) between about 0.05% and about 0.4% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b) between about 1.5% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent; and
e) between about 0.05% and about 0.2% (w/w) of an additional agent.

In certain embodiments, the aqueous hydrogel formulation comprises:
a) between about 0.4% and about 1.0% (w/w) of a trihydroxy-QAS cation of Formula X that is derived from the complete hydrolysis of Compound XIX;
b) between about 1.5% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 4.0% and about 5.5% (w/w) of a non-ionic osmotic agent; and
f) between about 0.05% and about 0.4% (w/w) of an additional agent.

In certain embodiments, the aqueous hydrogel formulation comprises:
a) between about 0.4% and about 1.0% (w/w) of a trihydroxy-QAS cation of Formula X that is derived from the complete hydrolysis of Compound XIX;
b) between about 1.5% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 0.05% and about 0.4% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of Compound XIX;
b) between about 1.5% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 4.0% and about 5.5% (w/w) of a non-ionic osmotic agent; and
f) between about 0.05% and about 0.4% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of Compound XIX;
b) between about 1.5% and about 2.2% (w/w) of a non-ionic gelling agent;
c) between about 0.15% to and about 0.25% (w/w) of a non-ionic adhesion agent;
d) between about 1.7% and about 2.3% (w/w) of a non-ionic wetting agent;
e) between about 0.05% and about 0.4% (w/w) of an additional agent.

In certain embodiments, the aqueous hydrogel formulation comprises:
a) between about 0.3% and about 0.8% (w/w) of a trihydroxy-QAS cation of Formula X that is derived from the complete hydrolysis of Compound XIX;
b) between about 1.5% and about 2.2% (w/w) of HPMC;
c) between about 0.15% to and about 0.25% (w/w) of Poloxamer 407;
d) between about 1.7% and about 2.3% (w/w) of glycerin;
e) between about 4.0% and about 5.5% (w/w) of sorbitol; and
f) between about 0.05% and about 0.4% (w/w) of zinc gluconate.

In certain embodiments, the aqueous hydrogel formulation comprises:
a) between about 0.3% and about 0.8% (w/w) of a trihydroxy-QAS cation of Formula X that is derived from the complete hydrolysis of Compound XIX;
b) between about 1.5% and about 2.2% (w/w) of HPMC;
c) between about 0.15% to and about 0.25% (w/w) of Poloxamer 407;
d) between about 1.7% and about 2.3% (w/w) of glycerin; and
e) between about 0.05% and about 0.4% (w/w) of zinc gluconate.

In certain embodiments, the aqueous hydrogel formulation comprises:
a) between about 0.4% and about 1.0% (w/w) of a trihydroxy-QAS cation of Formula X that is derived from the complete hydrolysis of Compound XIX;
b) between about 1.5% and about 2.2% (w/w) of HPMC;
c) between about 0.15% to and about 0.25% (w/w) of Poloxamer 407;
d) between about 1.7% and about 2.3% (w/w) of glycerin;
e) between about 4.0% and about 5.5% (w/w) of sorbitol; and
f) between about 0.05% and about 0.4% (w/w) of zinc gluconate.

In certain embodiments, the aqueous hydrogel formulation comprises:
a) between about 0.4% and about 1.0% (w/w) of a trihydroxy-QAS cation of Formula X that is derived from the complete hydrolysis of Compound XIX;
b) between about 1.5% and about 2.2% (w/w) of HPMC;
c) between about 0.15% to and about 0.25% (w/w) of Poloxamer 407;
d) between about 1.7% and about 2.3% (w/w) of glycerin; and
e) between about 0.05% and about 0.4% (w/w) of zinc gluconate.

In certain embodiments, the aqueous hydrogel formulation comprises:
a) between about 0.4% and about 0.6% (w/w) of a trihydroxy-QAS cation of Formula X that is derived from the complete hydrolysis of Compound XIX;
b) between about 1.8% and about 2.2% (w/w) of HPMC;
c) between about 0.15% to and about 0.25% (w/w) of Poloxamer 407;
d) between about 1.7% and about 2.3% (w/w) of glycerin;
e) between about 4.5% and about 5.5% (w/w) of sorbitol; and
f) between about 0.05% and about 0.2% (w/w) of zinc gluconate.

In certain embodiments, the aqueous hydrogel formulation comprises:
a) between about 0.4% and about 0.6% (w/w) of a trihydroxy-QAS cation of Formula X that is derived from the complete hydrolysis of Compound XIX;
b) between about 1.8% and about 2.2% (w/w) of HPMC;
c) between about 0.15% to and about 0.25% (w/w) of Poloxamer 407;
d) between about 1.7% and about 2.3% (w/w) of glycerin; and
e) between about 0.05% and about 0.2% (w/w) of zinc gluconate.

### Embodiments of the Invention

Embodiment 1. A quaternary ammonium silane aqueous hydrogel formulation comprising:
a) a quaternary ammonium silane compound selected from the group consisting of Formula A-P and Compounds I-LIV;
b) a non-ionic gelling agent;
c) a non-ionic adhesion agent;
d) a non-ionic wetting agent;
e) optionally a non-ionic osmotic agent; and,
f) an aqueous solution.

Embodiment 2. The aqueous hydrogel formulation according to embodiment 1 comprising: a compound selected from the group consisting of Formula A-P and Compounds I-a) LIV, wherein the compound is present in the hydrogel formulation at a concentration that results in a trihydroxy-QAS cation present in the hydrogel at between about 0.1% and 5.0% (weight/weight) (w/w) when the compound is fully hydrolyzed, wherein the trihydroxy-QAS cation is of formula

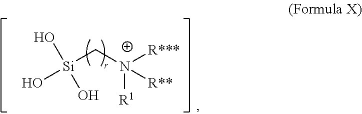

wherein
   r is 1, 2, 3, or 4;
   R¹ is C₆-C₂₂alkyl or C₆-C₂₂ alkanoyl;
   R** and R*** are independently selected from the group consisting of:
   i. C₁-C₄ alkyl; and,
   ii. C₆-C₂₂alkyl or C₂-C₂₂alkanoyl;
b) between about 0.25% and about 3.0% (w/w) of a non-ionic gelling agent;
c) between about 0.025% and about 0.5% (w/w) of a non-ionic adhesion agent;
d) between about 0.5% and about 5.0% (w/w) of a non-ionic wetting agent; and,
e) optionally between about 0.5% and about 7.5% (w/w) of a non-ionic osmotic agent.

Embodiment 3. The aqueous hydrogel formulation of embodiment 1 or 2, wherein the trihydroxy-QAS cation is of structure:

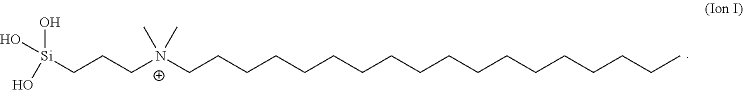 [SMILES: CCCCCCCCCCCCCCCCCC[N+](C)(C)CCC[Si](O)(O)O]

Embodiment 4. The aqueous hydrogel formulation of any one of embodiments 1-3, wherein the non-ionic gelling agent is present in a concentration of about 2% (w/w).

Embodiment 5. The aqueous hydrogel formulation of any one of embodiments 1-3, wherein the non-ionic gelling agent is present in a concentration of between about 0.5% (w/w) and 1.5% (w/w).

Embodiment 6. The aqueous hydrogel formulation of any one of embodiments 1-3, wherein the non-ionic gelling agent is present in a concentration of between about 0.75% (w/w) and about 1.25% (w/w).

Embodiment 7. The aqueous hydrogel formulation of any one of embodiments 1-3, wherein the non-ionic gelling agent is present in a concentration of about 1% (w/w).

Embodiment 8. The aqueous hydrogel formulation of any one of embodiments 1-7, wherein the non-ionic gelling agent lacks a carboxylic acid group.

Embodiment 9. The aqueous hydrogel formulation of any one of embodiments 1-8, wherein the non-ionic gelling agent is selected from the group consisting of hydoxypropyl methylcellulose (HPMC), hydroxyethyl cellulose, methyl cellulose, hydroxypropyl cellulose, polydextrose, palmitostearate, methacrylated inulin, and hydroxypropyl guar.

Embodiment 10. The aqueous hydrogel formulation of embodiment 9, wherein the non-ionic gelling agent is HPMC.

Embodiment 11. The aqueous hydrogel formulation of embodiment 10, wherein the HPMC has a viscosity of between about 2600 and 5600 cP at 2% in water at 20° C.

Embodiment 12. The hydrogel formulation of any one of embodiments 1-11, wherein the non-ionic adhesion agent is present in the amount of between about 0.025% (w/w) and about 0.5% (w/w).

Embodiment 13. The hydrogel formulation of any one of embodiments 1-12, wherein the non-ionic adhesion agent is present in the amount of between about 0.05% (w/w) and about 0.25% (w/w).

Embodiment 14. The hydrogel formulation of embodiment 13, wherein the non-ionic adhesion agent is present in the amount of about 0.2% (w/w).

Embodiment 15. The hydrogel formulation of any one of embodiments 1-12, wherein the non-ionic adhesion agent is present in the amount of between about 0.075% (w/w) and about 0.15% (w/w).

Embodiment 16. The hydrogel formulation of embodiment 15, wherein the non-ionic adhesion agent is present in the amount of about 0.1% (w/w).

Embodiment 17. The hydrogel formulation of any one of embodiments 1-16, wherein the non-ionic adhesion agent is selected from the group consisting of a poloxamer, polyoxypropylene, polyoxyethylene, and copolymers of polyoxypropylene and polyoxyethylene.

Embodiment 18. The hydrogel formulation of embodiment 17, wherein the non-ionic adhesion agent is a poloxamer.

Embodiment 19. The hydrogel formulation of embodiment 18, wherein the non-ionic adhesion agent is poloxamer 407.

Embodiment 20. The hydrogel formulation of any one of embodiments 1-19, wherein the non-ionic wetting agent is present in a concentration of between about 1.0% (w/w) and 3.0% (w/w).

Embodiment 21. The hydrogel formulation of any one of embodiments 1-20, wherein the non-ionic wetting agent is present in a concentration of between about 1.5% and about 2.5% (w/w).

Embodiment 22. The hydrogel formulation of embodiment 21, wherein the non-ionic wetting agent is present in a concentration of about 2.0% (w/w).

Embodiment 23. The hydrogel formulation of any one of embodiments 1-22, wherein the non-ionic wetting agent lacks a carboxylic acid group.

Embodiment 24. The hydrogel formulation of any one of embodiments 1-23, wherein the non-ionic wetting agent is selected from the group consisting of propylene glycol, polypropylene glycol, glycerin, glycerol, glycerol-propylene oxide copolymer, ethylene glycol, polyvinyl alcohol, and combinations thereof.

Embodiment 25. The hydrogel formulation of embodiment 24, wherein the non-ionic wetting agent is propylene glycol.

Embodiment 26. The hydrogel formulation of embodiment 24, wherein the non-ionic wetting agent is glycerine.

Embodiment 27. The hydrogel formulation of any one of embodiments 1-26, wherein the hydrogel formulation further comprises a non-ionic osmotic agent.

Embodiment 28. The hydrogel formulation of any one of embodiments 1-27, wherein the non-ionic osmotic agent is present in the hydrogel at a concentration of about 5.0% (w/w).

Embodiment 29. The hydrogel formulation of any one of embodiments 1-27, wherein the non-ionic osmotic agent is present in the hydrogel at a concentration of between about 1.0% (w/w) and about 3.0% (w/w).

Embodiment 30. The hydrogel formulation of any one of embodiments 1-27, wherein the non-ionic osmotic agent is present in the hydrogel at a concentration of between about 1.0% (w/w) and about 3.0% (w/w).

Embodiment 31. The hydrogel formulation of embodiment 30, wherein the non-ionic osmotic agent is present in the hydrogel at a concentration of about 2.0% (w/w).

Embodiment 32. The hydrogel formulation of any one of embodiments 1-31, wherein the non-ionic osmotic agent is selected from the group consisting of polyethylene glycol (PEG), sorbitol, lactose, fructose, dextrose, lactulose, sucrose, xylitol, mannitol, and mixtures thereof.

Embodiment 33. The hydrogel formulation of any one of embodiments 1-32, wherein the non-ionic osmotic agent is PEG.

Embodiment 34. The hydrogel formulation of embodiment 33, wherein the PEG has an average molecular weight (Mn) of between about 200 and about 100,000.

Embodiment 35. The hydrogel formulation of embodiment 34, wherein the PEG has an average Mn of 6000.

Embodiment 36. The hydrogel formulation of any one of embodiments 1-32, wherein the non-ionic osmotic agent is sorbitol.

Embodiment 37. The hydrogel formulation of any one of embodiments 1-36, wherein the aqueous solution is selected from the group of water, saline, or a phosphate buffered saline.

Embodiment 38. The hydrogel formulation of any one of embodiments 37, wherein the aqueous solution is water.

Embodiment 39. The hydrogel formulation of any one of embodiments 1-37, wherein the hydrogel comprises between about 0.25% and about 3% (w/w) trihydroxy-QAS cation.

Embodiment 40. The hydrogel formulation of any one of embodiments 1-37, wherein the hydrogel comprises between about 0.25% and 1.0% (w/w) trihydroxy-QAS cation.

Embodiment 41. The hydrogel formulation of any one of embodiments 1-37, wherein the hydrogel comprises between about 0.5% and about 0.75% (w/w) trihydroxy-QAS cation.

Embodiment 42. The hydrogel formulation of any one of embodiments 1-37, wherein the hydrogel comprises about 0.5% (w/w) trihydroxy-QAS cation.

Embodiment 43. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 2.0% (w/w) of HPMC;
c. about 0.1% (w/w) of poloxamer 407;
d. about 2.0% (w/w) of propylene glycol;
e. about 2.0% (w/w) of PEG; and,
f. optionally one or more additional agents.

Embodiment 44. The aqueous formulation of embodiment 43, wherein the PEG has a Mn of 6000.

Embodiment 45. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 1.0% (w/w) of HPMC;
c. about 0.1% (w/w) of poloxamer 407;
d. about 2.0% (w/w) of propylene glycol;
e. about 2.0% (w/w) of PEG; and,
f. optionally one or more additional agents.

Embodiment 46. The aqueous formulation of embodiment 45, wherein the PEG has a Mn of 6000.

Embodiment 47. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 2.0% (w/w) of HPMC;
c. about 0.2% (w/w) of poloxamer 407;
d. about 2.0% (w/w) of glycerin;
e. about 5.0% (w/w) of a sorbitol; and,
f. optionally one or more additional agents.

Embodiment 48. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 1.0% (w/w) of HPMC;
c. about 0.2% (w/w) of poloxamer 407;
d. about 2.0% (w/w) of glycerin;
e. about 5.0% (w/w) of a sorbitol; and,
f. optionally one or more additional agents.

Embodiment 49. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 2.0% (w/w) of HPMC;
c. about 0.2% (w/w) of poloxamer 407;
d. about 2.0% (w/w) of glycerin; and,
e. optionally one or more additional agents.

Embodiment 50. The aqueous hydrogel formulation of any one of embodiments 45-49, wherein the HPMC has a viscosity of between about 2600 and 5600 cP at 2% in water at 20° C.

Embodiment 51. The aqueous hydrogel formulation of any one of embodiments 1-50, wherein the hydrogel formulation has a pH between about 5 and about 8.

Embodiment 52. The aqueous hydrogel formulation of any one of embodiments 1-50, wherein the hydrogel formulation has a pH between about 5.5 and about 6.5.

Embodiment 53. The aqueous hydrogel formulation of any one of embodiments 1-50, wherein the hydrogel formulation has a a pH of about 6.

Embodiment 54. The aqueous hydrogel formulation of any one of embodiments 1-53, wherein the hydrogel formulation further comprises a pH adjusting agent.

Embodiment 55. The aqueous hydrogel formulation of embodiment 54, wherein the pH adjusting agent is methanesulfonic acid.

Embodiment 56. The aqueous hydrogel formulation of any one of embodiments 1-55, wherein the hydrogel formulation further comprises a buffer.

Embodiment 57. The aqueous hydrogel formulation of any one of embodiments 1-56, wherein the hydrogel formulation has a viscosity of between about 1000 and 2000 cP.

Embodiment 58. The aqueous hydrogel formulation of any one of embodiments 1-56, wherein the hydrogel formulation has a viscosity of between about 1250 and 1600 cP.

Embodiment 59. The aqueous hydrogel formulation of any one of embodiments 1-56, wherein the hydrogel formulation has a viscosity of about 1600 cP.

Embodiment 60. The aqueous hydrogel formulation of any one of embodiments 1-56, wherein the hydrogel formulation has a viscosity of about 1250 cP.

Embodiment 61. The aqueous hydrogel formulation of any one of embodiments 1-60, wherein the hydrogel formulation further comprises an agent that disrupts and/or destabilizes the extracellular polymeric substances (EPS) that make up a biofilm (an “EPS disrupter”).

Embodiment 62. The aqueous hydrogel formulation of embodiment 61, wherein the EPS disrupter is selected from the group consisting of N-acetylcysteine, guaifenesin, erdosteinum, norspermidine, and a combination thereof.

Embodiment 63. The aqueous hydrogel formulation of embodiment 61, wherein the EPS disrupter is a D-amino acid, or a pharmaceutically acceptable salt, ester, or derivative thereof.

Embodiment 64. The aqueous hydrogel formulation of embodiment 63, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

Embodiment 65. The aqueous hydrogel formulation of embodiment 61, wherein the EPS disrupter is a proteolytic enzyme.

Embodiment 66. The aqueous hydrogel formulation of embodiment 65, wherein the proteolytic enzyme is selected from the group consisting of collagenase, cellulase, keratinase, papain, bromelain, trypsin, thermolysin, and combinations thereof.

Embodiment 67. The aqueous hydrogel formulation of any one of embodiments 1-66, wherein the hydrogel formulation further comprises a plaque controlling agent.

Embodiment 68. The aqueous hydrogel formulation of embodiment 67, wherein the plaque controlling agent is selected from the group consisting of zinc gluconate, chlorhexidine digluconate, arginine, alexidine, octenidine HCl, iodine, iodophores, fluorides, calcium gluconate, bromelain, chitosan, fluorohydroxyapatite, hydroxyapatite, hypothiocyanite, Mg—Sr-carbonate hydroxyapatite conjugated with chitosan, nicotinyl alcohol HF, olaflur, papain, potassium fluoride, sodium fluoride, sodium monofluorophosphate, and stannous fluoride.

Embodiment 69. The aqueous hydrogel formulation of embodiment 68, wherein the plaque controlling agent is zinc gluconate.

Embodiment 70. The aqueous hydrogel formulation of any one of embodiments 1-69, wherein the compound is a compound of Formula A.

Embodiment 71. The aqueous hydrogel formulation of any one of embodiments 1-69, wherein the compound is a compound of Formula A-1.

Embodiment 72. The aqueous hydrogel formulation of any one of embodiments 1-69, wherein the compound is a compound of Formula A-2.

Embodiment 73. The aqueous hydrogel formulation of any one of embodiments 1-69, wherein the compound is a compound of Formula A-3.

Embodiment 74. The aqueous hydrogel formulation of any one of embodiments 1-69, wherein the compound is a compound of Formula B.

Embodiment 75. The aqueous hydrogel formulation of any one of embodiments 1-63, wherein the compound is a compound of Formula B-1.

Embodiment 76. The aqueous hydrogel formulation of any one of embodiments 1-69, wherein the compound is a compound of Formula B-2.

Embodiment 77. The aqueous hydrogel formulation of any one of embodiments 1-69, wherein the compound is a compound of Formula B-3.

Embodiment 78. The aqueous hydrogel formulation of any one of embodiments 1-69, wherein the compound is Compound I.

Embodiment 79. The aqueous hydrogel formulation of any one of embodiments 1-69, wherein the compound is Compound II.

Embodiment 80. The aqueous hydrogel formulation of any one of embodiments 1-69, wherein the compound is Compound III.

Embodiment 81. The aqueous hydrogel formulation of any one of embodiments 1-69, wherein the compound is Compound IV.

Embodiment 82. The aqueous hydrogel formulation of any one of embodiments 1-69, wherein the compound is Compound V.

Embodiment 83. The aqueous hydrogel formulation of any one of embodiments 1-69, wherein the compound is Compound VI.

Embodiment 84. The aqueous hydrogel formulation of any one of embodiments 1-69, wherein the compound is Compound VII.

Embodiment 85. The aqueous hydrogel formulation of any one of embodiments 1-69, wherein the compound is Compound VIII.

Embodiment 86. The aqueous hydrogel formulation of any one of embodiments 1-69, wherein the compound is Compound IX.

Embodiment 87. The aqueous hydrogel formulation of any one of embodiments 1-69, wherein the compound is Compound X.

Embodiment 88. The aqueous hydrogel formulation of any one of embodiments 1-69, wherein the compound is Compound XI.

Embodiment 89. The aqueous hydrogel formulation of any one of embodiments 1-69, wherein the compound is Compound XII.

Embodiment 90. The aqueous hydrogel formulation of any one of embodiments 1-69, wherein the compound is Compound XIII.

Embodiment 91. The aqueous hydrogel formulation of any one of embodiments 1-69, wherein the compound is Compound XIV.

Embodiment 92. The aqueous hydrogel formulation of any one of embodiments 1-69, wherein the compound is Compound XV.

Embodiment 93. The aqueous hydrogel formulation of any one of embodiments 1-69, wherein the compound is Compound XVI.

Embodiment 94. The aqueous hydrogel formulation of any one of embodiments 1-69, wherein the compound is Compound XVII.

Embodiment 95. The aqueous hydrogel formulation of any one of embodiments 1-69, wherein the compound is Compound XIII.

Embodiment 96. The aqueous hydrogel formulation of any one of embodiments 1-69, wherein the compound is a compound of Formula C.

Embodiment 97. The aqueous hydrogel formulation of any one of embodiments 1-69, wherein the compound is a compound of Formula D.

Embodiment 98. The aqueous hydrogel formulation of any one of embodiments 1-69, wherein the compound is Compound XIX.

Embodiment 99. The aqueous hydrogel formulation of any one of embodiments 1-69, wherein the compound is Compound XX.

Embodiment 100. The aqueous hydrogel formulation of any one of embodiments 1-69, wherein the compound is Compound XXI.

Embodiment 101. The aqueous hydrogel formulation of any one of embodiments 1-69, wherein the compound is Compound XXII.

Embodiment 102. The aqueous hydrogel formulation of any one of embodiments 1-69, wherein the compound is Compound XXIII.

Embodiment 103. The aqueous hydrogel formulation of any one of embodiments 1-69, wherein the compound is Compound XIV.

Embodiment 104. The aqueous hydrogel formulation of any one of embodiments 1-69, wherein the compound is Compound XXV.

Embodiment 105. The aqueous hydrogel formulation of any one of embodiments 1-69, wherein the compound is a compound of Formula E.

Embodiment 106. The aqueous hydrogel formulation of any one of embodiments 1-69, wherein the compound is a compound of Formula F.

Embodiment 107. The aqueous hydrogel formulation of any one of embodiments 1-69, wherein the compound is a compound of Compound XXVI.

Embodiment 108. The aqueous hydrogel formulation of any one of embodiments 1-69, wherein the compound is a compound of Compound XXVII.

Embodiment 109. The aqueous hydrogel formulation of any one of embodiments 1-69, wherein the compound is a compound of Compound XXVIII.

Embodiment 110. The aqueous hydrogel formulation of any one of embodiments 1-69, wherein the compound is a compound of Compound XXIX.

Embodiment 111. The aqueous hydrogel formulation of any one of embodiments 1-69, wherein the compound is a compound of Compound XXX.

Embodiment 112. The aqueous hydrogel formulation of any one of embodiments 1-69, wherein the compound is a compound of Compound XXXI.

Embodiment 113. The aqueous hydrogel formulation of any one of embodiments 1-69, wherein the compound is a compound of Formula G.

Embodiment 114. The aqueous hydrogel formulation of any one of embodiments 1-69, wherein the compound is a compound of Formula H.

Embodiment 115. The aqueous hydrogel formulation of any one of embodiments 1-69, wherein the compound is a compound of Formula I.

Embodiment 116. The aqueous hydrogel formulation of any one of embodiments 1-69, wherein the compound is a compound of Formula J.

Embodiment 117. The aqueous hydrogel formulation of any one of embodiments 1-69, wherein the compound is a compound of Formula K.

Embodiment 118. The aqueous hydrogel formulation of any one of embodiments 1-69, wherein the compound is a compound of Compound XXXII.

Embodiment 119. The aqueous hydrogel formulation of any one of embodiments 1-69, wherein the compound is a compound of Compound XXXIII.

Embodiment 120. The aqueous hydrogel formulation of any one of embodiments 1-69, wherein the compound is a compound of Compound XXXIV.

Embodiment 121. The aqueous hydrogel formulation of any one of embodiments 1-69, wherein the compound is a compound of Compound XXXV.

Embodiment 122. The aqueous hydrogel formulation of any one of embodiments 1-69, wherein the compound is a compound of Compound XXXVI.

Embodiment 123. The aqueous hydrogel formulation of any one of embodiments 1-69, wherein the compound is a compound of Compound XXXVII.

Embodiment 124. The aqueous hydrogel formulation of any one of embodiments 1-69, wherein the compound is a compound of Compound XXXVIII.

Embodiment 125. The aqueous hydrogel formulation of any one of embodiments 1-69, wherein the compound is a compound of Compound XXXIX.

Embodiment 126. The aqueous hydrogel formulation of any one of embodiments 1-69, wherein the compound is a compound of Compound XL.

Embodiment 127. The aqueous hydrogel formulation of any one of embodiments 1-69, wherein the compound is a compound of Compound XLI.

Embodiment 128. The aqueous hydrogel formulation of any one of embodiments 1-69, wherein the compound is a compound of Compound XLII.

Embodiment 129. The aqueous hydrogel formulation of any one of embodiments 1-69, wherein the compound is a compound of Compound XLIII.

Embodiment 130. The aqueous hydrogel formulation of any one of embodiments 1-69, wherein the compound is a compound of Compound XLIV.

Embodiment 131. The aqueous hydrogel formulation of any one of embodiments 1-69, wherein the compound is a compound of Compound XLV.

Embodiment 132. The aqueous hydrogel formulation of any one of embodiments 1-69, wherein the compound is a compound of Compound XL VI.

Embodiment 133. The aqueous hydrogel formulation of any one of embodiments 1-69, wherein the compound is a compound of Compound XL VII.

Embodiment 134. The aqueous hydrogel formulation of any one of embodiments 1-69, wherein the compound is a compound of Formula L.

Embodiment 135. The aqueous hydrogel formulation of any one of embodiments 1-69, wherein the compound is a compound of Formula M.

Embodiment 136. The aqueous hydrogel formulation of any one of embodiments 1-69, wherein the compound is a compound of Formula N.

Embodiment 137. The aqueous hydrogel formulation of any one of embodiments 1-69, wherein the compound is Compound XL VIII.

Embodiment 138. The aqueous hydrogel formulation of any one of embodiments 1-69, wherein the compound is Compound XLIX.

Embodiment 139. The aqueous hydrogel formulation of any one of embodiments 1-69, wherein the compound is Compound L.

Embodiment 140. The aqueous hydrogel formulation of any one of embodiments 1-69, wherein the compound is Compound LI.

Embodiment 141. The aqueous hydrogel formulation of any one of embodiments 1-69, wherein the compound is Compound LII.

Embodiment 142. The aqueous hydrogel formulation of any one of embodiments 1-69, wherein the compound is a compound of Formula O.

Embodiment 143. The aqueous hydrogel formulation of any one of embodiments 1-69, wherein the compound is a compound of Formula P.

Embodiment 144. The aqueous hydrogel formulation of any one of embodiments 1-69, wherein the compound is Compound LIII.

Embodiment 145. The aqueous hydrogel formulation of any one of embodiments 1-69, wherein the compound is Compound LIV.

Embodiment 146. A method of inhibiting the detachment and/or dissemination of pathogenic cells from extracellular polymeric substances (EPS) of a biofilm in a subject comprising administering a therapeutically effective amount of an aqueous hydrogel formulation selected from a hydrogel formulation of any one of embodiments 1-145 to a subject in need thereof.

Embodiment 147. A method of causing a regression of destabilizing overgrowth of pathogenic cells and symbiotic enablers in harmful biofilms in a subject comprising administering a therapeutically effective amount of an aqueous hydrogel formulation selected from a hydrogel formulation of any one of embodiments 1-145 to a subject in need thereof.

Embodiment 148. A method of inhibiting and/or preventing the attachment and formation of new biofilms by dispersed cells in a subject comprising administering a therapeutically effective amount of an aqueous hydrogel formulation selected from a hydrogel formulation of any one of embodiments 1-145 to a subject in need thereof.

Embodiment 149. A method of preventing, reducing, and/or inhibiting a biofilm in or on a subject comprising administering a therapeutically effective amount of an aqueous hydrogel formulation selected from a hydrogel formulation of any one of embodiments 1-145 to a subject in need thereof.

Embodiment 150. A method of treating or preventing an oral infection in a subject comprising administering a therapeutically effective amount of an aqueous hydrogel formulation selected from a hydrogel formulation of any one of embodiments 1-145 to a subject in need thereof.

Embodiment 151. A method of treating or preventing periodontitis in a subject comprising administering a therapeutically effective amount of an aqueous hydrogel formulation selected from a hydrogel formulation of any one of embodiments 1-145 to a subject in need thereof.

Embodiment 152. A method of treating or preventing gingivitis in a subject comprising administering a therapeutically effective amount of an aqueous hydrogel formulation selected from a hydrogel formulation of any one of embodiments 1-145 to a subject in need thereof.

Embodiment 153. A method of treating or preventing dental carriers in a subject comprising administering a therapeutically effective amount of an aqueous hydrogel formulation selected from a hydrogel formulation of any one of embodiments 1-145 to a subject in need thereof.

Embodiment 154. A method of treating or preventing an infection in a wound comprising administering a therapeutically effective amount of an aqueous hydrogel formulation selected from a hydrogel formulation of any one of embodiments 1-145 to a subject in need thereof.

Embodiment 155. The method of any one of embodiments 146-154, wherein the subject is a human.

Embodiment 156. The method of any one of embodiments 146-154, wherein the subject is a mammal.

Embodiment 157. The method of any one of embodiments 146-154, wherein the subject is a dog.

Embodiment 158. The method of any one of embodiments 146-154, wherein the subject is a cat.

In alternative aspects, non-limiting embodiments of the invention include:

Embodiment B1. A quaternary ammonium silane aqueous hydrogel formulation comprising:
a) a quaternary ammonium silane compound selected from the group consisting of Formula A-P and Compounds I-LIV;
b) a non-ionic gelling agent;
c) a non-ionic adhesion agent;
d) a non-ionic wetting agent;
e) optionally a non-ionic osmotic agent; and,
f) an aqueous solution,
wherein Formula A-P are:

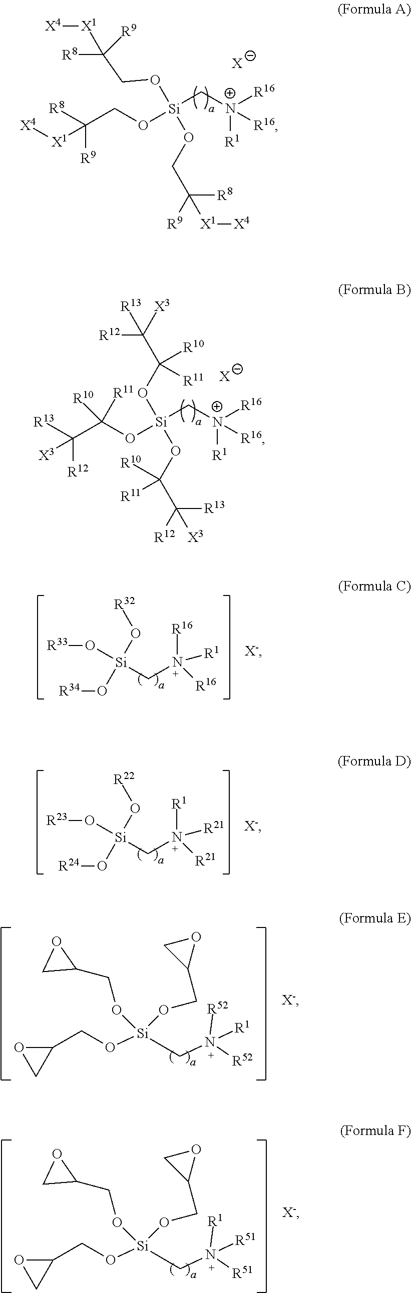

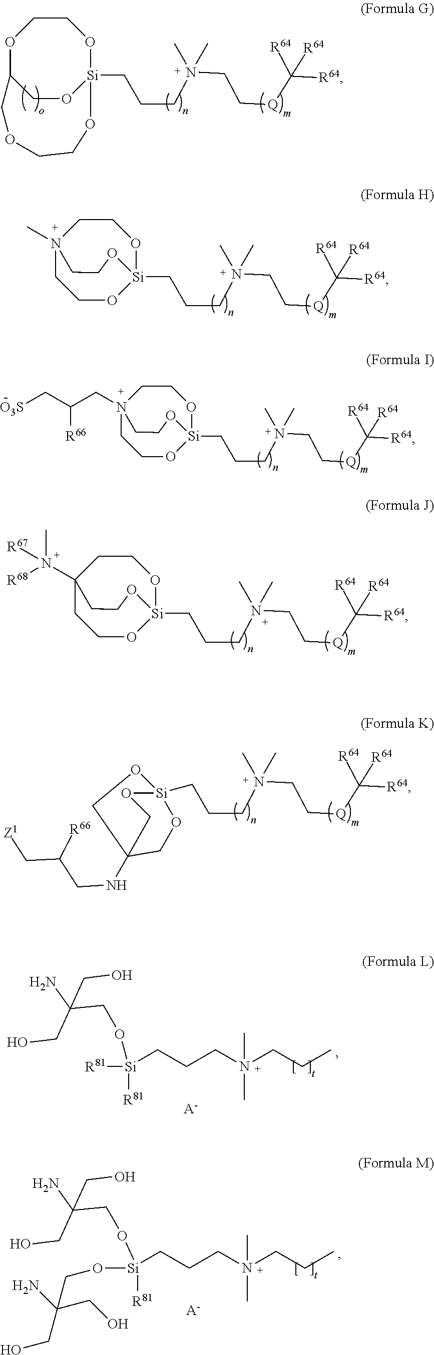

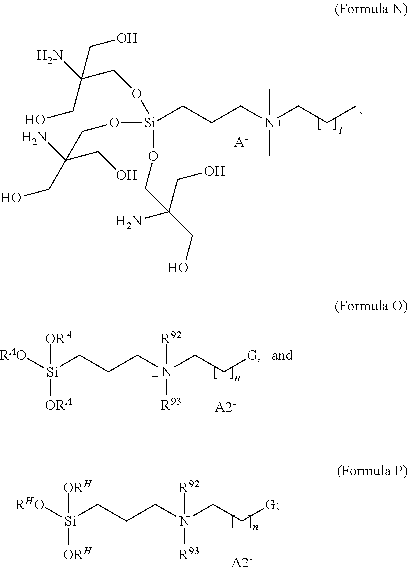

wherein:
a is 1, 2, 3, or 4;
m is 5, 6, 7, 8, 9, 10, 11, 12, 13, 14, 15, 16, or 17;
n is 0, 1, or 2;
o is 1 or 2;
each t is independently selected from the group consisting of 1, 2, 3, 4, 5, 6, 7, 8, 9, 10, 11, 12, 13, 14, 15, 16, 17, and 18;
R¹ is independently at each occurrence selected from the group consisting of C₆-C₂₂alkyl and C₆-C₂₂ alkanoyl;
R⁸ is H or CH₂OH;
R⁹ is H or C₁-C₈ alkyl;
X³ is OH or CH₂OR⁷;
R¹⁰, R¹¹, and R¹² are independently at each occurrence selected from the group consisting of hydrogen, hydroxyl, CH₂OR⁷, CON(R⁷)₂, COOR⁷, C(O)R⁷, C₁-C₈alkyl, C₁-C₈ hydroxyalkyl, and C₁-C₈ alkanoyl;
R¹³ is selected from the group consisting of hydroxyl, CH₂OR⁷, CON(R⁷)₂, COOR⁷, C(O)R⁷, C₁-C₈ alkyl, C₁-C₈ hydroxyalkyl, and C₁-C₈ alkanoyl;
R⁷ is independently at each occurrence selected from hydrogen, C₁-C₈ alkyl, C₁-C₈ hydroxyalkyl, and C₁-C₈ alkanoyl;
X¹ is NR¹⁷, CH₂, or C(O);
X⁴ is selected from the group consisting of hydroxyl, CH₂OR⁷, CON(R⁷)₂, COOR⁷,
C(O)R⁷, C₁-C₈ hydroxyalkyl, and C₁-C₈ alkanoyl;
R¹⁶ is C₁-C₄ alkyl;
R¹⁷ is hydrogen, C₁-C₈ hydroxyalkyl, or C₁-C₈ alkanoyl;
X is an anion or is absent if the quaternary amine is balanced with an internal anion;
R³², R³³, R³⁴, R²², R²³, and R²⁴ are independently at each occurrence selected from the group consisting of:

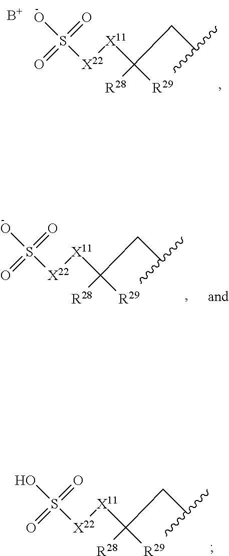

each R²¹ is independently selected from C₁-C₂₂alkyl and C₂-C₂₂ alkanoyl;
R²⁸ and R²⁹ are independently at each occurrence selected from hydrogen, halogen, hydroxyl, N (R⁷)₂, CH₂OR⁷, CON(R⁷)₂, COOR⁷, C(O)R⁷, C₁-C₈ alkyl, C₁-C₈ hydroxyalkyl, and C₁-C₈ alkanoyl;
X¹¹ is NR¹⁷, CH₂, CHOH, or C(O);
X²² is C₁-C₃alkyl or C₁-C₃hydroxyalkyl;
B⁺ is a cation;
R⁵² is independently at each occurrence selected from the group consisting of C₁-C₄ alkyl and C₁-C₄ haloalkyl;
each R⁵¹ is independently selected from C₁-C₂₂alkyl and C₂-C₂₂ alkanoyl; each of which R⁵¹ is optionally substituted with 1, 2, or 3, substituents independently selected from C₁-C₆ alkyl and halogen; and
Q is-CR⁶⁴R⁶⁴—;
R⁶⁴ is independently at each occurrence selected from hydrogen, C₁₋₃ alkyl, C₂₋₄ alkenyl, C₂₋₄ alkynyl, halogen, and C₁₋₃ haloalkyl;
R⁶⁶ is independently at each occurrence selected from hydrogen, hydroxy, and C₁-C₆ alkoxy;
R⁶⁷ and R⁶⁸ are independently at each occurrence selected from C₁-C₆ alkyl;

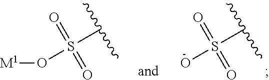

Z¹ is independently at each occurrence selected from
M¹ is hydrogen, sodium, potassium, cesium or lithium;
each R⁸¹ is independently selected from the group consisting of OH and alkoxy;
A⁻ is a non-halogen anion selected from the group consisting of

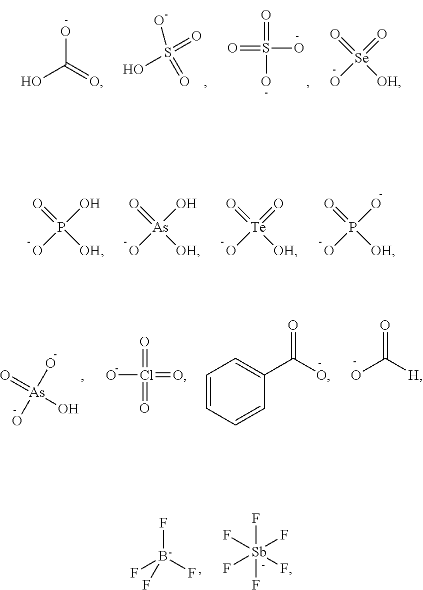

chlorite, perchlorate, hydroxide, formate, acetate, lactate, benzoate, nitrate, and salicylate anion;
R^{A} is selected from:

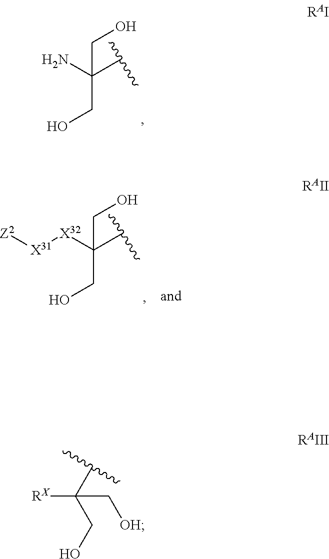

R^{H} is selected from:

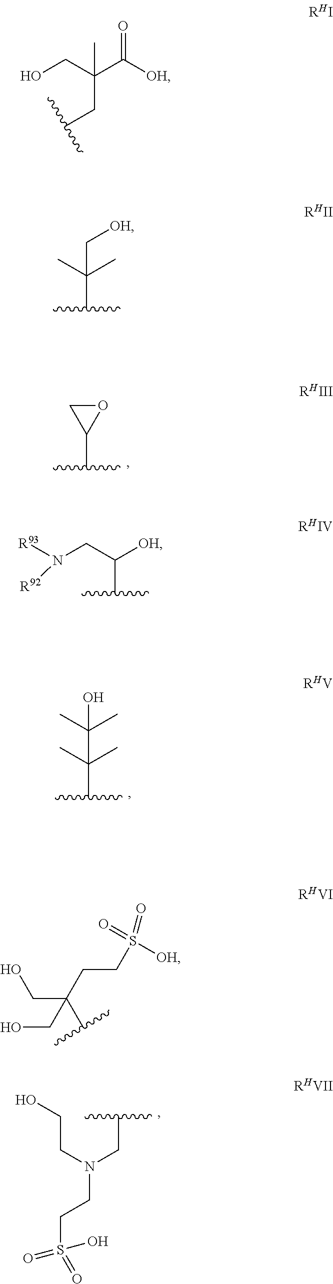

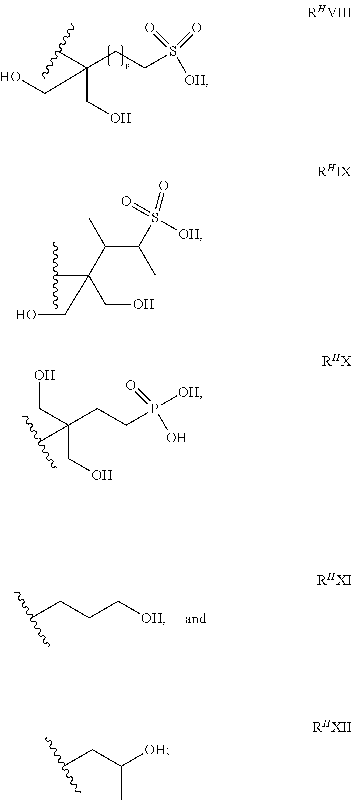

G is hydrogen, alkyl, aryl, cycloalkyl, heterocycle, heteroaryl, —CN, or CF₃;
each R⁹² is selected from hydrogen, C₁-C₆ alkyl, and —O;
v is selected from the group consisting of 1, 2, 3, 4, 5, 6, 7, 8, 9, 10, 11, 12, 13, 14, 15, 16, 17, and 18;
R⁹³ is selected from hydrogen, C₁-C₆ alkyl, and —O;

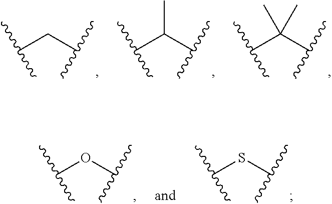

X³¹ is selected from the group consisting of a bond,
X³² is selected from the group consisting of a bond,

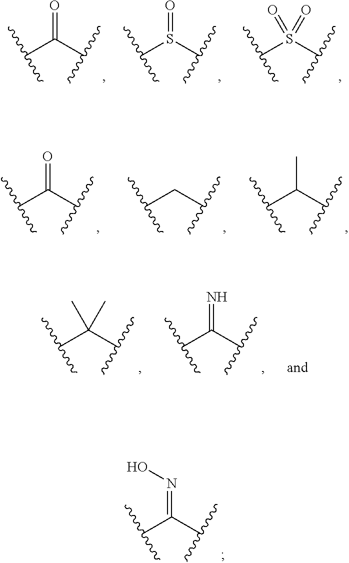

A2⁻ is an anion other than halogen or is absent if the compound of Formula O or Formula P is a charge balanced zwitterion, and wherein A2⁻ is selected from

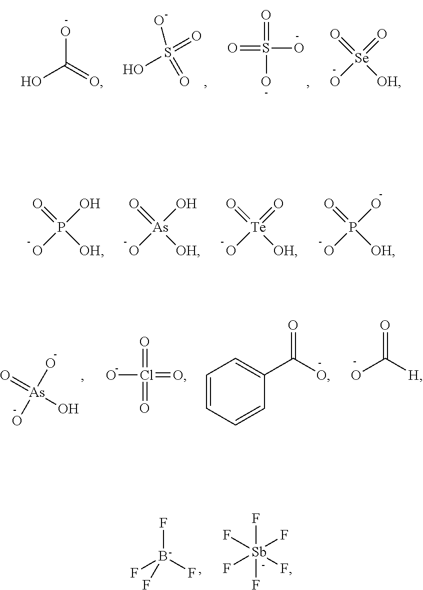

chlorite, perchlorate, hydroxide, formate, acetate, lactate, benzoate, nitrate, and salicylate anion;
R^{X} is selected from

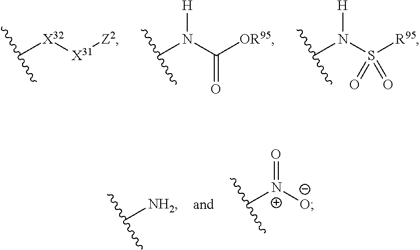

each R⁹⁵ is selected from hydrogen and C₁-C₆ alkyl;
Z² is selected from alkyl, —NO₂, —SO₃H, —SO₂H, —SO₃⁻, —SO₃M², —CO₂H, —CO₂⁻, —CO₂M², phosphate, OH, NH₂, and SH; and
M² is a metal cation selected from potassium cation, sodium cation, lithium cation, magnesium caution, and calcium cation; and
wherein Compounds I-LIV are:

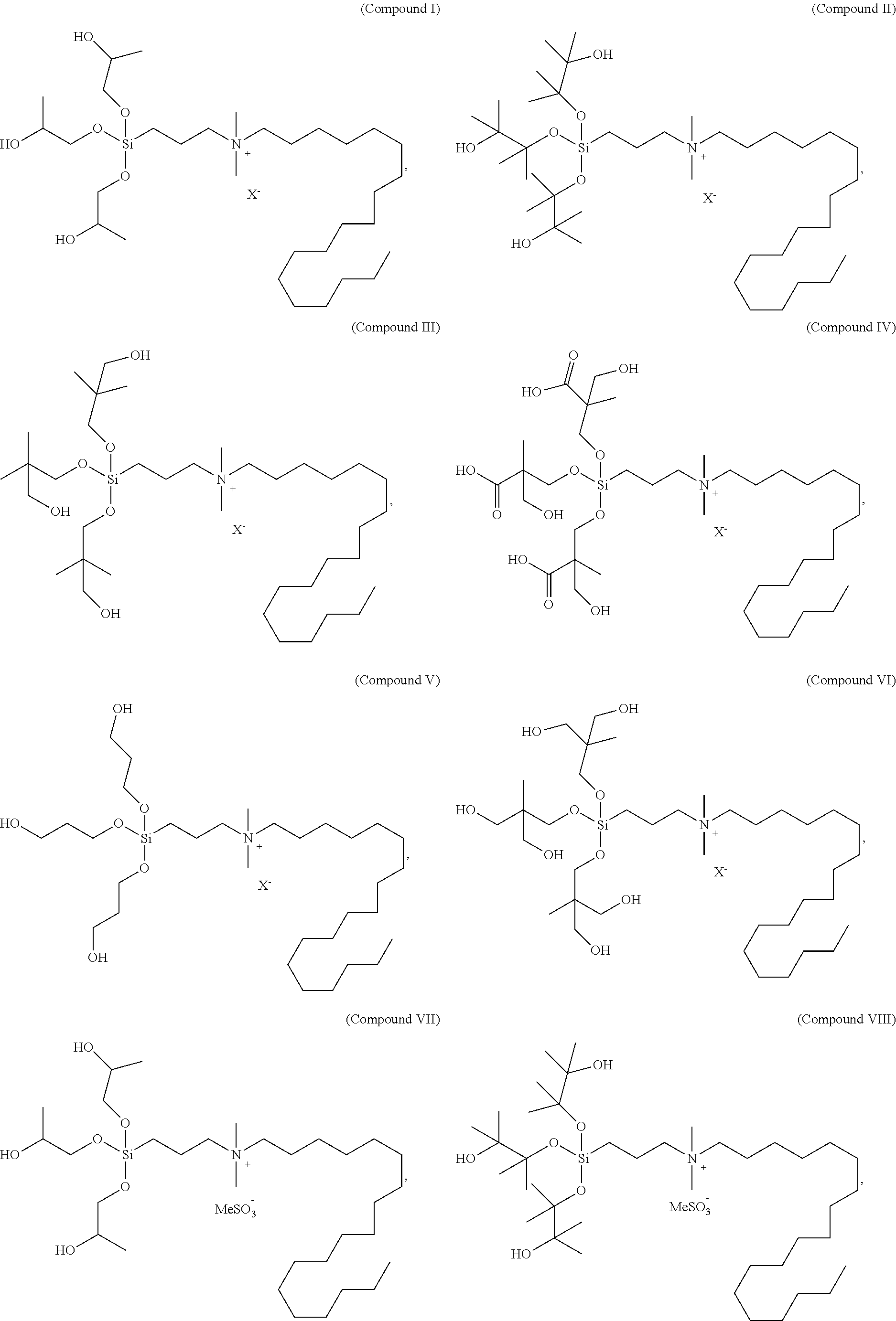

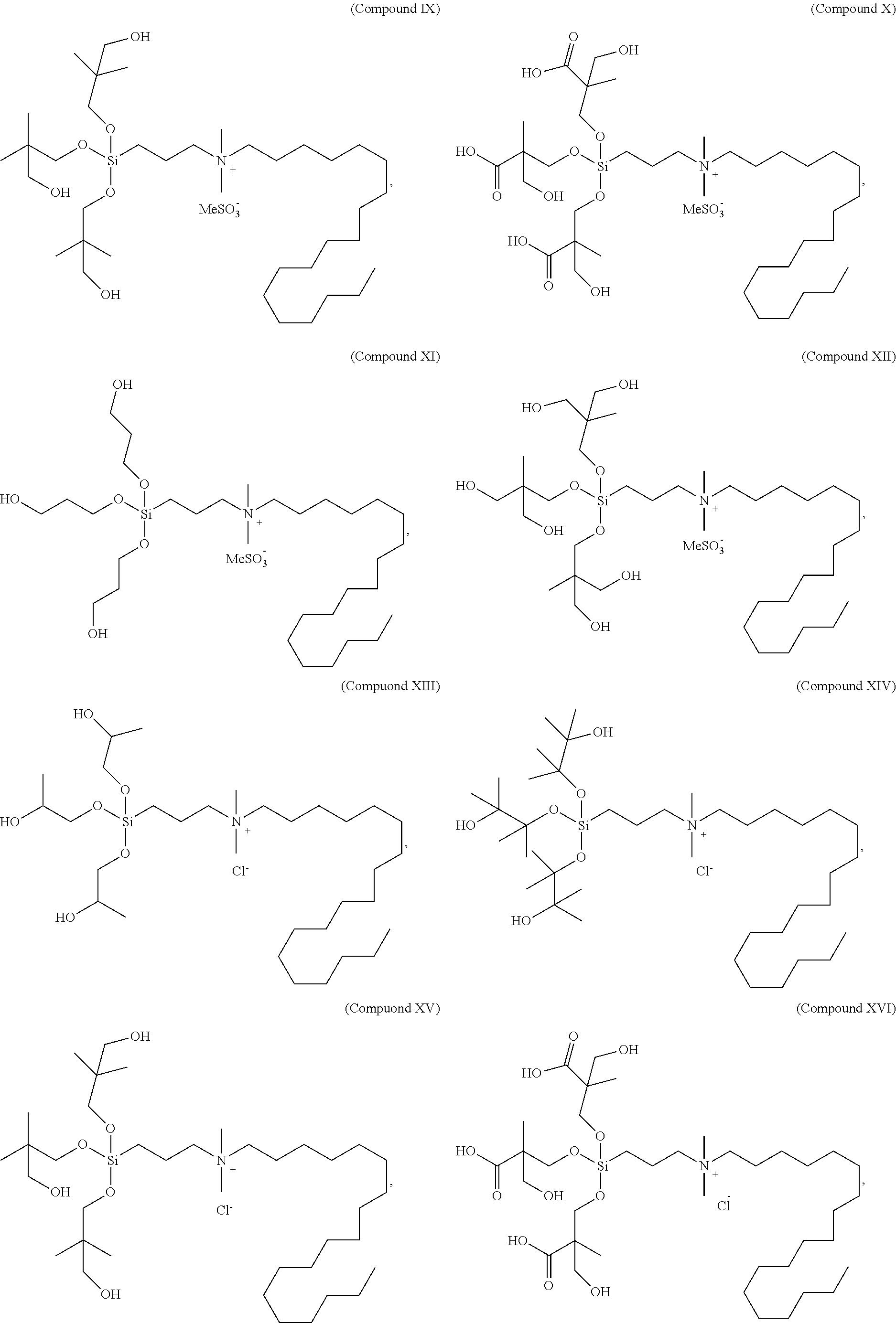

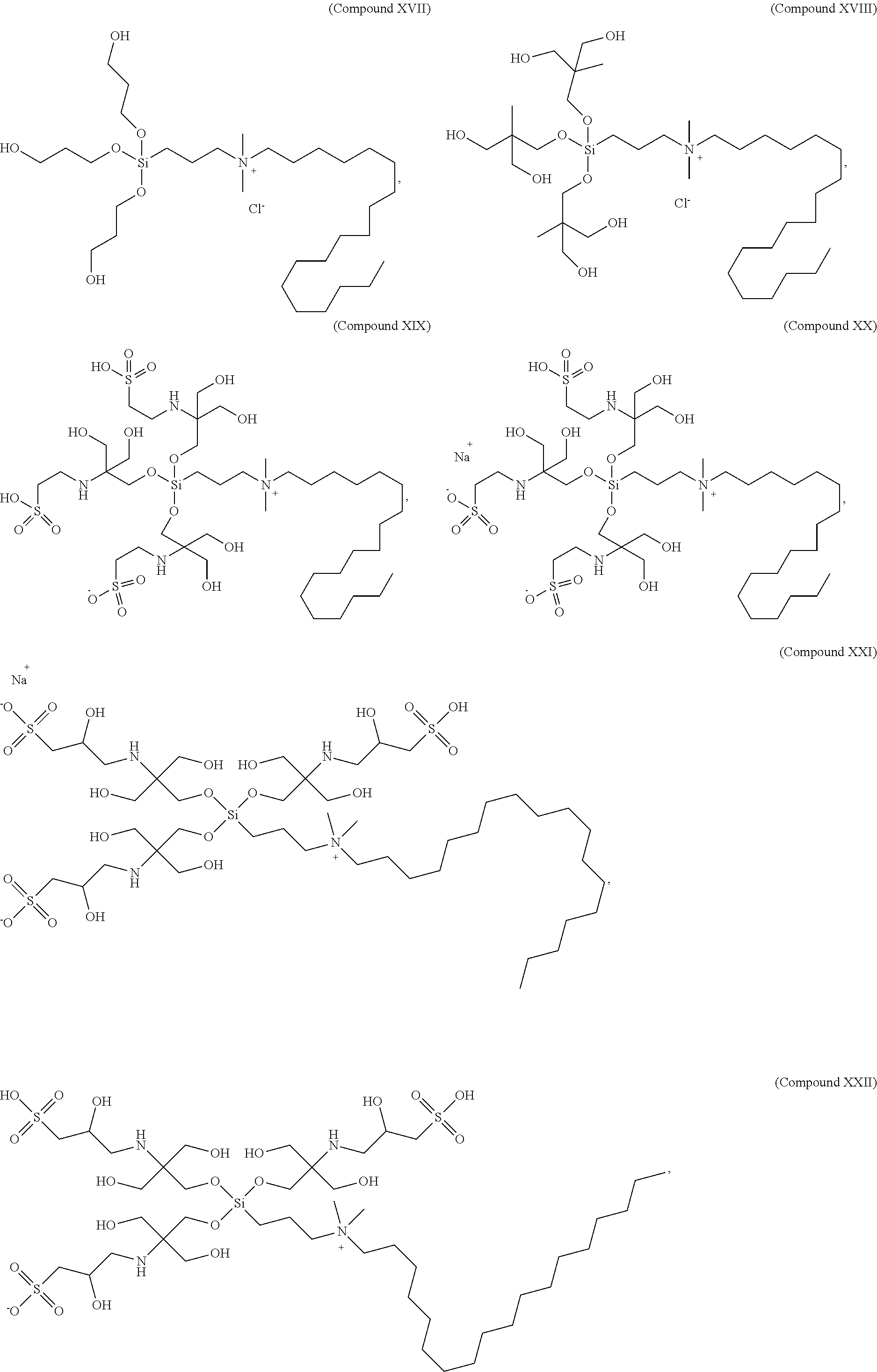

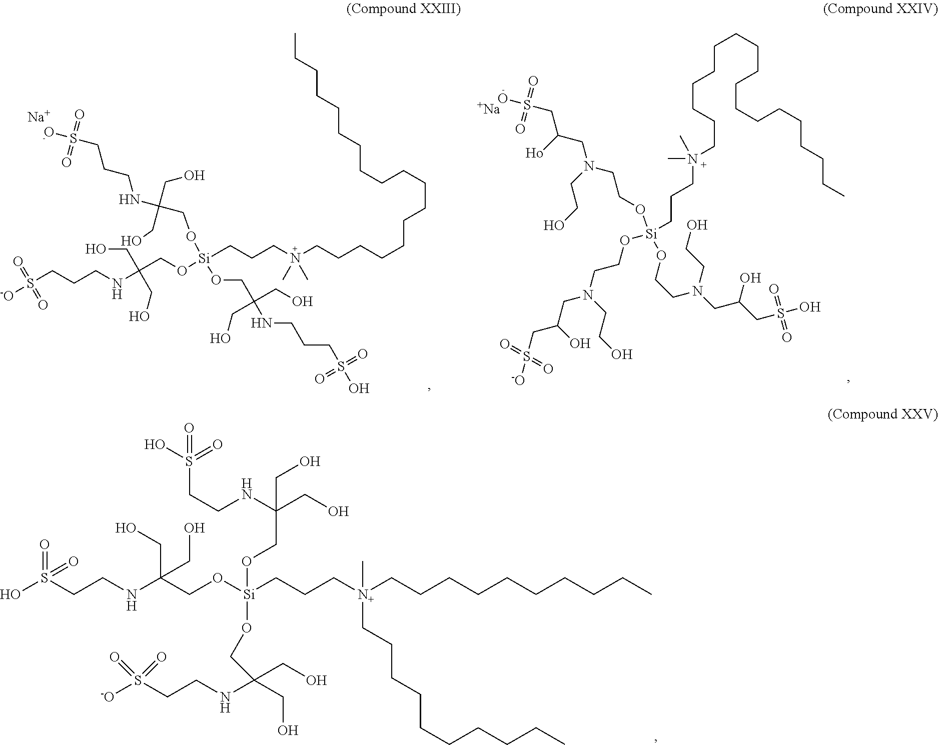

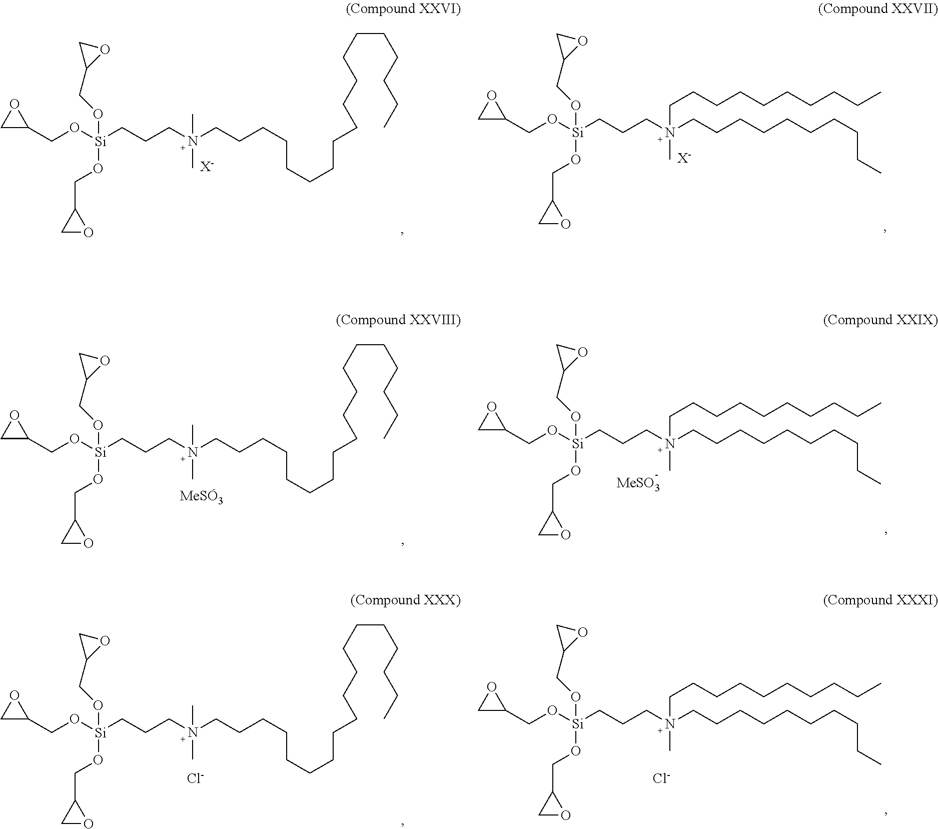

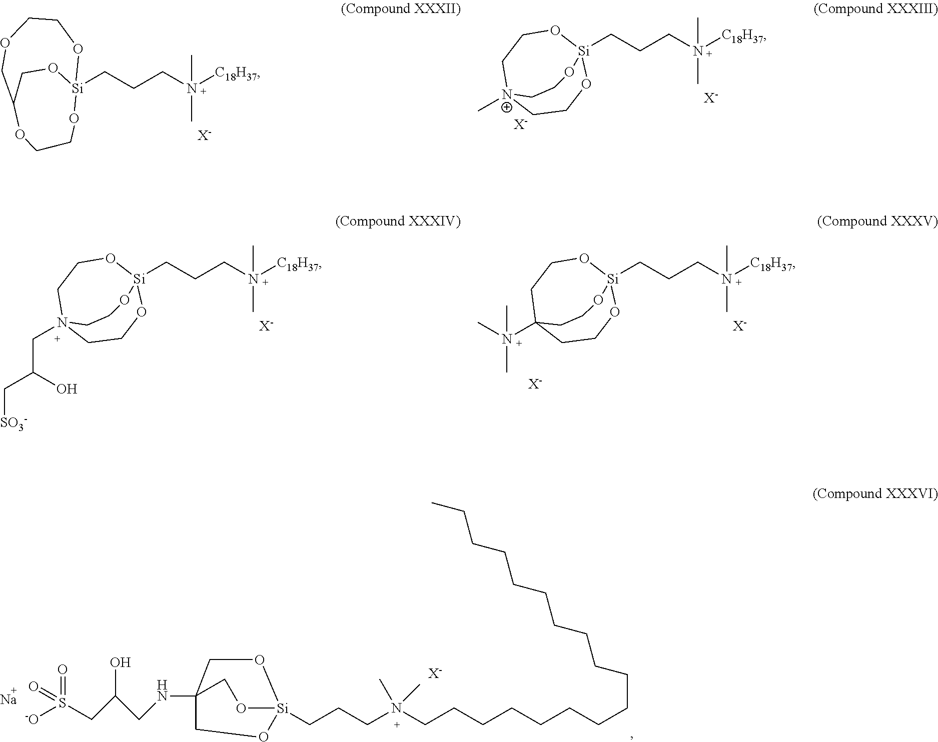

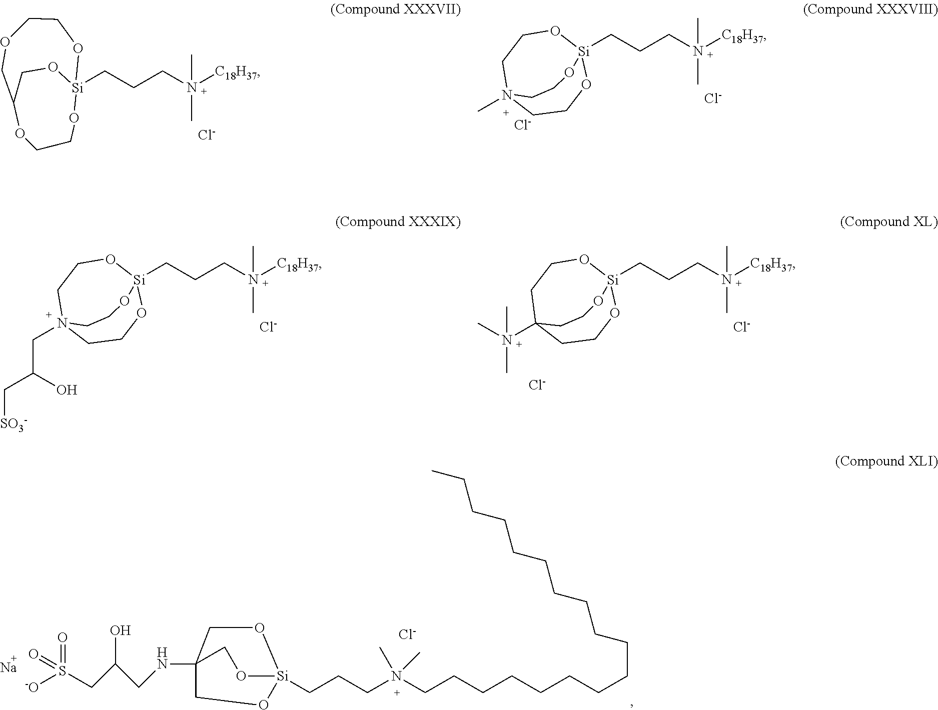

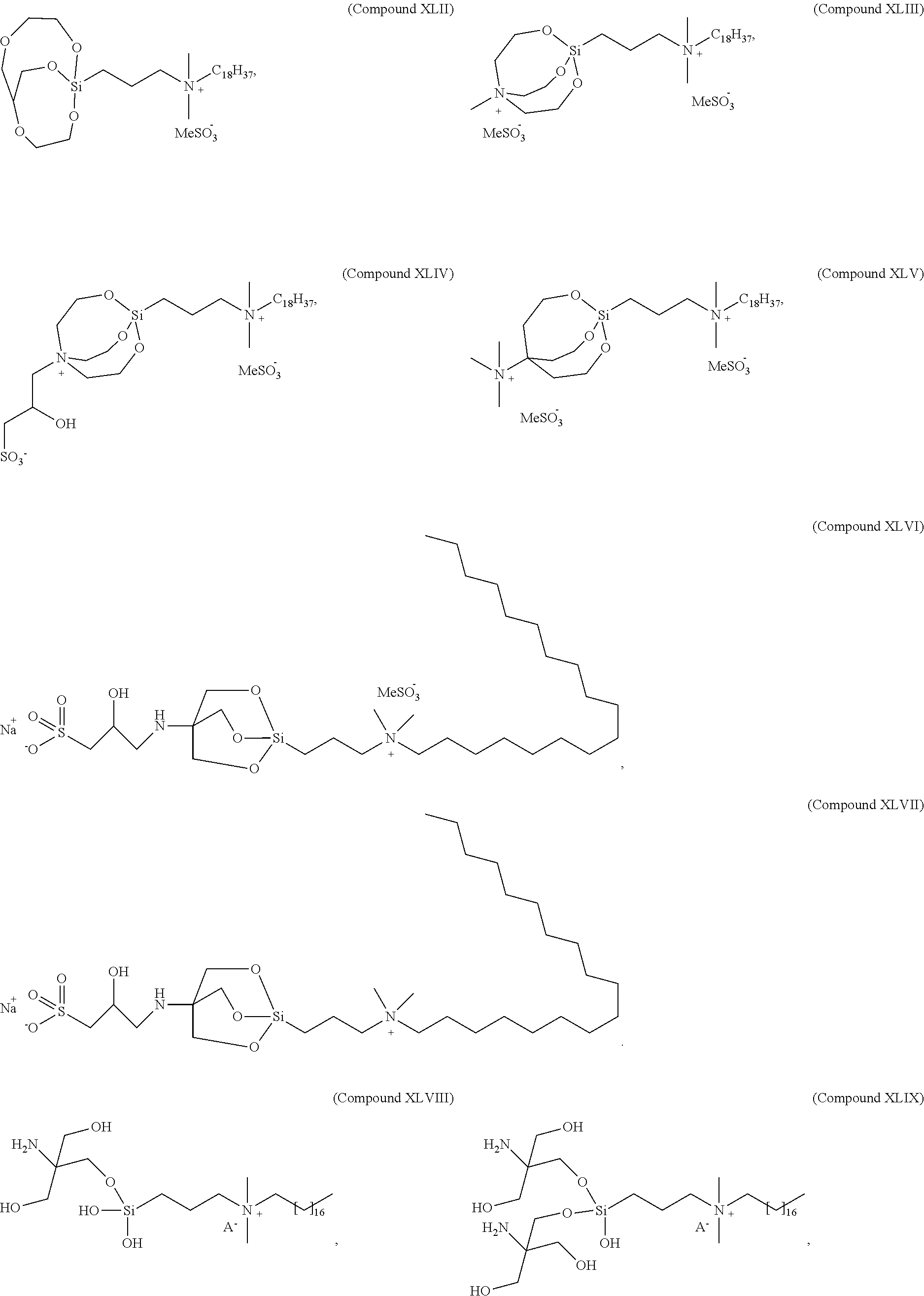

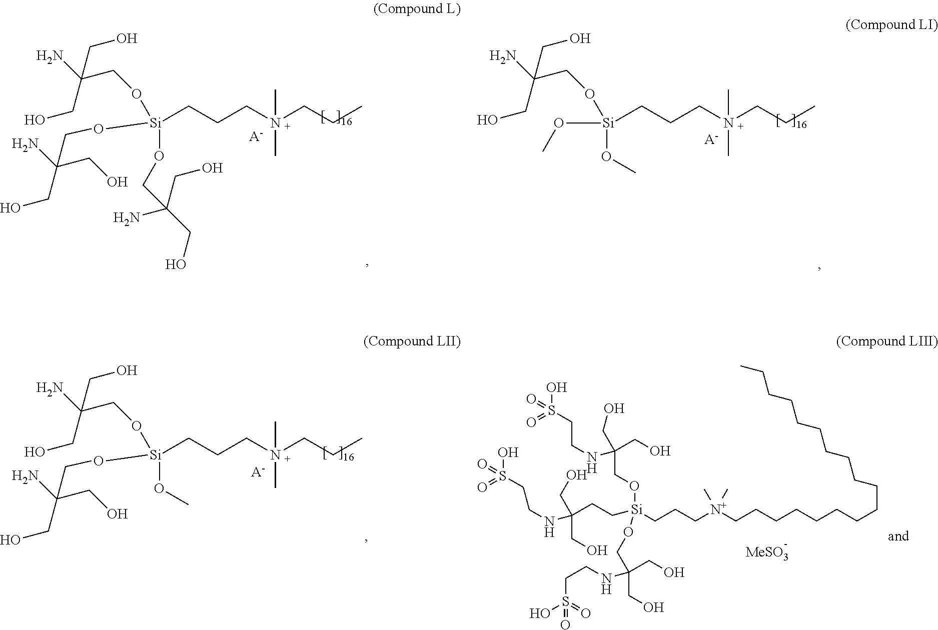

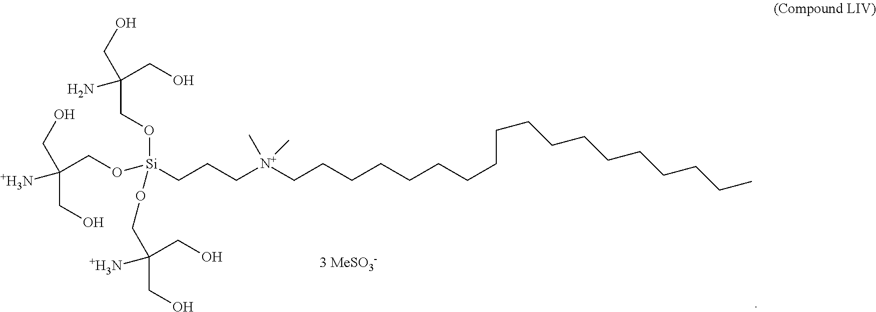 [SMILES: CCCCCCCCCCCCCCCCCC[N+](C)(C)CCC[Si](OCC(N)(CO)CO)(OCC([NH3+])(CO)CO)OCC([NH3+])(CO)CO]

Embodiment B2. The quaternary ammonium silane aqueous hydrogel formulation of embodiment 1 comprising:
a) between about 0.1% and 10% (weight/weight) (w/w) of a quaternary ammonium silane compound selected from the group consisting of Formula A-P and Compounds I-LIV;
b) between about 0.25% and about 3.0% (w/w) of the non-ionic gelling agent;
c) between about 0.025% and about 0.5% (w/w) of the non-ionic adhesion agent;
d) between about 0.5% and about 5.0% (w/w) of the non-ionic wetting agent;
e) optionally between about 0.5% and about 7.5% (w/w) of the non-ionic osmotic agent; and,
f) the aqueous solution.

Embodiment B3. The aqueous hydrogel formulation of embodiment B1 or B2 comprising:
a) a compound selected from the group consisting of Formula A-P and Compounds I-LIV, wherein the compound is present in the hydrogel formulation at a concentration that results in a trihydroxy-QAS cation present in the hydrogel at between about 0.1% and 5.0% (weight/weight) (w/w) when the compound is fully hydrolyzed, wherein the trihydroxy-QAS cation is of formula

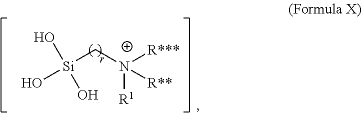

wherein
   r is 1, 2, 3, or 4;
   R¹ is C₆-C₂₂ alkyl or C₆-C₂₂ alkanoyl;
   R** and R*** are independently selected from the group consisting of:
   i. C₁-C₄ alkyl; and,
   ii. C₆-C₂₂alkyl or C₂-C₂₂alkanoyl;
b) between about 0.25% and about 3.0% (w/w) of a non-ionic gelling agent;
c) between about 0.025% and about 0.5% (w/w) of a non-ionic adhesion agent;
d) between about 0.5% and about 5.0% (w/w) of a non-ionic wetting agent; and,
e) optionally between about 0.5% and about 7.5% (w/w) of a non-ionic osmotic agent.

Embodiment B4. The aqueous hydrogel formulation of any one of embodiments B1-B3, wherein the trihydroxy-QAS cation is of structure:

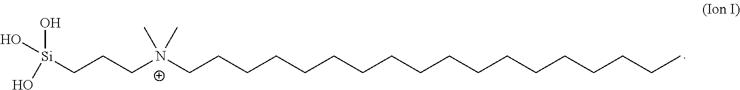 [SMILES: CCCCCCCCCCCCCCCCCC[N+](C)(C)CCC[Si](O)(O)O]

Embodiment B5. The aqueous hydrogel formulation of any one of embodiments B1-B4, wherein the non-ionic gelling agent is present in a concentration of about 2% (w/w).

Embodiment B6. The aqueous hydrogel formulation of any one of embodiments B1-B4, wherein the non-ionic gelling agent is present in a concentration of between about 0.5% (w/w) and 1.5% (w/w).

Embodiment B7. The aqueous hydrogel formulation of any one of embodiments B1-B4, wherein the non-ionic gelling agent is present in a concentration of between about 0.75% (w/w) and about 1.25% (w/w).

Embodiment B8. The aqueous hydrogel formulation of any one of embodiments B1-B4, wherein the non-ionic gelling agent is present in a concentration of about 1% (w/w).

Embodiment B9. The aqueous hydrogel formulation of any one of embodiments B1-B8, wherein the non-ionic gelling agent lacks a carboxylic acid group.

Embodiment B10. The aqueous hydrogel formulation of any one of embodiments B1-B9, wherein the non-ionic gelling agent is selected from the group consisting of hydoxypropyl methylcellulose (HPMC), hydroxyethyl cellulose, methyl cellulose, hydroxypropyl cellulose, polydextrose, palmitostearate, methacrylated inulin, and hydroxypropyl guar.

Embodiment B11. The aqueous hydrogel formulation of embodiment B10, wherein the non-ionic gelling agent is HPMC.

Embodiment B12. The aqueous hydrogel formulation of embodiment B11, wherein the HPMC has a viscosity of between about 2600 and 5600 cP at 2% in water at 20° C.

Embodiment B13. The aqueous hydrogel formulation of any one of embodiments B1-B12, wherein the non-ionic adhesion agent is present in the amount of between about 0.025% (w/w) and about 0.5% (w/w).

Embodiment B14. The aqueous hydrogel formulation of any one of embodiments B1-B13, wherein the non-ionic adhesion agent is present in the amount of between about 0.05% (w/w) and about 0.25% (w/w).

Embodiment B15. The aqueous hydrogel formulation of embodiment B14, wherein the non-ionic adhesion agent is present in the amount of about 0.2% (w/w).

Embodiment B16. The aqueous hydrogel formulation of any one of embodiments B1-B13, wherein the non-ionic adhesion agent is present in the amount of between about 0.075% (w/w) and about 0.15% (w/w).

Embodiment B17. The aqueous hydrogel formulation of embodiment B16, wherein the non-ionic adhesion agent is present in the amount of about 0.1% (w/w).

Embodiment B18. The aqueous hydrogel formulation of any one of embodiments B1-B17, wherein the non-ionic adhesion agent is selected from the group consisting of a poloxamer, polyoxypropylene, polyoxyethylene, and copolymers of polyoxypropylene and polyoxyethylene.

Embodiment B19. The aqueous hydrogel formulation of embodiment B18, wherein the non-ionic adhesion agent is a poloxamer.

Embodiment B20. The aqueous hydrogel formulation of embodiment B19, wherein the non-ionic adhesion agent is poloxamer 407.

Embodiment B21. The hydrogel formulation of any one of embodiments B1-B20, wherein the non-ionic wetting agent is present in a concentration of between about 1.0% (w/w) and 3.0% (w/w).

Embodiment B22. The aqueous hydrogel formulation of any one of embodiments B1-B21, wherein the non-ionic wetting agent is present in a concentration of between about 1.5% and about 2.5% (w/w).

Embodiment B23. The aqueous hydrogel formulation of embodiment B22, wherein the non-ionic wetting agent is present in a concentration of about 2.0% (w/w).

Embodiment B24. The aqueous hydrogel formulation of any one of embodiments B1-B23, wherein the non-ionic wetting agent lacks a carboxylic acid group.

Embodiment B25. The aqueous hydrogel formulation of any one of embodiments B1-B24, wherein the non-ionic wetting agent is selected from the group consisting of propylene glycol, polypropylene glycol, glycerin, glycerol, glycerol-propylene oxide copolymer, ethylene glycol, polyvinyl alcohol, and combinations thereof.

Embodiment B26. The aqueous hydrogel formulation of embodiment B25, wherein the non-ionic wetting agent is propylene glycol.

Embodiment B27. The aqueous hydrogel formulation of embodiment B25, wherein the non-ionic wetting agent is glycerine.

Embodiment B28. The aqueous hydrogel formulation of any one of embodiments B1-B27, wherein the hydrogel formulation further comprises a non-ionic osmotic agent.

Embodiment B29. The aqueous hydrogel formulation of any one of embodiments B1-B28, wherein the non-ionic osmotic agent is present in the hydrogel at a concentration of about 5.0% (w/w).

Embodiment B30. The aqueous hydrogel formulation of any one of embodiments B1-B28, wherein the non-ionic osmotic agent is present in the hydrogel at a concentration of between about 1.0% (w/w) and about 3.0% (w/w).

Embodiment B31. The aqueous hydrogel formulation of any one of embodiments B1-B28, wherein the non-ionic osmotic agent is present in the hydrogel at a concentration of between about 1.0% (w/w) and about 3.0% (w/w).

Embodiment B32. The aqueous hydrogel formulation of embodiment B31, wherein the non-ionic osmotic agent is present in the hydrogel at a concentration of about 2.0% (w/w).

Embodiment B33. The aqueous hydrogel formulation of any one of embodiments B1-B32, wherein the non-ionic osmotic agent is selected from the group consisting of polyethylene glycol (PEG), sorbitol, lactose, fructose, dextrose, lactulose, sucrose, xylitol, mannitol, and mixtures thereof.

Embodiment B34. The aqueous hydrogel formulation of any one of embodiments B1-B33, wherein the non-ionic osmotic agent is PEG.

Embodiment B35. The aqueous hydrogel formulation of embodiment B34, wherein the PEG has an average molecular weight (Mn) of between about 200 and about 100,000.

Embodiment B36. The aqueous hydrogel formulation of embodiment B35, wherein the PEG has an average Mn of 6000.

Embodiment B37. The aqueous hydrogel formulation of any one of embodiments B1-B33, wherein the non-ionic osmotic agent is sorbitol.

Embodiment B38. The aqueous hydrogel formulation of any one of embodiments B1-B37, wherein the aqueous solution is selected from the group of water, saline, or a phosphate buffered saline.

Embodiment B39. The aqueous hydrogel formulation of embodiment B38, wherein the aqueous solution is water.

Embodiment B40. The aqueous hydrogel formulation of any one of embodiments B1-B38, wherein the hydrogel comprises between about 0.25% and about 3% (w/w) trihydroxy-QAS cation.

Embodiment B41. The aqueous hydrogel formulation of any one of embodiments B1-B38, wherein the hydrogel comprises between about 0.25% and 1.0% (w/w) trihydroxy-QAS cation.

Embodiment B42. The aqueous hydrogel formulation of any one of embodiments B1-B38, wherein the hydrogel comprises between about 0.5% and about 0.75% (w/w) trihydroxy-QAS cation.

Embodiment B43. The aqueous hydrogel formulation of any one of embodiments B1-B38, wherein the hydrogel comprises about 0.5% (w/w) trihydroxy-QAS cation.

Embodiment B44. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 2.0% (w/w) of HPMC;
c. about 0.1% (w/w) of poloxamer 407;
d. about 2.0% (w/w) of propylene glycol;
e. about 2.0% (w/w) of PEG; and,
f. optionally one or more additional agents.

Embodiment B45. The aqueous formulation of embodiment B44, wherein the PEG has a Mn of 6000.

Embodiment B46. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 1.0% (w/w) of HPMC;
c. about 0.1% (w/w) of poloxamer 407;
d. about 2.0% (w/w) of propylene glycol;
e. about 2.0% (w/w) of PEG; and,
f. optionally one or more additional agents.

Embodiment B47. The aqueous formulation of embodiment B46, wherein the PEG has a Mn of 6000.

Embodiment B48. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 2.0% (w/w) of HPMC;
c. about 0.2% (w/w) of poloxamer 407;
d. about 2.0% (w/w) of glycerin;
e. about 5.0% (w/w) of a sorbitol; and,
f. optionally one or more additional agents.

Embodiment B49. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 1.0% (w/w) of HPMC;
c. about 0.2% (w/w) of poloxamer 407;
d. about 2.0% (w/w) of glycerin;
e. about 5.0% (w/w) of a sorbitol; and,
f. optionally one or more additional agents.

Embodiment B50. An aqueous hydrogel formulation comprising:
a. about 0.5% (w/w) of a trihydroxy-QAS cation of Formula X that is derived from the hydrolysis of a compound in the hydrogel selected from the group consisting of Formula A-P and Compounds I-LIV, or a combination thereof;
b. about 2.0% (w/w) of HPMC;
c. about 0.2% (w/w) of poloxamer 407;
d. about 2.0% (w/w) of glycerin; and,
e. optionally one or more additional agents.

Embodiment B51. An aqueous hydrogel formulation comprising:
a. between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b. between about 1.0% and about 2.2% (w/w) of a non-ionic gelling agent;
c. between about 0.15% to and about 0.25% (w/w) of a non-ionic adhesion agent;
d. between about 1.7% and about 2.3% (w/w) of a non-ionic wetting agent;
e. between about 4.0% and about 5.5% (w/w) of a non-ionic osmotic agent; and
f. optionally, between about 0.05% and about 0.4% (w/w) of an additional agent.

Embodiment B52. An aqueous hydrogel formulation comprising:
a. between about 0.3% and about 0.8% (w/w) of a trihydroxy-QAS cation of Formula X that is derived from the complete hydrolysis of a compound in the hydrogel selected from the group consisting of Formula A-P and Compounds I-LIV, or a combination thereof;
b. between about 1.0% and about 2.2% (w/w) of a non-ionic gelling agent;
c. between about 0.15% to and about 0.25% (w/w) of a non-ionic adhesion agent;
d. between about 1.7% and about 2.3% (w/w) of a non-ionic wetting agent; and
e. optionally, between about 0.05% and about 0.4% (w/w) of an additional agent.

Embodiment B53. The aqueous hydrogel formulation of embodiment B51 comprising: Embodiment B53.
a. between about 0.4% and about 0.6% (w/w) of a trihydroxy-QAS cation of Formula X that is derived from the complete hydrolysis of Compound XIX;
b. between about 1.8% and about 2.2% (w/w) of the non-ionic gelling agent, wherein the non-ionic gelling agent is HPMC;
c. between about 0.15% to and about 0.25% (w/w) of the non-ionic adhesion agent, wherein the non-ionic adhesion agent is Poloxamer 407;
d. between about 1.7% and about 2.3% (w/w) of the non-ionic wetting agent, wherein the non-ionic wetting agent is glycerin;
e. between about 4.5% and about 5.5% (w/w) of the non-ionic osmotic agent, wherein the non-ionic osmotic agent is sorbitol; and
f. between about 0.05% and about 0.2% (w/w) of the additional agent, wherein the additional agent is zinc gluconate.

Embodiment B54. The aqueous hydrogel formulation of embodiment B52 comprising:
a. between about 0.4% and about 0.6% (w/w) of a trihydroxy-QAS cation of Formula X that is derived from the complete hydrolysis of Compound XIX;
b. between about 1.8% and about 2.2% (w/w) of the non-ionic gelling agent, wherein the non-ionic gelling agent is HPMC;
c. between about 0.15% to and about 0.25% (w/w) of the non-ionic adhesion agent, wherein the non-ionic adhesion agent is Poloxamer 407;
d. between about 1.7% and about 2.3% (w/w) of the non-ionic wetting agent, wherein the non-ionic wetting agent is glycerin; and
e. between about 0.05% and about 0.2% (w/w) of the additional agent, wherein the additional agent is zinc gluconate.

Embodiment B55. The aqueous hydrogel formulation of any one of embodiments B46-B54, wherein the HPMC has a viscosity of between about 2600 and 5600 cP at 2% in water at 20° C.

Embodiment B56. The aqueous hydrogel formulation of any one of embodiments B1-B55, wherein the hydrogel formulation has a pH between about 5 and about 8.

Embodiment B57. The aqueous hydrogel formulation of any one of embodiments B1-B55, wherein the hydrogel formulation has a pH between about 5.5 and about 6.5.

Embodiment B58. The aqueous hydrogel formulation of any one of embodiments B1-B55, wherein the hydrogel formulation has a a pH of about 6.

Embodiment B59. The aqueous hydrogel formulation of any one of embodiments B1-B58, wherein the hydrogel formulation further comprises a pH adjusting agent.

Embodiment B60. The aqueous hydrogel formulation of embodiment B59, wherein the pH adjusting agent is methanesulfonic acid.

Embodiment B61. The aqueous hydrogel formulation of any one of embodiments B1-B60, wherein the hydrogel formulation further comprises a buffer.

Embodiment B62. The aqueous hydrogel formulation of any one of embodiments 1-61, wherein the hydrogel formulation has a viscosity of between about 1000 and 2000 cP.

Embodiment B63. The aqueous hydrogel formulation of any one of embodiments B1-B61, wherein the hydrogel formulation has a viscosity of between about 1250 and 1600 cP.

Embodiment B64. The aqueous hydrogel formulation of any one of embodiments B1-B61, wherein the hydrogel formulation has a viscosity of about 1600 cP.

Embodiment B65. The aqueous hydrogel formulation of any one of embodiments B1-B61, wherein the hydrogel formulation has a viscosity of about 1250 cP.

Embodiment B66. The aqueous hydrogel formulation of any one of embodiments B1-B65, wherein the hydrogel formulation further comprises an agent that disrupts and/or destabilizes the extracellular polymeric substances (EPS) that make up a biofilm (an “EPS disrupter”).

Embodiment B67. The aqueous hydrogel formulation of embodiment B66, wherein the EPS disrupter is selected from the group consisting of N-acetylcysteine, guaifenesin, erdosteinum, norspermidine, and a combination thereof.

Embodiment B68. The aqueous hydrogel formulation of embodiment B66, wherein the EPS disrupter is a D-amino acid, or a pharmaceutically acceptable salt, ester, or derivative thereof.

Embodiment B69. The aqueous hydrogel formulation of embodiment B68, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

Embodiment B70. The aqueous hydrogel formulation of embodiment B66, wherein the EPS Embodiment B70. disrupter is a proteolytic enzyme.

Embodiment B71. The aqueous hydrogel formulation of embodiment B70, wherein the proteolytic enzyme is selected from the group consisting of collagenase, cellulase, keratinase, papain, bromelain, trypsin, thermolysin, and combinations thereof.

Embodiment B72. The aqueous hydrogel formulation of any one of embodiments B1-B71, wherein the hydrogel formulation further comprises a plaque controlling agent.

Embodiment B73. The aqueous hydrogel formulation of embodiment B72, wherein the plaque controlling agent is selected from the group consisting of zinc gluconate, chlorhexidine digluconate, arginine, alexidine, octenidine HCl, iodine, iodophores, fluorides, calcium gluconate, bromelain, chitosan, fluorohydroxyapatite, hydroxyapatite, hypothiocyanite, Mg—Sr-carbonate hydroxyapatite conjugated with chitosan, nicotinyl alcohol HF, olaflur, papain, potassium fluoride, sodium fluoride, sodium monofluorophosphate, and stannous fluoride.

Embodiment B74. The aqueous hydrogel formulation of embodiment B73, wherein the plaque controlling agent is zinc gluconate.

Embodiment B75. The aqueous hydrogel formulation of any one of embodiments B1-B74, wherein the compound is a compound of Formula A.

Embodiment B76. The aqueous hydrogel formulation of any one of embodiments B1-B74, wherein the compound is a compound of Formula A-1.

Embodiment B77. The aqueous hydrogel formulation of any one of embodiments B1-B74, wherein the compound is a compound of Formula A-2.

Embodiment B78. The aqueous hydrogel formulation of any one of embodiments B1-B74, wherein the compound is a compound of Formula A-3.

Embodiment B79. The aqueous hydrogel formulation of any one of embodiments B1-B74, wherein the compound is a compound of Formula B.

Embodiment B80. The aqueous hydrogel formulation of any one of embodiments B1-B74, wherein the compound is a compound of Formula B-1.

Embodiment B81. The aqueous hydrogel formulation of any one of embodiments B1-B74, wherein the compound is a compound of Formula B-2.

Embodiment B82. The aqueous hydrogel formulation of any one of embodiments B1-B74, wherein the compound is a compound of Formula B-3.

Embodiment B83. The aqueous hydrogel formulation of any one of embodiments B1-B74, wherein the compound is Compound I.

Embodiment B84. The aqueous hydrogel formulation of any one of embodiments B1-B74, wherein the compound is Compound II.

Embodiment B85. The aqueous hydrogel formulation of any one of embodiments B1-B74, wherein the compound is Compound III.

Embodiment B86. The aqueous hydrogel formulation of any one of embodiments B1-B74, wherein the compound is Compound IV.

Embodiment B87. The aqueous hydrogel formulation of any one of embodiments B1-B74, wherein the compound is Compound V.

Embodiment B88. The aqueous hydrogel formulation of any one of embodiments B1-B74, wherein the compound is Compound VI.

Embodiment B89. The aqueous hydrogel formulation of any one of embodiments B1-B74, wherein the compound is Compound VII.

Embodiment B90. The aqueous hydrogel formulation of any one of embodiments B1-B74, wherein the compound is Compound VIII.

Embodiment B91. The aqueous hydrogel formulation of any one of embodiments B1-B74, wherein the compound is Compound IX.

Embodiment B92. The aqueous hydrogel formulation of any one of embodiments B1-B74, wherein the compound is Compound X.

Embodiment B93. The aqueous hydrogel formulation of any one of embodiments B1-B74, wherein the compound is Compound XI.

Embodiment B94. The aqueous hydrogel formulation of any one of embodiments B1-B74, wherein the compound is Compound XII.

Embodiment B95. The aqueous hydrogel formulation of any one of embodiments B1-B74, wherein the compound is Compound XIII.

Embodiment B96. The aqueous hydrogel formulation of any one of embodiments B1-B74, wherein the compound is Compound XIV.

Embodiment B97. The aqueous hydrogel formulation of any one of embodiments B1-B74, wherein the compound is Compound XV.

Embodiment B98. The aqueous hydrogel formulation of any one of embodiments B1-B74, wherein the compound is Compound XVI.

Embodiment B99. The aqueous hydrogel formulation of any one of embodiments B1-B74, wherein the compound is Compound XVII.

Embodiment B100. The aqueous hydrogel formulation of any one of embodiments B1-B74, wherein the compound is Compound XIII.

Embodiment B101. The aqueous hydrogel formulation of any one of embodiments B1-B74, wherein the compound is a compound of Formula C.

Embodiment B102. The aqueous hydrogel formulation of any one of embodiments B1-B74, wherein the compound is a compound of Formula D.

Embodiment B103. The aqueous hydrogel formulation of any one of embodiments B1-B74, wherein the compound is Compound XIX.

Embodiment B104. The aqueous hydrogel formulation of any one of embodiments B1-B74, wherein the compound is Compound XX.

Embodiment B105. The aqueous hydrogel formulation of any one of embodiments B1-B74, wherein the compound is Compound XXI.

Embodiment B106. The aqueous hydrogel formulation of any one of embodiments B1-B74, wherein the compound is Compound XXII.

Embodiment B107. The aqueous hydrogel formulation of any one of embodiments B1-B74, wherein the compound is Compound XXIII.

Embodiment B108. The aqueous hydrogel formulation of any one of embodiments B1-B74, wherein the compound is Compound XIV.

Embodiment B109. The aqueous hydrogel formulation of any one of embodiments B1-B74, wherein the compound is Compound XXV.

Embodiment B110. The aqueous hydrogel formulation of any one of embodiments B1-B74, wherein the compound is a compound of Formula E.

Embodiment B111. The aqueous hydrogel formulation of any one of embodiments B1-B74, wherein the compound is a compound of Formula F.

Embodiment B112. The aqueous hydrogel formulation of any one of embodiments B1-B74, wherein the compound is a compound of Compound XXVI.

Embodiment B113. The aqueous hydrogel formulation of any one of embodiments B1-B74, wherein the compound is a compound of Compound XXVII.

Embodiment B114. The aqueous hydrogel formulation of any one of embodiments B1-B74, wherein the compound is a compound of Compound XXVIII.

Embodiment B115. The aqueous hydrogel formulation of any one of embodiments B1-B74, wherein the compound is a compound of Compound XXIX.

Embodiment B116. The aqueous hydrogel formulation of any one of embodiments B1-B74, wherein the compound is a compound of Compound XXX.

Embodiment B117. The aqueous hydrogel formulation of any one of embodiments B1-B74, wherein the compound is a compound of Compound XXXI.

Embodiment B118. The aqueous hydrogel formulation of any one of embodiments B1-B74, wherein the compound is a compound of Formula G.

Embodiment B119. The aqueous hydrogel formulation of any one of embodiments B1-B74, wherein the compound is a compound of Formula H.

Embodiment B120. The aqueous hydrogel formulation of any one of embodiments B1-B74, wherein the compound is a compound of Formula I.

Embodiment B121. The aqueous hydrogel formulation of any one of embodiments B1-B74, wherein the compound is a compound of Formula J.

Embodiment B122. The aqueous hydrogel formulation of any one of embodiments B1-B74, wherein the compound is a compound of Formula K.

Embodiment B123. The aqueous hydrogel formulation of any one of embodiments B1-B74, wherein the compound is a compound of Compound XXXII.

Embodiment B124. The aqueous hydrogel formulation of any one of embodiments B1-B74, wherein the compound is a compound of Compound XXXIII.

Embodiment B125. The aqueous hydrogel formulation of any one of embodiments B1-B74, wherein the compound is a compound of Compound XXXIV.

Embodiment B126. The aqueous hydrogel formulation of any one of embodiments B1-B74, wherein the compound is a compound of Compound XXXV.

Embodiment B127. The aqueous hydrogel formulation of any one of embodiments B1-B74, wherein the compound is a compound of Compound XXXVI.

Embodiment B128. The aqueous hydrogel formulation of any one of embodiments B1-B74, wherein the compound is a compound of Compound XXXVII.

Embodiment B129. The aqueous hydrogel formulation of any one of embodiments B1-B74, wherein the compound is a compound of Compound XXXVIII.

Embodiment B130. The aqueous hydrogel formulation of any one of embodiments B1-B74, wherein the compound is a compound of Compound XXXIX.

Embodiment B131. The aqueous hydrogel formulation of any one of embodiments B1-B74, wherein the compound is a compound of Compound XL.

Embodiment B132. The aqueous hydrogel formulation of any one of embodiments B1-B74, wherein the compound is a compound of Compound XLI.

Embodiment B133. The aqueous hydrogel formulation of any one of embodiments B1-B74, wherein the compound is a compound of Compound XLII.

Embodiment B134. The aqueous hydrogel formulation of any one of embodiments B1-B74, wherein the compound is a compound of Compound XLIII.

Embodiment B135. The aqueous hydrogel formulation of any one of embodiments B1-B74, wherein the compound is a compound of Compound XLIV.

Embodiment B136. The aqueous hydrogel formulation of any one of embodiments B1-B74, wherein the compound is a compound of Compound XLV.

Embodiment B137. The aqueous hydrogel formulation of any one of embodiments B1-B74, wherein the compound is a compound of Compound XLVI.

Embodiment B138. The aqueous hydrogel formulation of any one of embodiments B1-B74, wherein the compound is a compound of Compound XLVII.

Embodiment B139. The aqueous hydrogel formulation of any one of embodiments B1-B74, wherein the compound is a compound of Formula L.

Embodiment B140. The aqueous hydrogel formulation of any one of embodiments B1-B74, wherein the compound is a compound of Formula M.

Embodiment B141. The aqueous hydrogel formulation of any one of embodiments B1-B74, wherein the compound is a compound of Formula N.

Embodiment B142. The aqueous hydrogel formulation of any one of embodiments B1-B74, wherein the compound is Compound XL VIII.

Embodiment B143. The aqueous hydrogel formulation of any one of embodiments B1-B74, wherein the compound is Compound XLIX.

Embodiment B144. The aqueous hydrogel formulation of any one of embodiments B1-B74, wherein the compound is Compound L.

Embodiment B145. The aqueous hydrogel formulation of any one of embodiments B1-B74, wherein the compound is Compound LI.

Embodiment B146. The aqueous hydrogel formulation of any one of embodiments B1-B74, wherein the compound is Compound LII.

Embodiment B147. The aqueous hydrogel formulation of any one of embodiments B1-B74, wherein the compound is a compound of Formula O.

Embodiment B148. The aqueous hydrogel formulation of any one of embodiments B1-B74, wherein the compound is a compound of Formula P.

Embodiment B149. The aqueous hydrogel formulation of any one of embodiments B1-B74, wherein the compound is Compound LIII.

Embodiment B150. The aqueous hydrogel formulation of any one of embodiments B1-B74, wherein the compound is Compound LIV.

Embodiment B151. A method of inhibiting the detachment and/or dissemination of pathogenic cells from extracellular polymeric substances (EPS) of a biofilm in a subject comprising administering a therapeutically effective amount of an aqueous hydrogel formulation selected from a hydrogel formulation of any one of embodiments B1-B150 to a subject in need thereof.

Embodiment B152. A method of causing a regression of destabilizing overgrowth of pathogenic cells and symbiotic enablers in harmful biofilms in a subject comprising administering a therapeutically effective amount of an aqueous hydrogel formulation selected from a hydrogel formulation of any one of embodiments B1-B150 to a subject in need thereof.

Embodiment B153. A method of inhibiting and/or preventing the attachment and formation of new biofilms by dispersed cells in a subject comprising administering a therapeutically effective amount of an aqueous hydrogel formulation selected from a hydrogel formulation of any one of embodiments B1-B150 to a subject in need thereof.

Embodiment B154. A method of preventing, reducing, and/or inhibiting a biofilm in or on a subject comprising administering a therapeutically effective amount of an aqueous hydrogel formulation selected from a hydrogel formulation of any one of embodiments B1-B150 to a subject in need thereof.

Embodiment B155. A method of treating or preventing an oral infection in a subject comprising administering a therapeutically effective amount of an aqueous hydrogel formulation selected from a hydrogel formulation of any one of embodiments B1-B150 to a subject in need thereof.

Embodiment B156. The method of embodiment B155, wherein the oral infection is stomatitis. Embodiment B156.

Embodiment B157. The method of embodiment B155, wherein the oral infection is pharyngitis.

Embodiment B158. The method of embodiment B155, wherein the oral infection is gingivitis.

Embodiment B159. The method of embodiment B155, wherein the oral infection is periodontitis.

Embodiment B160. A method of treating or preventing periodontitis in a subject comprising administering a therapeutically effective amount of an aqueous hydrogel formulation selected from a hydrogel formulation of any one of embodiments B1-B150 to a subject in need thereof.

Embodiment B161. A method of treating or preventing gingivitis in a subject comprising administering a therapeutically effective amount of an aqueous hydrogel formulation selected from a hydrogel formulation of any one of embodiments B1-B150 to a subject in need thereof.

Embodiment B162. A method of treating or preventing dental carriers in a subject comprising administering a therapeutically effective amount of an aqueous hydrogel formulation selected from a hydrogel formulation of any one of embodiments B1-B150 to a subject in need thereof.

Embodiment B163. A method of treating or preventing an infection in a wound comprising administering a therapeutically effective amount of an aqueous hydrogel formulation selected from a hydrogel formulation of any one of embodiments B1-B150 to a subject in need thereof.

Embodiment B164. The method of any one of embodiments B151-B163, wherein the subject is a human.

Embodiment B165. The method of any one of embodiments B151-B163, wherein the subject is a mammal.

Embodiment B166. The method of any one of embodiments B151-B163, wherein the subject is a dog.

Embodiment B167. The method of any one of embodiments B151-B163, wherein the subject is a cat.

Embodiment B168. Use of an aqueous hydrogel formulation of any one of embodiments B1-B150 in the manufacture of a medicament for the treatment or prevention of an oral infection in a subject in need thereof.

Embodiment B169. Use of an aqueous hydrogel formulation of any one of embodiments B1-B150 for the treatment or prevention of an oral infection in a subject in need thereof.

Embodiment B170. The use of embodiment B168 or B169, wherein the oral infection is stomatitis.

Embodiment B171. The use of embodiment B168 or B169, wherein the oral infection is pharyngitis.

Embodiment B172. The use of embodiment B168 or B169, wherein the oral infection is gingivitis.

Embodiment B173. The use of embodiment B168 or B169, wherein the oral infection is periodontitis.

Embodiment B174. The use of any one of embodiments B168-B173, wherein the subject is a human.

Embodiment B175. The use of any one of embodiments B168-B173, wherein the subject is a mammal.

Embodiment B176. The use of any one of embodiments B168-B173, wherein the subject is a dog.

Embodiment B177. The use of any one of embodiments B168-B173, wherein the subject is a cat.

Embodiment B178. An aqueous hydrogel formulation of any one of embodiments B1-B150 for use in the treatment or prevention of an oral infection in a subject in need thereof.

Embodiment B179. The aqueous hydrogel formulation of embodiment B178, wherein the oral infection is stomatitis.

Embodiment B180. The aqueous hydrogel formulation of embodiment B178, wherein the oral infection is pharyngitis.

Embodiment B181. The aqueous hydrogel formulation of embodiment B178, wherein the oral infection is gingivitis.

Embodiment B182. The aqueous hydrogel formulation of embodiment B178, wherein the oral infection is periodontitis.

Embodiment B183. The aqueous hydrogel formulation of any one of embodiments B178-B182, wherein the subject is a human.

Embodiment B184. The aqueous hydrogel formulation of any one of embodiments B178-B182, wherein the subject is a mammal.

Embodiment B185. The aqueous hydrogel formulation of any one of embodiments B178-B182, wherein the subject is a dog.

Embodiment B186. The aqueous hydrogel formulation of any one of embodiments B178-B182, wherein the subject is a cat.

### Methods of Using Quaternary Ammonium Silane Aqueous Hydrogel Formulations

The quaternary ammonium silane hydrogel formulations described herein can be used to inhibit, prevent, reduce, or treat a microbial infection, including a microbial biofilm An effective amount of a hydrogel formulation as described herein can be used in an amount sufficient to (a) inhibit the progression of an infection; (b) cause a regression of an infection; (c) eliminate an infection; or inhibit or prevent the development of an infection. In an alternative embodiment, an effective amount of a hydrogel formulation as described herein can be used in an amount sufficient to (a) inhibit the detachment and dissemination of pathogenic cells from the EPS matrix; (b) cause a regression of destabilizing overgrowth of pathogenic cells and symbiotic enablers in harmful biofilms; and, (c) inhibit or prevent the attachment and formation of new biofilms by dispersed cells. Accordingly, an effective amount of an active quaternary ammonium silane as provided in a hydrogel formulation described herein will provide a sufficient amount of the active agent when administered to a patient to provide a clinical benefit.

The hydrogel formulation can be administered, for example in a topical application, once a day (q.d.), twice a day (b.i.d.), three times a day (t.i.d.), four times a day (q.i.d.), once every other day (Q2d), once every third day (Q3d), as needed, or any dosage schedule that provides treatment, inhibition, or prevention of a microbial infection.

### Oral Cavity Disorders

In particular aspect, the quaternary ammonium silane aqueous hydrogel formulation is used to treat a disorder within an animal or human oral cavity, for example gingivitis, periodontitis, dental caries, and endodontic abscesses. Bacteria in the oral cavity can cause gingivitis, periodontitis, dental caries, and endodontic abscesses. More than 1 in 5 people have untreated dental caries. Periodontal or gum disease is a pathological inflammatory condition of the gum and bone support (periodontal tissues) surrounding the teeth. The two most common periodontal diseases are gingivitis which is the inflammation of the gum at the necks of the teeth, and periodontitis which is inflammation affecting the bone and tissues of the teeth. Some oral disorders arise from repair of carious teeth wherein the cavity is prepared to receive restoration from an amalgam, gold or other restorative compound. The open cavity is treated with the antimicrobial quaternary ammonium silane hydrogel formulation prior to completing the filling of the tooth in order to reduce the possibility of a biofilm infecting the dentin, producing more carious damage, and in order to strengthen the bond between filling material and the remaining dentin.

In some embodiments, the disorder to be treated is gingivitis. Gingivitis occurs in both chronic and acute forms. Acute gingivitis is usually associated with specific infections, micro-organisms, or trauma. Chronic inflammation of the gum tissue surrounding the teeth is associated with the bacterial biofilm (plaque) that covers the teeth and gums.

In some embodiments, the disorder to be treated is periodontitis. Periodontitis is a chronic inflammatory oral disease that progressively destroys tooth-supporting structures including ligaments and bone.

In some embodiments, a quaternary ammonium silane hydrogel formulation described herein is used to reduce or remove oral cavity microbial populations. In some embodiments, a quaternary ammonium silane hydrogel formulation described herein is provided as a rinse, paste, gel, ointment, or pastille to aid in cleaning teeth, disinfecting cavities, removing plaque, eliminating harmful biofilms, and inhibiting re-infection. Oral cavity-linked bacterial species have been found in bacterial endocarditis, aspiration pneumonia, osteomyelitis, rheumatoid arthritis, and cardiovascular disease. Periodontitis has been associated with an increased risk of oral squamous cell carcinoma. Pathobiology 2021; 88:116-126, “Oral Microbiota and Cancer Development,” DOI: 10.1159/000510979. The main bacterial species found in the oral cavity is Streptococcus, Haemophilus, Lactobacillus acidophilus, Leptrotrichia, Porphyromonas gingivalis, Fusobacterium nucleatum, Prevotella, Propionibacterium, Staphylococcus Veillonella, and Treponema. Ninety-five percent of these bacteria exist as plaque biofilms. Bacterial biofilm infections remain prevalent reasons for implant failure. Fungal infections, Candida spp., papillomavirus (HPV) and Epstein-Barr virus can be involved in oncogeni mulation formation in the oral cavity leading to cancer development. Id. Pseudomembranous candidiasis is common in chronically ill patients and infants. P. gingivalis and F. nucleatum are able to release toxins that enable and maintain constant chronic inflammation. Polymicrobial oral biofilm communities to colonize in the presence of salivary and blood components. The papillated dorsal surface of the tongue and palatal mucosa beneath a maxillary denture are favored reservoir sites.

In some embodiments, a quaternary ammonium silane hydrogel formulation described herein is used topically to treat an oral infection or sore. Topical application includes treatment of mouth/lip care, mouth ulcers and cold sores, including primary oral infection with the virus responsible for cold sores-herpes simplex virus (HSV). After the primary oral infection, HSV may remain inactive only to be activated later as the more common herpes labialis, or “cold sores”. Triggers for reactivation are well known and include sunlight, trauma, tiredness, stress, and menstruation. The most common form of mouth ulcer is called minor aphthous ulceration. Usually, one to five small ulcers appear (less than 1 mm in diameter) on the inside of lips or cheeks, floor of the mouth or tongue. The ulcers tend to be concentrated towards the front of the mouth. Other more serious causes of mouth ulcers include herpes infection.

In certain embodiments, the disorder is periodontitis. In certain embodiments, the disorder is gingivitis. In certain embodiments, the disorder is stomatitis. In certain embodiments, the disorder is pharyngitis. In certain embodiments, the disorder is a human disorder. In certain embodiments, the disorder is an animal disorder.

In certain embodiments, the disorder is a feline dental disease. In yet further embodiments, the disorder is feline stomatitis. In additional embodiments, the disorder is feline pharyngitis. In certain embodiments, the disorder is feline gingivitis. In additional embodiments, the disorder is feline periodontitis. In additional embodiments, the disorder is feline halitosis.

In certain embodiments, the disorder is a canine dental disease. In yet further embodiments, the disorder is canine stomatitis. In additional embodiments, the disorder is canine pharyngitis. In certain embodiments, the disorder is canine gingivitis. In additional embodiments, the disorder is canine periodontitis. In additional embodiments, the disorder is canine halitosis.

In certain embodiments, the disorder is a human dental disease. In yet further embodiments, the disorder is human stomatitis. In additional embodiments, the disorder is human pharyngitis. In certain embodiments, the disorder is human gingivitis. In additional embodiments, the disorder is human periodontitis. In additional embodiments, the disorder is human halitosis.

### Wounds

A particular aspect provided herein is the use of a quaternary ammonium silane hydrogel formulation described herein for the treatment of a wound or burn on a human or an animal. A quaternary ammonium silane hydrogel formulation described herein can be applied directly to the wound. In an alternative aspect, a quaternary ammonium silane hydrogel formulation described herein can be contained in a dressing, foam, wrap, bandage, gauze, film, packing, or other material as described herein, wherein the material is applied to or used to cover and moisturize the wound or burn. In some embodiments, a quaternary ammonium silane hydrogel formulation described herein is contained in a material applied to or used to cover the wound or burn and is released into the wound, for example, in a controlled release manner.

Types of wounds that can be treated include a chronic wound, for example but not limited to, a pressure ulcer, venous ulcer, arterial wound, neuropathic ulcer, diabetic ulcer, for example a lower limbic ulcer or foot ulcer, skin tear, or moisture-associated skin damage (MASD), for example incontinence-associated dermatitis. In some embodiments, the chronic wound is one in which the normal progression of healing has been stalled for over 4 weeks, with continued inflammation, exudation, and granulation tissue but without normal vascularization and epithelialization. In some embodiments, the quaternary ammonium silane hydrogel formulations described herein are used to treat a wound caused by a burn.

Pressure ulcers (also known as pressure injuries) are defined by the National Pressure Ulcer Advisory Panel (NPUAP) as localized damage to the skin and/or underlying soft tissue, usually over a bony prominence or related to a medical or other device. The injury can present as intact skin or an open ulcer and may be painful. The injury results from intense and/or prolonged pressure or pressure combined with shear. The tolerance of soft tissue for pressure and shear may also be affected by microclimate, nutrition, perfusion, comorbidities, and condition of the soft tissue. Pressure ulcers are described according to the NPUAP staging system based on damage that is clinically observed.

Venous ulcers are related to incompetence of the valves of the lower extremities, allowing blood to reflux into the superficial venous system and causing edema. Incomplete emptying of the deep veins can result in higher-than-normal pressure in the peripheral venous system of the lower extremities, which can eventually result in ulcerations.

Arterial wounds result from severe tissue ischemia. One of the most common causes of lower extremity arterial disease and ulceration is atherosclerosis of peripheral arterial vessels. Diabetic foot wounds are also called neuropathic ulcers. Peripheral neuropathy is present in over 80% of patients with foot ulcers. Neuropathy promotes ulcer formation by altering both pain sensation and pressure perception in the foot. Neuropathy can also alter the microcirculation and impair skin integrity. Once wounds occur, healing may be difficult to achieve, especially in patients with deep tissue or bone infections and in those with diminished blood flow to the foot.

A skin tear is defined by the International Skin Tear Advisory Panel (ISTAP) as a traumatic wound caused by mechanical forces (shear, friction, or blunt force), such as the mechanical force required to remove adhesives. Severity may vary by depth but does not extend through the subcutaneous layer. Skin tears are classified based on the degree of skin damage: Type 1: no skin loss; a skin flap can be positioned to cover the exposed wound base; Type 2: partial loss of the skin flap; Type 3: total loss of the skin flap; entire wound bed is exposed.

Moisture-associated skin damage (MASD) is defined as the inflammation and erosion of the skin that accompanies exposure to many different types of corrosive moisture, such as urine, perspiration, and wound drainage. Chronic exposure to moisture macerates the skin, impairing its protective mechanisms and disrupting normal skin flora, which can predispose the patient to cutaneous infections such as candidiasis. Incontinence-associated dermatitis (IAD), a subtype of MASD, is caused by chronic exposure to urine and/or liquid stool.

In another embodiment, the chronic wound comprises an infection which may involve the presence of bacteria, fungi, viruses, amoebas, or a combination of infectious species thereof. In some embodiments, the chronic wound infection is treated by applying a dressing comprising one or more quaternary ammonium silane hydrogel formulations described herein as an antimicrobial composition to the site of infection, wherein the dressing releases one or more quaternary ammonium silanes from the quaternary ammonium silane hydrogel formulations described herein into the site of infection.

### Microbial Infections

In some embodiments, a quaternary ammonium hydrogel formulation as described herein, is used to treat or to prevent a medical disorder which is mediated by the presence of microbes, for example a bacterial infection, fungal infection, or combination of a bacterium and fungal infection.

In some embodiments, a quaternary ammonium silane hydrogel formulation described herein may be used to treat a disorder, typically an infection, caused by a gram-positive bacterium.

Non-limiting example of gram-positive bacteria which may be treated using the quaternary ammonium silane hydrogel formulations described herein either alone or in combination with another therapeutic include: Actinomyces species including Actinomyces israelii, Actinomyces naeslundii, Actinomyces viscosus, Actinomyces odontolyticus, and Actinomyces pyogenes; Bacillus species including Bacillus anthracis, Bacillus cereus, and Bacillus subtilis; Clostridium species including Clostridium botulinum, Clostridium difficile, Clostridium perfringens, Clostridium sordellii, and Clostridium tetani; Corynebacterium species including Corynebacterium diphtheriae, Corynebacterium jeikeium, Corynebacterium minutissimum, Corynebacterium mucifaciens, Corynebacterium pseudotuberculosis, Corynebacterium striatum, Corynebacterium tenuis, and Corynebacterium ulcerans; Enterococcus species including Enterococcus casseliflavus, Enterococcus faecalis, Enterococcus faecium, Enterococcus raffinosus, and Enterococcus hirae; Leuconostoc species including Leuconostoc pseudomesenteroides; Micrococcus species such as Microccocus luteus; Nocardia species including Nocardia asteroids and Nocardia sienata; Propionibacterium species including Propionibacterium acnes; Staphylococcus capitis, Staphylococcus epidermidis, Staphylococcus haemolyticus, Staphylococcus hominis, Staphylococcus lugdunensis, Staphyloccocus pasteuri, and Staphyloccocus saprophyticus; and Streptococcus species including Streptococcus agalactiae, Streptococcus anginosus, Streptococcus bovis, Streptococcus dysgalactiae, Streptococcus mitis, Streptococcus mutans, Streptococcus pneumoniae, Streptococcus pyogenes, Streptococcus sanguinis, Streptococcus suis, and Streptococcus viridans. Staphylococcus species including Staphylococcus aureus,

In some embodiments, a quaternary ammonium silane hydrogel formulation described herein may be used to treat a disorder, typically an infection, caused by a gram-negative bacterium.

Non-limiting examples of gram-negative bacteria which may be treated using the quaternary ammonium silane hydrogel formulations described herein either alone or in combination with another therapeutic include: Acinetobacter species including Acinetobacter baumannii and Acinetobacter iwoffii; Aeromonas species including Aeromonas veronii biovar sobria (previously Aeromonas sobria), Aeromonas caviae, and Aeromonas hydrophila; Alcaligenes/Achromobacter species including Alcaligenes faecalis and Alcaligenes xylosoxidans; Bacteroides species including Bacteroides fragilis; Bartonella species including Bartonella bacilliformis, Bartonella clarridgeiae, Bartonella elizabethae, Bartonella henselae, Bartonella koehlerae, Bartonalla naantalienis, Bartonella quintana, Bartonella rochalimae, Bartonella vinsonii, and Bartonella washoensis; Bordetella species including Bordetella bronchispetica, Bordetella pertussis, and Bordetella parapertussis; Borrelia species including Borrelia afzelii, Borrelia burgdorferi, Borrelia crocidurae, Borrelia duttoni, Borrelia garinii, Borrelia hermsii, Borrelia hispanica, Borellia miyamotoi, Borrelia parkeri, Borrelia persica, Borrelia recurrentis, Borrelia turicatae, and Borrelia venezuelensis; Brevundimonas species including Brevundimonas diminuta and Brevundimonas vesicularis; Brucella species including Brucella abortus, Brucella canis, Brucella melitensis, and Brucella suis; Burkholderia species including Burkholderia cepacia, Burkholderia mallei, and Burkholderia pseudomallei; Campylobacter species including Campylobacter jejuni, Campylobacter coli, Campylobacter upsaliensis, Campylobacter lari, and Campylobacter coli; Chlamydia/Chlamidophila species including Chlamydophila pneumoniae, Chlamydophila psittaci, Chlamidophila pecorum, and Chlamydia trachomatis; Citrobacter species including Citrobacter amalonaticus, Citrobacter freundii, Citrobacter koseri, and Citrobacter diversus; Coxiella burnetti; Ehrlichia species including Ehrlichia canis and Ehrlichia chaffeensis; Enterobacter species including Enterobacter aerogenes and Enterobacter cloacae; Escherichia species including Escherichia coli; Francisella species including Francisella novicida, Francisella philomiragia, and Francisella tularensis; Haemophilus species including Haemophilus influenzae and Haemophilus ducreyi; Helicobacter species including Helicobacter pylori; Klebsiella species including Klebsiella granulomatis, Klebsiella oxytoca, and Klebsiella pneumoniae; Leclercia adecarboxylata; Legionella species including Legionella pneumophila; Leptospira species including Leptospira interrogans, Leptospira noguchii, Leptospira santarosai, and Leptospira weilii; Listeria species including Listeria monocytogenes; Moraxella species including Moraxella catarrhalis, Moraxella lacunata, and Moraxella bovis; Moraxella bovoculi; Morganella species including Morganella morganii; Mycoplasma species including Mycoplasma amphoriforme, Mycoplasma buccale, Mycoplasma faucium, Mycoplasma fermentans, Mycoplasma genitalium, Mycoplasma hominis, Mycoplasma lipophilum, Mycoplasma orale, Mycoplasma penetrans, Mycoplasma pirum, Mycoplasma pneumoniae, Mycoplasma primatum, Mycoplasma salivarium, and Mycoplasma spermatophilum; Neisseria species including Neisseria meningitidis and Neisseria gonorrhoeae; Orientia species including Orientia tsutsugamushi and Orientia chuto; Pantoea species including Pantoea agglomerans; Paracoccus species including Paracoccus yeei; Prevotella species including Prevotella intermedia and Prevotella melaninogenica; Proteus species including Proteus mirabilis, Proteus penneri, and Proteus vulgaris; Providencia species including Providencia rettgeri and Providencia stuartii; Pseudomonas species including Pseucomonas aeruginoas, Pseudomonas oryzihabitans, Pseudomonas plecoglossidica, and Pseudomonas stutzeri; Ralstonia species including Ralstonia pickettii and Ralstonia insidiosa; Rickettsia species including Rickettsia africae, Rickettsia akari, Rickettsia australis, Rickettsia conorii, Rickettsia felis, Rickettsia japonica, Rickettsia prowazekii, Rickettsia rickettsia, Rickettsia sibirica, and Rickettsia typhi; Roseomonas species including Roseomonas gilardii; Salmonella species including Salmonella bongori, Salmonella enterica, Salmonella paratyphi, Salmonella typhi, and Salmonella typhimurium; Serratia species including Serratia marcescens, Serratia liquefaciens, Serratia rubidaea, and Serratia odoriferae; Shigella species including Shigella dysenteriae and Shigella sonnei; Sphingomonas species including Sphingomonas mucosissima and Sphingomonas paucimobilus; Stenotrophomas species including Stenotrophomas maltophilia; Treponema species including Treponema carateum, Treponema paraluiscuniculi, and Treponema pallidum; Ureaplasma species including Ureaplasma urealyticum; Vibrio species including Vibrio cholera, Vibrio parahaemolyticus, and Vibrio vulnificus; and Yersinia species including Yersinia enterocolitica, Yersinia pestis, and Yersinia pseudotuberculosis. Additional examples of gram-negative pathogens include Porphyromonas gingivalis, Treponema denticola, fusobacterium nucleate, and Prevotella intermedia.

Non-limiting examples of disorders mediated by a bacterium that may be treated by a quaternary ammonium silane hydrogel formulation described herein, either alone or in combination with another therapeutic, include actinomycosis, anaplasmosis, anthrax, bacillary angiomatosis, actinomycetoma, bacterial conjunctivitis, bacterial keratitis, bacterial pneumonia, bacterial vaginosis, bacterial endocarditis, bartonellosis, botulism, boutenneuse fever, brucellosis, bejel, brucellosis spondylitis, bubonic plague, Buruli ulcer, Bairnsdale ulcer, bacillary dysentery, campylobacteriosis, Carrion's disease, cat-scratch disease, cellulitis, chancroid, chlamydia, chlamydia conjunctivitis, clostridial myonecrosis, cholera, Clostridium difficile colitis, diphtheria, Daintree ulcer, donavanosis, dysentery, erhlichiosis, epidemic typhus, fried rice syndrome, five-day fever, floppy baby syndrome, Far East scarlet-like fever, gas gangrene, glanders, gonorrhea, granuloma inguinale, human necrobacillosis, necrotizing fasciitis, hemolytic-uremic syndrome, human ewingii ehrlichiosis, human monocytic ehrlichiosis, human granulocytic anaplasmosis, infant botulism, Izumi fever, Kawasaki disease, Kumusi ulder, lymphogranuloma venereum, Lemierre's syndrome, Legionellosis, leprosy, leptospirosis, listeriosis, Lyme disease, lymphogranuloma venereum, Malta fever, Mediterranean fever, myonecrosis, mycoburuli ulcer, mucocutaneous lymph node syndrome, meliodosis, meningococcal disease, murine typhus, Mycoplasma pneumonia, mycetoma, neonatal conjunctivitis, nocardiosis, Oroya fever, ophthalmia neonatorum, ornithosis, Pontiac fever, peliosis hepatis, pneumonic plague, postanginal shock including sepsis, pasteurellosis, pelvic inflammatory disease, pertussis, plague, pneumococcal infection, pneumonia, psittacosis, parrot fever, pseudotuberculosis, Q fever, quintan fever, rabbit fever, relapsing fever, rickettsialpox, Rocky Mountain spotted fever, rat-bite fever, Reiter syndrome, rheumatic fever, salmonellosis, scarlet fever, sepsis, septicemic plague, Searls ulcer, shigellosis, soft chancre, syphilis, streptobacillary fever, scrub typhus, Taiwan acute respiratory agent, Trench fever, trachoma, tuberculosis, tularemia, typhoid fever, typhus, tetanus, toxic shock syndrome, undulant fever, ulcus molle, Vibrio parahaemolyticus enteritis, Whitmore's disease, walking pneumonia, Waterhouse-Friderichsen syndrome, yaws, and yersiniosis.

In some embodiments, the infection to be treated by a quaternary ammonium silane hydrogel formulation described herein is composed of pathogenic fungi. Pathogenic fungi can also adhere to abiotic surfaces such as prostheses and catheters; in particular, yeasts take advantage of this condition to gain access to blood circulation, reaching the internal organs of patients. This is alarming, as disseminated fungal infections have a high mortality rate. (Verstrepen K. J., Klis F.M. Flocculation, adhesion and biofilm formation in yeasts. Mol. Microbiol. 2006, 60, 5-15; doi: 10.1111/j.1365-2958. 2006. 05072.x)

In some embodiments, a quaternary ammonium silane hydrogel formulation described herein targets a fungal biofilm. Candida albicans is the most studied model of biofilm formation and shows distinct phases of development that are similar to those of bacterial biofilms. Paracoccidioides brasiliensis is a dimorphic fungus responsible for paracoccidioidomycosis, a systemic mycosis endemic in Latin America. Sardi et al. (Sardi Jde C., Pitangui Nde S., Voltan A. R., Braz J. D., Machado M. P., Fusco Almeida A. M., Mendes Giannini M. J. In vitro Paracoccidioides brasiliensis biofilm and gene expression of adhesins and hydrolytic enzymes. Virulence. 2015, 6, 642-651. doi: 10.1080/21505594.2015.1031437.). Histoplasma capsulatum biofilm was first described by Pitangui et al. This fungus also features thermal dimorphism and is the cause of histoplasmosis, a respiratory and systemic mycosis whose evolution depends on the survival and replication of yeast in alveolar macrophages (Pitangui N. S., Sardi J. C., Silva J. F., Benaducci T., Moraes da Silva R. A., Rodriguez-Arellanes G., Taylor M. L., Mendes-Giannini M. J., Fusco-Almeida A. M. Adhesion of Histoplasma capsulatum to pneumocytes and biofilm formation on an abiotic Biofouling. surface. 2012, 28, 28, 711-718. doi: 10.1080/08927014.2012.703659).

In some embodiments, a quaternary ammonium silane hydrogel formulation described herein is used in treating an infection caused by a dermatophyte. Dermatophytes are fungi that invade keratinized tissues producing dermatophytosis, one of the most common dermatomycoses in human and animals (Weitzman I., Summerbell R.C. The dermatophytes. Clin. Microbiol. Rev. 1995, 8, 240-259. doi: 10.1016/S0733-8635 (05) 70320-X). Among dermatophytosis, onychomycosis often relapses and involves long, sometimes ineffective treatment. Given this context and the hypothesis of Burkhart et al., which states that biofilm formation by dermatophytes can explain dermatophytomas, Costa-Orlandi et al., confirmed in vitro biofilm formation by two of the most prevalent species worldwide: Trichophyton rubrum and T. mentagrophytes (Burkhart C. N., Burkhart C. G., Gupta A. K. Dermatophytoma: Recalcitrance to treatment because of existence of fungal biofilm. J. Am. Acad. Dermatol. 2002, 47, 629-631. doi: 10.1067/mjd.2002.124699; Costa-Orlandi C.B., Sardi J. C., Santos C. T., Fusco-Almeida A. M., Mendes-Giannini M.J. In vitro characterization of Trichophyton rubrum and T. mentagrophytes biofilms. Biofouling. 2014, 30, 719-727. doi: 10.1080/08927014.2014.919282).

In some embodiments, a quaternary ammonium silane hydrogel formulation described herein is used in treating an infection caused by Candida auris. Candida auris is an emerging yeast that causes healthcare-associated infections. It is the first Candida species to show resistance to all three major classes of antifungals. See, e.g., Sansom S, et al. Abstract 50. Presented at: Society for Healthcare Epidemiology of America Spring Meeting; Apr. 12-14, 2022. (. auris is also commonly found in biofilms on the body with other multidrug-resistant organisms and is more commonly found with resistant gram-positive organisms such as MRSA and vancomycin-resistant Enterococcus. C auris has been shown to be common on hospital surfaces. Id.

In some embodiments, one or more of the quaternary ammonium silane hydrogel formulations as described herein, are used to treat or to prevent a medical disorder which is mediated by the presence of a fungus or algae, for example a fungal infection.

Non-limiting examples of algae and fungi which may be treated using quaternary ammonium silane hydrogel formulations described herein either alone or in combination with another therapeutic include: Absidia species including Absidia corymbifera; Alterania species including Alterania alternate; Aspergillus species including Aspergillus clavatus, Aspergullus flavus, Aspergillus fumigatus, Aspergillus niger, Aspergillus sydowii, Aspergillus terreus, Aspergillus versicolor, and Aspergillus verrucaria; Aureobasidium species including Aureobasidium pullans; Batrachochytrium species including Batrachochytrium dendrobatidis and Batrachochytrium salamandrivorans; Blastomyces species including Blastomyces dermatitidis; Candida species including Candida albicans, Candida auris, Candida dubliniensis, Candida glabrata, Candida parapsilosis, Candida rugosa, and Candida tropicalis; Chaetomium species including Chaetomium globsum; Cladosporium species including Cladosporium cladosporoides; Chlorophyta, Coccidioides species including Coccidioides immitis and Coccidioides posadasii; Cryptococcus species including Cryptococcus albidus, Cryptococcus gattii, Cryptococcus laurentii, Cryptococcus neoformans, and Cryptococcus uniguttulatus; Cunninghamella species; Curvularia species including Curvularia brachyspora, Curvularia clavata, Curvularia geniculata, Curvularia lunata, Curvularia pallescens, Curvularia senegalensis, and Curvularia verruculosa; Cyanophyta Chrysophyta, Dreschslera species including Dreschlera australiensis; Epidermophyton species including Epidermophyton floccosum; Fonsecaea species including Fonsecaea compacta and Fonsecaea pedrosoi; Fusarium species including Fusarium solani, Fusarium oxysporum, and Fusarium chlamydosporum, Geotrichum species including Geotrichum capitatum, Geotrichum candidum, and Geotricum clavatum; Gliomastix species including Gliomastix cerealis; Gloeophyllum species including Gloeophyllum trabeum; Histoplasma species including Histoplasma capsulatum and Histoplasma capsulatum var. faciminosum; Malassezia species including Malassezia furfur and Malassezia globosa; Microsporum species; Monilia species including Monilia grisea, Mucor species including Mucor indicus; Paracoccidioides species including Paracoccidioides brasiliensis; Penicillium species; Piedraia species including Piedraia hortae and Piedraia quintanilhae; Phialophora species including Phialophora verrucosa; Phoma species including Phoma fimeti; Pithomyces species including Pithomyces chartarum; Pneumocystis species including Pneumocystis carinii and Pneumocystis jirovecii; Poria species including Poria placenta; Rhizopus species including Rhizopus microspores, Rhizopus oryzae, and Rhizopus stolonifer; Scolecobasidium species including Scolecobasidium humicola; Sporothrix species including Sporothrix brasiliensis, Sporothrix globosa, and Sporothrix schenckii; and Trichoderma species including Trichoderma viride; and Trichophyton species including Trichosporon beigelii, Trichophyton concentricum, Trichophyton interdigitale, Trichophyton mentagrophytes, Trichophyton rubrum, and Trichophyton tonsurans.

Non-limiting examples of disorders mediated by a fungus that may be treated by quaternary ammonium silane hydrogel formulations described herein, either alone or in combination with another therapeutic, include invasive aspergillosis, black piedra, blastomycosis, oropharyngeal candidiasis, vulvovaginal candidiasis, chromoblastomycosis, chytridiomycosis, coccidioimycosis, cryptococcosis, dermatophytosis, fusariosus, geotrichosis, histoplasmosis, mucormycosis, mycetoma, paracoccidioidomycosis, pneumocystis pneumonia, sporotrichosis, Tinea barbae, Tinea capitis, Tinea corporis, Tinea cruris, Tinea manum, Tinea nigra, Tinea unguium, Tinea versicolor, white piedra, and zygomycosis.

In some embodiments, quaternary ammonium silane hydrogel formulations described herein may be used to treat a disorder, typically an infection, caused by a mycobacterium. Non-limiting examples of mycobacteria which may be treated using a quaternary ammonium silane hydrogel formulation described herein either alone or in combination with another therapeutic include Mycobacterium abcessus, Mycobacterium africanum, Mycobacterium agri, Mycobacterium aichiense, Mycobacterium alvei, Mycobacterium arabiense, Mycobacterium aromaticivorans, Mycobacterium arosiense, Mycobacterium arupense, Mycobacterium aquaticum, Mycobacterium asiaticum, Mycobacterium aubagnese, Mycobacterium aurum, Mycobacterium austroafricanum, Mycobacterium avium, Mycobacterium avium paratuberculosis, Mycobacterium avium silvaticum, Mycobacterium avium hominussuis, Mycobacterium bacteremicum, Mycobacterium barrassiae, Mycobacterium boenickei, Mycobacterium bohemicum, Mycobacterium bolletii, Mycobacterium botniense, Mycobacterium bovis, Mycobacterium branderi, Mycobacterium brisbanense, Mycobacterium brumae, Mycobacterium canariasense, Mycobacterium canettii, Mycobacterium caprae, Mycobacterium chimaera, Mycobacterium chelonae, Mycobacterium chitae, Mycobacterium chubuense, Mycobacterium colombiense, Mycobacterium conceptionense, Mycobacterium confluentis, Mycobacterium conspicuum, Mycobacterium cookii, Mycobacterium cosmeticum, Mycobacterium diernhoferi, Mycobacterium doricum, Mycobacterium duvalii, Mycobacterium elephantis, Mycobacterium fallax, Mycobacterium farcinogenes, Mycobacterium flavescens, Mycobacterium florentinum, Mycobacterium fortuitum, Mycobacterium frederikbergense, Mycobacterium gadium, Mycobacterium gastri, Mycobacterium genavense, Mycobacterium gilvum, Mycobacterium gordonae, Mycobacterium haemophilusobacterium hassiacum, Mycobacterium heidelbergense, Mycobacterium heckshornense; Mycobacterium hiberniae, Mycobacterium hodleri, Mycobacterium holsaticum, Mycobacterium houstonense, Mycobacterium icosiumassilensis, Mycobacterium immunogenum, Mycobacterium indicus pranii, Mycobacterium intacellulare, Mycobacterium intracellulare, Mycobacterium interjectum, Mycobacterium intermedium, Mycobacterium iranicum, Mycobacterium kansasii, Mycobacterium komossense, Mycobacterium kubicae, Mycobacterium lentiflavum, Mycobacterium leprae, Mycobacterium lepraemurium, Mycobacterium lepromatosis, Mycobacterium liflandii, Mycobacterium llatzerense, Mycobacterium madagascariense, Mycobacterium mageritense, Mycobacterium malmoense, Mycobacterium marinum, Mycobacterium massiliense, Mycobacterium massilipolynesiensis, Mycobacterium microti, Mycobacterium monacense, Mycobacterium montfiorense, Mycobacterium morokaense, Mycobacterium mucogenicum, Mycobacterium mungi, Mycobacterium murale, Mycobacterium nebraskense, Mycobacterium neoaurum, Mycobacterium neworleansense, Mycobacterium nonchromogenicum, Mycobacterium obuense, Mycobacterium orygis, Mycobacterium palustre, Mycobacterium parascofulaceum, Mycobacterium parafortuitum, Mycobacterium perigrinum, Mycobacterium phlei, Mycobacterium phocaicum, Mycobacterium pinnipedii, Mycobacterium porcinum, Mycobacterium pseudoshottsii, Mycobacterium psychotolerans, Mycobacterium pulveris, Mycobacterium pyrenivorans, Mycobacterium saskatchewanense, Mycobacterium sediminis, Mycobacterium senegalense, Mycobacterium septicum, Mycobacterium shimoidei, Mycobacterium shottsii, Mycobacterium simiae, Mycobacterium smegmatis, Mycobacterium sphagni, Mycobacterium stephanolepidis, Mycobacterium suricattae, Mycobacterium szulgai, Mycobacterium talmoniae, Mycobacterium terrae, Mycobacterium thermoresistibile, Mycobacterium triplex, Mycobacterium triviale, Mycobacterium tuberculosis, Mycobacterium tusciae, Mycobacterium ulcerans, Mycobacterium vaccae, Mycobacterium vanbaalenii, Mycobacterium xenopi, and Mycobacterium yongonense.

In some embodiments, the quaternary ammonium silane hydrogel formulations as described herein, are used to treat or to prevent a medical disorder which is mediated by the presence of amoeba, for example an amoebal infection.

Non-limiting examples of amoeba which may be treated using quaternary ammonium silane hydrogel formulations described herein either alone or in combination with another therapeutic include: Acanthamoeba species; Balamuthia species including Balamuthia mandrillaris; Dientamoeba species including Dientamoeba fragilis; Endolimax species including Endolimax nana; Entamoeba species including Entamoeba Bangladeshi, Entamoeba coli, Entamoeba dispar, Entamoeba gingivalis, Entamoeba hartmanni, Entamoeba histolytica, Entamoeba moshkovskii, and Entamoeba polecki; Iodamoeba species including Iodamoeba butschlii; Naegleria species including Naegleria fowleri; and Sappinia species including Sappinia diploidea and Sappinia pedata.

Non-limiting examples of disorders mediated by an amoeba that may be treated by quaternary ammonium silane hydrogel formulations described herein, either alone or in combination with another therapeutic, include amoebiasis, amoebic dysentery, amoebic liver abscess, cutaneous amoebiasis, amoebic brain abscess, amebiasis cutis, Acanthamoeba keratitis, cutaneous acanthamoebiasis, granulomatous amoebic encephalitis, Balamuthia amoebic encephalitis, and Sappinia amoebia encephalitis.

In some embodiments, the infection is caused by Acinetobacter species, Aspergillus species, Burkholderia cepacia complex, Campylobacter species, Candida species, Clostridium difficile, Coccidioides species, Cryptococcus species, Enterobacteriaceae, Enterococcus species, Helicobacter pylori, Mycobacterium tuberculosis complex, Neisseria gonorrhoeae, Neisseria meningitidis, Non-tuberculous mycobacteria species, Pseudomonas species, Staphylococcus aureus, Streptococcus agalactiae, Streptococcus pneumoniae, Streptococcus pyogenes, and Vibrio cholerae.

In certain alternative embodiments, the infection is caused by Staphylococcus aureus. Staphylococcus epidermidis, Pseudomonas aeruginosa, Streptococcus pyogenes, Candida albicans, Candida auris, Cladosporium herbarum, Aspergillus niger, Proteus mirabilis, Klebsiella pneumoniae, Acinetobacter baumanii, an Enterobacter spp. or a Fusarium spp.

In some embodiments, a quaternary ammonium silane hydrogel formulation described herein is used to treat a skin infection in a host, for example a human. The infection may be caused by a bacterium, a fungus, an amoeba, or a virus as described herein.

In another embodiment, the method is used to treat a skin infection in another mammal, for example a cat, a dog, a cow, a pig, or a horse.

Examples of bacterial cutaneous infections that may be treated by a quaternary ammonium silane hydrogel formulation described herein include, but are not limited to: acne vulgaris, African tick bite fever; American tick bite fever (Rickettsia parkeri infection); Bacillary angiomatosis; Bejel (endemic syphilis); Blastomycosis-like pyoderma (pyoderma vegetans); Blistering distal dactylitis; Botryomycosis; Brill-Zinsser disease; Brucellosis (Bang's disease, Malta fever, undulant fever); Bubonic plague; Bullous impetigo; Campylobacter jejuni; Cat scratch disease (cat scratch fever, English-Wear infection, inoculation lymphoreticulosis, subacute regional lymphadenitis); Cellulitis; Chancre; Chancroid (soft chancre, ulcus molle); Chronic lymphangitis; Chronic recurrent erysipelas; Chronic undermining burrowing ulcers (Meleney gangrene); Condylomata lata; Cutaneous actinomycosis; Dermatitis gangrenosa (gangrene of the skin); Ecthyma; Ecthyma gangrenosum; Elephantiasis nostras; Endemic typhus (murine typhus); Endometritis; Epidemic typhus (epidemic louse-borne typhus); Erysipelas (ignis sacer, Saint Anthony's fire); Erysipeloid of Rosenbach; Erythema marginatum; Erythrasma; Felon; Flea-borne spotted fever; Flinders Island spotted fever; Flying squirrel typhus; Folliculitis; Fournier gangrene (Fournier gangrene of the penis or scrotum); Furunculosis (boil); Gas gangrene (clostridial myonecrosis, myonecrosis); Glanders (equinia, farcy, malleus); Gonococcemia (arthritis-dermatosis syndrome, disseminated gonococcal infection); Gonorrhea (clap); Gram-negative folliculitis; Gram-negative toe web infection; Granuloma inguinale (Donovanosis, granuloma genitoinguinale, granuloma inguinale tropicum, granuloma venereum, granuloma venereum genitoinguinale, lupoid form of groin ulceration, serpiginous ulceration of the groin, ulcerating granuloma of the pudendum, ulcerating sclerosing granuloma); Green nail syndrome; Hospital furunculosis; Hot tub folliculitis (Pseudomonas aeruginosa folliculitis); Human granulocytotropic anaplasmosis; Human monocytotropic ehrlichiosis; Impetigo contagiosa; Japanese spotted fever; Leptospirosis (Fort Bragg fever, pretibial fever, Weil's disease); Listeriosis; Ludwig's angina; Lupoid sycosis; Lyme disease (Afzelius' disease, Lyme borreliosis); Lymphogranuloma venereum (climatic bubo, Durand-Nicolas-Favre disease, lymphogranuloma inguinale, poradenitis inguinale, strumous bubo); Malakoplakia (malacoplakia); Mediterranean spotted fever (Boutonneuse fever); Melioidosis (Whitmore's disease); Meningococcemia; Missouri Lyme disease; Necrotizing fasciitis (flesh-eating bacteria syndrome); Neonatal toxic shock-like exanthematous disease; Noma neonatorum; North Asian tick typhus; Ophthalmia neonatorum; Oroya fever (Carrion's disease); Perianal cellulitis (perineal dermatitis, streptococcal perianal disease); Periapical abscess; Persistent mating-induced endometritis (PMIE); Pinta; Pitted keratolysis (keratolysis plantare sulcatum, keratoma plantare sulcatum, ringed keratolysis); Plague; Primary gonococcal dermatitis; Pseudomonal Pyoderma; Pseudomonas hot-foot syndrome; Pyogenic paronychia; Pyomyositis; Q fever; Queensland tick typhus; Rat-bite fever; Recurrent toxin-mediated perineal erythema; Rhinoscleroma; Rocky Mountain spotted fever; Scarlet fever; Scrub typhus (Tsutsugamushi fever); Shigellosis; Staphylococcal scalded skin syndrome (pemphigus neonatorum, Ritter's disease); Streptococcal intertrigo; Superficial pustular folliculitis (impetigo of Bockhart, superficial folliculitis); Sycosis vulgaris (barber's itch, sycosis barbae); Syphilid; Syphilis (lues); Tick-borne lymphadenopathy; Toxic shock syndrome (streptococcal toxic shock syndrome, streptococcal toxic shock-like syndrome, toxic streptococcal syndrome); Trench fever (five-day fever, quintan fever, urban trench fever); Tropical ulcer (Aden ulcer, jungle rot, Malabar ulcer, tropical phagedena); Tularemia (deer fly fever, Ohara's disease, Pahvant Valley plague, rabbit fever); Verruga peruana; and Yaws (bouba, frambösie, parangi, pian).

Examples of mycobacterial cutaneous infections that may be treated by a quaternary ammonium silane hydrogel formulation described herein include, but are not limited to: Aquarium granuloma (fish-tank granuloma, swimming-pool granuloma); Borderline lepromatous leprosy; Borderline leprosy; Borderline tuberculoid leprosy; Buruli ulcer (Bairnsdale ulcer, Searl ulcer, Searle's ulcer); Erythema induratum (Bazin disease); Histoid leprosy; Lepromatous leprosy; Leprosy (Hansen's disease); Lichen scrofulosorum (tuberculosis cutis lichenoides); Lupus vulgaris (tuberculosis luposa); Miliary tuberculosis (disseminated tuberculosis, tuberculosis cutis acuta generalisata, tuberculosis cutis disseminata); Papulonecrotic tuberculid; Primary inoculation tuberculosis (cutaneous primary complex, primary tuberculous complex, tuberculous chancre); Scrofuloderma (tuberculosis cutis colliquativa); Tuberculosis cutis orificialis (acute tuberculous ulcer, orificial tuberculosis); Tuberculosis verrucosa cutis (lupus verrucosus, prosector's wart, warty tuberculosis); Tuberculous cellulitis; Tuberculous gumma (metastatic tuberculous abscess, metastatic tuberculous ulcer); and Tuberculoid leprosy.

Examples of fungal cutaneous infections that may be treated by a quaternary ammonium silane hydrogel formulation described herein include, but are not limited to: African histoplasmosis; Alternariosis; Antibiotic candidiasis (iatrogenic candidiasis); Black piedra; Candida auris, Candidal intertrigo; Candidal onychomycosis; Candidal paronychia; Candidal vulvovaginitis; Candidid; Chromoblastomycosis (chromomycosis, cladosporiosis, Fonseca's disease, Pedroso's disease, phaeosporotrichosis, verrucous dermatitis); Chronic mucocutaneous candidiasis; Coccidioidomycosis (California disease, desert rheumatism, San Joaquin Valley fever, valley fever); Congenital cutaneous candidiasis; Cryptococcosis; Dermatophytid; Diaper candidiasis; Disseminated coccidioidomycosis (coccidioidal granuloma); Distal subungual onychomycosis; Entomophthoromycosis; Erosio interdigitalis blastomycetica; Favus; Fungal folliculitis (majocchi granuloma); Fusariosis; Geotrichosis; Granuloma gluteale infantum; Histoplasmosis (cave disease, Darling's disease, Ohio Valley disease, reticuloendotheliosis); Hyalohyphomycosis; Kerion; Lobomycosis (keloidal blastomycosis, lacaziosis, Lobo's disease); Mucormycosis; Mycetoma (Madura foot, maduromycosis); North American blastomycosis (blastomycetic dermatitis, blastomycosis, Gilchrist's disease); Onychomycosis (dermatophytic onychomycosis, ringworm of the nail, tinea unguium); Oral candidiasis (thrush); Otomycosis; Perianal candidiasis; Perlèche (angular cheilitis); Phaeohyphomycosis; Piedra (trichosporosis); Pityrosporum folliculitis; Primary cutaneous aspergillosis; Primary cutaneous coccidioidomycosis; Primary cutaneous histoplasmosis; Primary pulmonary coccidioidomycosis; Primary pulmonary histoplasmosis; Progressive disseminated histoplasmosis; Proximal subungual onychomycosis; Rhinosporidiosis; South American blastomycosis (Brazilian blastomycosis, paracoccidioidal granuloma, paracoccidioidomycosis); Sporotrichosis (rose-gardener's disease); Systemic candidiasis; Tinea barbae (barber's itch, ringworm of the beard, tinea sycosis); Tinea capitis (herpes tonsurans, ringworm of the hair, ringworm of the scalp, scalp ringworm, tinea tonsurans); Tinea corporis (ringworm, tinea circinata, tinea glabrosa); Tinea corporis gladiatorum; Tinea cruris (crotch itch, eczema marginatum, gym itch, jock itch, ringworm of the groin); Tinea faciei; Tinea imbricate (tokelau); Tinea incognito; Tinea manuum; Tinea nigra (superficial phaeohyphomycosis, tinea nigra palmaris et plantaris); Tinea pedis (athlete's foot, ringworm of the foot); Tinea versicolor (dermatomycosis furfuracea, pityriasis versicolor, tinea flava); White piedra; White superficial onychomycosis; and Zygomycosis (phycomycosis).

### Dressings and the Like

In some embodiments, the quaternary ammonium silane hydrogel formulation described herein is dispersed in a suitable dressing. The dressing that is chosen should allow release of the desired quaternary ammonium silane hydrogel formulation over a period of time dependent upon the desired application. The dressing can be wetted before placement at the site of infection by saturation with the quaternary ammonium silane hydrogel formulation, even though it may be additionally moistened due to exudate at the site of infection. Alternatively, the dressing can be placed at the site of a wound and/or infection and subsequently saturated with the quaternary ammonium silane hydrogel formulation, for example by application of the quaternary ammonium silane hydrogel formulation by dropper or syringe, or other suitable means. In alternative embodiments, a quaternary ammonium silane hydrogel formulation described herein is dispersed in a suitable dressing, wherein at least a portion of the antimicrobial composition remains in order to, for example, reduce odor, to confine the spread or prevent the spread of microbes on the dressing, or to protect a wound or other covering from contamination.

Additionally, the dressing may comprise additional additives that ensure the maintenance of a moist environment at the site of infection. The dressing must be composed of a material that is hypoallergenic and non-toxic in order to be acceptably applied to a living host. In some embodiments, the dressing would absorb the quaternary ammonium silane hydrogel formulation and would then subsequently release the quaternary ammonium silane hydrogel formulation once placed in the site of infection involving a biofilm.

In certain embodiments, the dressing has a bulk density that is low enough to allow the quaternary ammonium silane hydrogel formulation to be incorporated within, but high enough to provide sufficient structural integrity. The dressing should be porous to provide sufficient intercalation of the quaternary ammonium silane hydrogel formulation among the material, allowing space for sufficient wetting with the quaternary ammonium silane hydrogel formulation along with efflux into the site of treatment. The level of porosity of the dressing should be high enough to allow sufficient wetting with the quaternary ammonium silane hydrogel formulation, but should still allow for the dressing to have sufficient material strength.

In certain embodiments, the dressing is fashioned from a flexible polymeric material that provides sufficient porosity but still provides structural integrity in the desired application. In certain embodiments, two or more of the dressing components may be combined, as deemed necessary for the particular application, into a composite material. In some cases, the two or more components may be present in layers. In other cases, the two or more components may be impregnated or intercalated into each other. The combination of dressing components may be necessary for structural integrity to ensure placement, positioning, and functioning at the site of infection.

In some embodiments, the dressing comprises a polymer foam, for example a conformable foam. The polymer foam may allow release of the desired quaternary ammonium silane hydrogel formulation by either diffusion, ionic interactions, or by degradation of the material composition of the dressing.

In some embodiments, the polymer foam can absorb exudate that may occur due to infection involving the presence of a biofilm. In some embodiments, the polymer foam is biodegradable or non-degradable, depending upon the intended use within the patient.

Examples of materials suitable for the formation of a polymer foam include, but are not limited to, cellulose and cellulose derivatives, microcrystalline cellulose, calcium alginate, polyacrylic acid, polyethylene glycol, polypropylene glycol, divinyl glycol, polyethylene oxide, polypropylene oxide, carboxymethyl cellulose, hydroxyethyl cellulose, polylactide, polyglycolide, polymethacrylic acid, poly-y-benzyl-L-glutamate, polypropylene fumarate, poly-ε-caprolactone, poly-butylene terephthalate, polyvinyl alcohol, polyvinyl ether, poly-1-vinyl-2-pyrrolidinone, 2,5 dimethyl-1,5-hexadiene, divinyl benzene, polystyrene-divinyl benzene, polyanhydrides such as polybis (p-carboxy-phenoxy)propane-co-sebacic acid, polyhydroxyalkanoates such as poly-β hydroxybutyrate or poly-β-butyrolactone, and alkyl-substituted silica gel formed from reagents such as an quaternary ammonium silane and dimethyldiethoxysilane. In preferred embodiments, the polymer foam is composed of polyurethane.

In another embodiment, the polymer foam is composed of cellulose. In yet another embodiment, the polymer foam is composed of calcium alginate.

In some embodiments, the dressing comprises a fabric composition. The fabric composition may be composed of fibers including natural fibers, synthetic fibers, cellulose, woven or nonwoven fabric material, gauze material, or mixtures thereof. Examples of acceptable fibers include, but are not limited to, cotton, polyester, wool, silk, and rayon. The fabric composition may have varying levels of absorbency depending on the desired application. The fabric composition may additionally be coated with an appropriate polymer composition that effects absorbance and dispersion of the active quaternary ammonium silane or additional additives.

In some embodiments, the dressing additionally comprises a polymeric film. A polymeric film may be desirable to ensure proper sealing of the dressing to prevent the entry of dirt and debris and to maintain moisture at the treatment site. In preferred embodiments, the polymeric film comprises an adhesive side that adheres to the edges of the site of the infection to provide a seal and a non-adhesive side. In some embodiments, the polymeric film is composed of polyurethane.

In certain embodiments, the dressing is self-adhesive.

In some embodiments, the dressing additionally comprises a collagen matrix. A collagen matrix may be included in applications where it would be deemed desirable, such as providing a template in wound healing. The collagen matrix may be present as an ointment, gel, pad, paste, or sheet. The collagen matrix may be derived from a bovine, porcine, equine, or avian source. The collagen matrix may be composed of type I, II, III, IV, or V collagen.

In some embodiments, the collagen matrix may interact with the site of infection caused by a biofilm by forming a gel. Additional macromolecular structures, such as hyaluronic acid or hyaluronan, fibronectin, laminin, proteoglycans and mixtures thereof, may be incorporated into the collagen matrix. In some embodiments, the collagen matrix is chemically cross-linked.

In some embodiments, the dressing is composed of a dissolvable material. A dressing composed of a dissolvable material can allow for the dressing to be placed in the site of a wound and/or infection without the need for retrieval upon completion of the treatment. In certain embodiments, the dressing comprises a polymeric material comprises thermoplastic polymer, thermosetting polymer, biodegradable polymer, modified polymers, crosslinked polymers, polymers for controlled delivery), hydrogels, hydrocolloids, liquid forming polymer, gel forming polymer, silicone-based polymeric material, film forming polymer, adhesive polymer, polymers for controlled delivery copolymers, polymers for medical uses, or mixtures thereof; fabric material, nonadherent dressing material or hydrofibers.

In certain embodiments, the dressing is hydrofiber. Hydrofibers are soft, sterile, non-woven pad or ribbon dressing composed of sodium carboxymethylcellulose, which is incorporated in the form of a fleece held together by a needle-bonding process. This conformable material can absorb a large amount of wound fluid, such as exudate with bacteria. This is then transformed into a soft gel, which creates a moist environment to support the body's healing process. The gel also aids the removal of non-viable tissue from the wound (autolytic debridement), without damaging newly formed tissue. Hydrofibers are neither hydrocolloids nor alginates, but a separate category incorporating the benefits of both. (Thomas S. Sodium Carboxymethylcellulose Primary Wound

Dressing, Aquacel. Available accessed 22 Oct. 2010)

In certain embodiments, the nonadherent dressing material comprises a nonadherent fabric material, for example, nonadherent gauze.

In certain embodiments, the dressing is used for covering wounds or wound care applications, infection or exposed skin. The wound for example, can be due to an infection, burn, exposed skin, open wound, skin lacerations, abrasion's, punctures, avulsions, scabs, surgical wounds, abscesses, skin tears, skin ulcers or lesions, damaged tissue, bites, moisture associated skin damage, foot ulcers, necrosis, nonhealing wounds, compromised skin grafts or flaps, acute wounds, chronic wounds, trauma, and other skin exposure related injuries.

Examples include but not limited to, fiber mats gauze (cotton, yarn, natural or synthetic fibers, nonwoven, blends thereof, and the like), cotton balls, tulle, bandages (liquid bandage), adhesive, tissue adhesive, bio-adhesives, tapes, sheets, labels, liners, rolls of film and sheets, tear-apart sheets, rolls, fabric materials described herein, and the like.

### EXAMPLES OF THE PRESENT INVENTION

The following example illustrates various aspects of the disclosure. It will be apparent to those skilled in the art that many modifications, both to materials and methods, may be practiced without departing from the scope of the disclosure.

### Synthesis of Compounds of the Invention

### Example 1. Dimethyl-octadecyl-[3-[tris(2-hydroxypropoxy) silyl]propyl]ammonium chloride

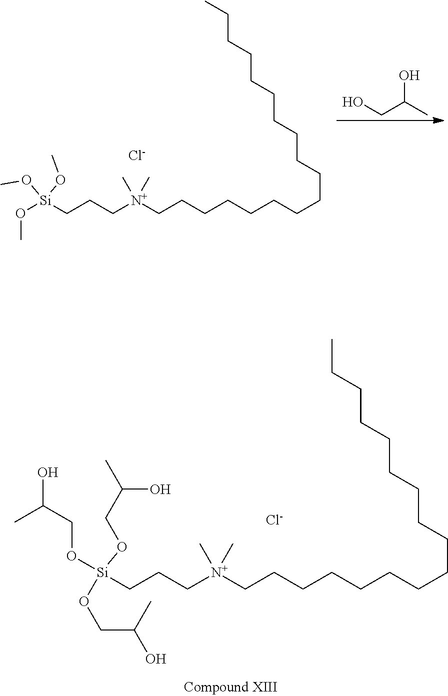

A 1 L round bottom reaction vessel was outfitted with a heating mantle, stir bar, downward condenser, receiving flask, oil bubbler, a thermoprobe for the pot, and a thermoprobe for the head.

The reactor was charged with 36 g of dimethyloctadecyl[3-(trimethoxysilyl)propyl]ammonium chloride, 16.4 g of 1,2-propanediol and ˜300 mL of DMF. The mixture was heated to 145° C. (pot temp) and the methanol was collected until the head temp drops and no more MeOH evolved. The reaction was then cooled and DMF evaporated at reduced pressure. Toluene (250 ml) was added, stirred at room temp for 1 hour, and then evaporated at reduced pressure. Acetonitrile (250 ml) was added, stirred at room temp for 1 hour, and then evaporated at reduced pressure. The resultant product was placed under high vac overnight to yield an off-white viscous liquid (44 g; 99% yield). The NMR was consistent with the proposed structure. The product dissolved readily in water and was stable for weeks.

### Example 2. N,N-Dimethyl-N-(3-(tris((3-hydroxy-2,3-dimethylbutan-2-yl)oxy)-silyl)propyl) heptadecan-1-aminium chloride

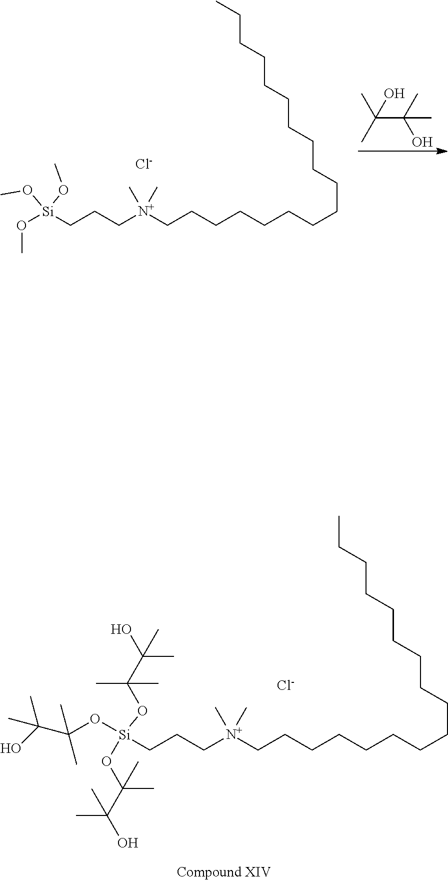

A 1 L round bottom reaction vessel was outfitted with a heating mantle, stir bar, downward condenser, receiving flask, oil bubbler, a thermoprobe for the pot, and a thermoprobe for the head. The reactor was charged with 59.7 mL of a 67% solution of dimethyloctadecyl[3-(trimethoxysilyl)propyl]ammonium chloride, 28.5 g of pinacol and ˜300 mL of DMF. The mixture was heated to 145° C. (pot temp) and the methanol was collected until the head temp drops and no more MeOH evolved. The reaction was then cooled and DMF evaporated at reduced pressure. Toluene (250 ml) was added, stirred at room temp for 1 hour, and then evaporated at reduced pressure. Acetonitrile (250 ml) was added, stirred at room temp for 1 hour, and then evaporated at reduced pressure. The resultant product was placed under high vac overnight to yield an off-white viscous liquid (55 g; 90% yield). The NMR spectrum conformed to the expected product.

### Example 3. Synthesis of Compound XV

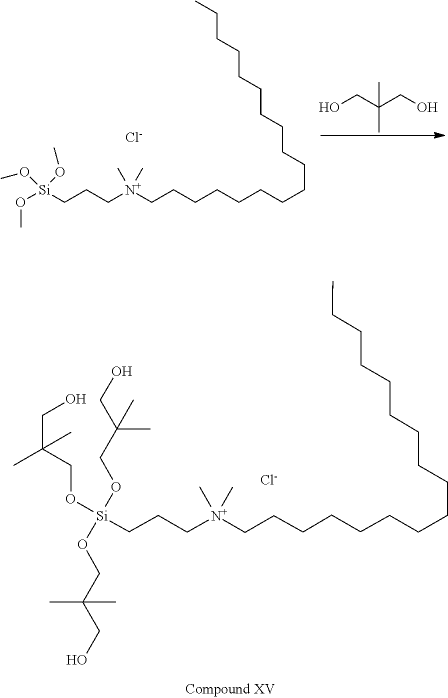

A 1 L round bottom reaction vessel was outfitted with a heating mantle, stir bar, downward condenser, receiving flask, oil bubbler, a thermoprobe for the pot, and a thermoprobe for the head. The reactor was charged with 59.7 mL of a 67% solution of dimethyloctadecyl[3-(trimethoxysilyl)propyl]ammonium chloride, 28.8 g of neopentylglycol and ˜300 mL of DMF. The mixture was heated to 145° C. (pot temp) and the methanol was collected until the head temp drops and no more MeOH evolved. The reaction was then cooled and DMF evaporated at reduced pressure. Toluene (250 ml) was added, stirred at room temp for 1 hour, and then evaporated at reduced pressure. Acetonitrile (250 ml) was added, stirred at room temp for 1 hour, and then evaporated at reduced pressure to give the desired compound (55 g; 96% yield). The NMR spectrum conformed to the expected product.

### Example 4. Dimethyl-octadecyl-[3-tris(2-carboxy-3-hydroxy-2-methyl-propoxy)silylpropyl]ammonium chloride

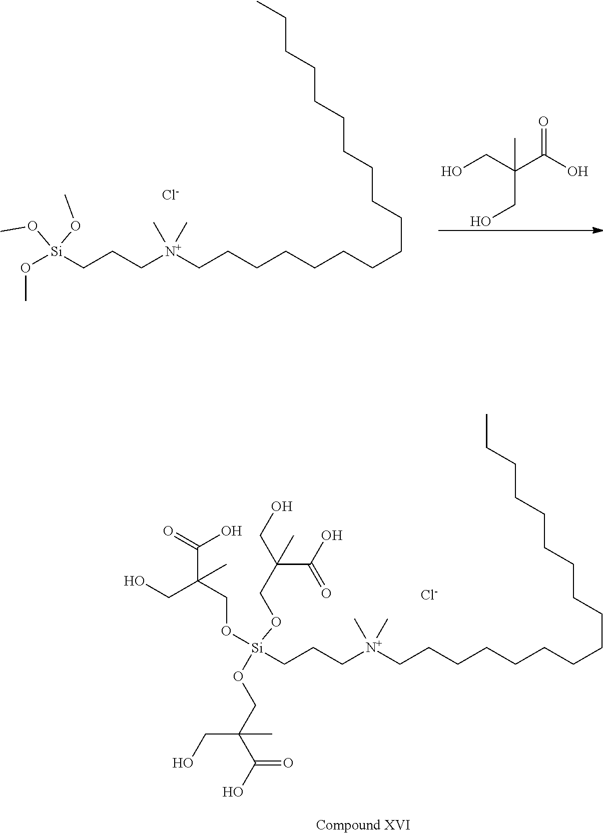

A 1 L round bottom reaction vessel was outfitted with a heating mantle, stir bar, downward condenser, receiving flask, oil bubbler, a thermoprobe for the pot, and a thermoprobe for the head. The reactor was charged with 59.7 mL of a 67% solution of dimethyloctadecyl[3-(trimethoxysilyl)propyl]ammonium chloride, 32.4 g of Bis (hydoxymethane)propanoic acid and ˜300 mL of DMF. The mixture was heated to 145° C. (pot temp) and the methanol was collected until the head temp drops and no more MeOH evolved. The reaction was then cooled and DMF was then evaporated at reduced pressure. Toluene (250 ml) was added, stirred at room temp for 1 hour, and then evaporated at reduced pressure. Acetonitrile (250 ml) was added, stirred at room temp for 1 hour, and then evaporated at reduced pressure. The resultant product was placed under high vac overnight to yield an off-white viscous semi-solid (62 g; 96% yield). The NMR was consistent with the proposed structure. The product dissolved readily in water and was stable for weeks.

### Example 5. Dimethyl-octadecyl-[3-[tris(3-hydroxypropoxy)silyl]propyl]ammonium chloride

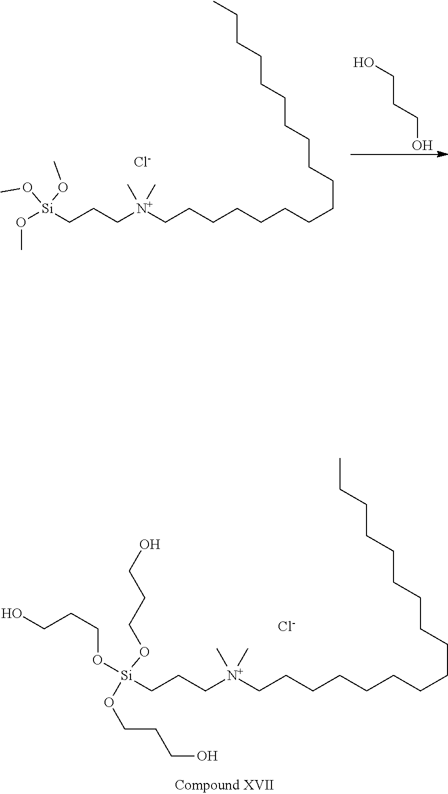 [SMILES: CCCCCCCCCCCCCCCCCC[N+](C)(C)CCC[Si](OC)(OC)OC ; [Cl-] ; OCCCO ; CCCCCCCCCCCCCCCCC[N+](C)(C)CCC[Si](OCCCO)(OCCCO)OCCCO ; [Cl-]]

A 1 L round bottom reaction vessel was outfitted with a heating mantle, stir bar, downward condenser, receiving flask, oil bubbler, a thermoprobe for the pot, and a thermoprobe for the head. The reactor was charged with 60 g of dimethyloctadecyl[3-(trimethoxysilyl)propyl]ammonium chloride, 28 g of 1,3-propanediol and ˜300 mL of DMF. The mixture was heated to 145° C. (pot temp) and the methanol was collected until the head temp drops and no more MeOH evolved. The reaction was then cooled and DMF evaporated at reduced pressure. Toluene (250 ml) was added, stirred at room temp for 1 hour, and then evaporated at reduced pressure. Acetonitrile (250 ml) was added, stirred at room temp for 1 hour, and then evaporated at reduced pressure. The resultant product was placed under high vac overnight to yield an off-white viscous liquid (74 g; 99% yield). The NMR was consistent with the proposed structure. The product dissolved readily in water and was stable for weeks.

### Example 6. Synthesis of Compound XVIII

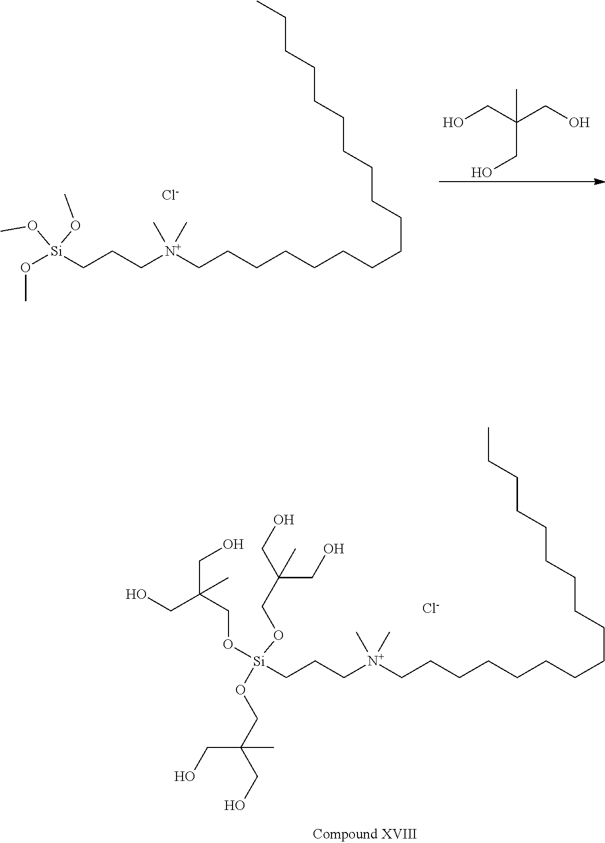

A 1 L round bottom reaction vessel was outfitted with a heating mantle, stir bar, downward condenser, receiving flask, oil bubbler, a thermoprobe for the pot, and a thermoprobe for the head. The reactor was charged with 59.7 mL of a 67% solution of dimethyloctadecyl[3-(trimethoxysilyl)propyl]ammonium chloride, 30 g of tris(hydroxymethyl) ethane and ˜300 mL of DMF. The mixture was heated to 145° C. (pot temp) and the methanol was collected until the head temp drops and no more MeOH evolved. The reaction was then cooled and DMF evaporated at reduced pressure. Toluene (250 ml) was added, stirred at room temp for 1 hour, and then evaporated at reduced pressure. Acetonitrile (25 0 ml) was added, stirred at room temp for 1 hour, and then evaporated at reduced pressure. The resultant product was placed under high vac overnight to yield an off-white viscous liquid (60 g; 98% yield). The NMR spectrum conformed to the expected product.

### Example 7. 2-[[1-[[3-[Dimethyl(octadecyl)ammonio]propyl-[3-hydroxy-2-(hydroxymethyl)-2-(2-sulfoethylamino)propoxy]-[3-hydroxy-2-(hydroxymethyl)-2-(2-sulfoethylamino)propoxy]silyl]oxymethyl]-2-hydroxy-1-(hydroxymethyl)ethyl]amino]-ethanesulfonate

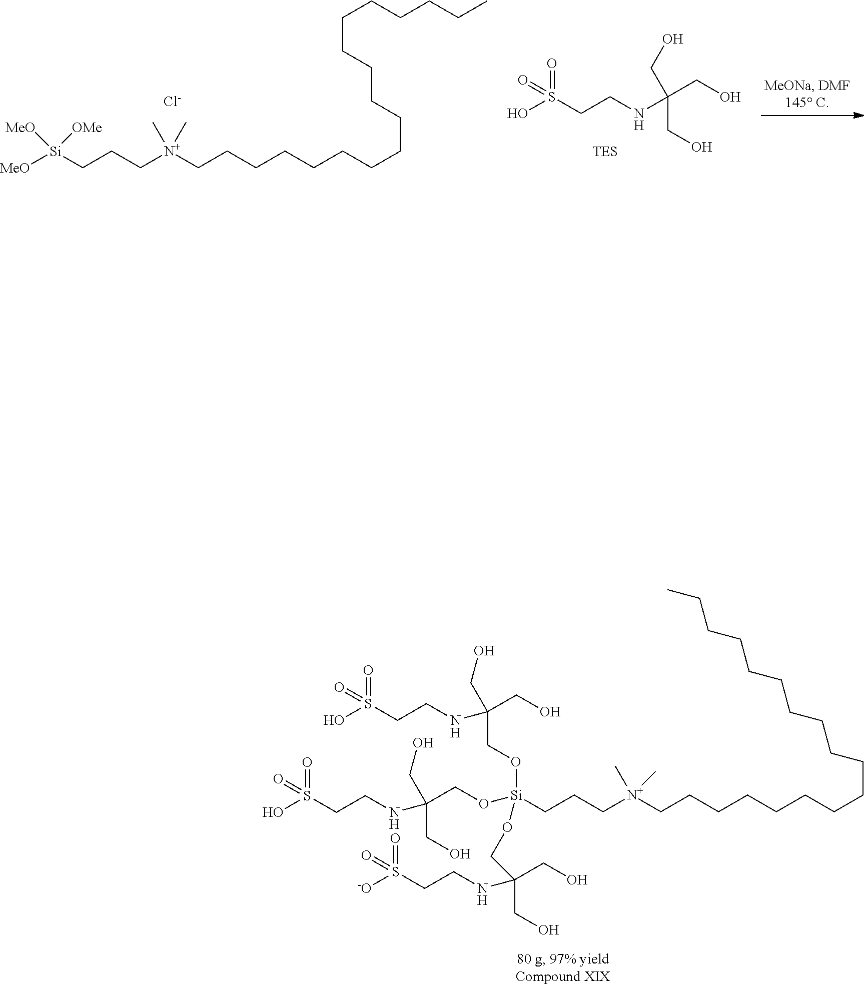

A 500 mL round bottom reaction vessel was outfitted with an oil bath, stir bar, distillation head and condenser, receiving flask, oil bubbler, gas inlet that allows argon to pass through vessel and distillation setup, an immersion thermoprobe for the oil bath, and a thermoprobe for the head. The reactor is charged with 47.6 mL of a 42% methanolic solution of dimethyloctadecyl[3-(trimethoxysilyl)propyl]ammonium chloride (Aldrich), 200 mL DMF, 27.7 g (3 equiv.) of N-Tris(hydroxymethyl)methyl-2-aminoethanesulfonic acid (TES) and 8.7 mL of 25% MeONa in MeOH. The mixture is heated to 125 degrees C. (oil temp) and the methanol is collected by distillation. As the reaction is heated a homogenous solution is formed. As the methanol is distilled off a white precipitate begins to form. The reaction is heated until the head temp drops and no more MeOH is evolved (about 1.5 hours). The reaction is then cooled to room temperature and the precipitate (NaCl) is removed by filtration through a glass fiber filter and the DMF was concentrated to about 100 mL total volume at reduced pressure. The solution is cooled to rt. A 1 L flask under argon and a stir bar is charged with anhydrous acetonitrile (1000 mL) and cooled to ˜10 C. The DMF solution is added slowly to rapidly stirring acetonitrile to form a white suspension. Stirring is continued for 30 minutes. The colorless solid is collected on a glass frit under argon and washed with acetonitrile and then ether. The hygroscopic solid is then transferred to a vacuum flask and dried under vacuum. The resulting fine powder dissolves quickly in water with gentle heating. Initially it is hazy but slowly clears. A 1% solution in water remains clear for >5 months.

### Example 8. 2-[[1-[[3-[Dimethyl(octadecyl)ammonio]propyl-[3-hydroxy-2-(hydroxymethyl)-2-(2-sulfoethylamino)propoxy]-[3-hydroxy-2-(hydroxymethyl)-2-(2-sulfoethylamino)propoxy]silyl]oxymethyl]-2-hydroxy-1-(hydroxymethyl)ethyl]amino]-ethanesulfonate

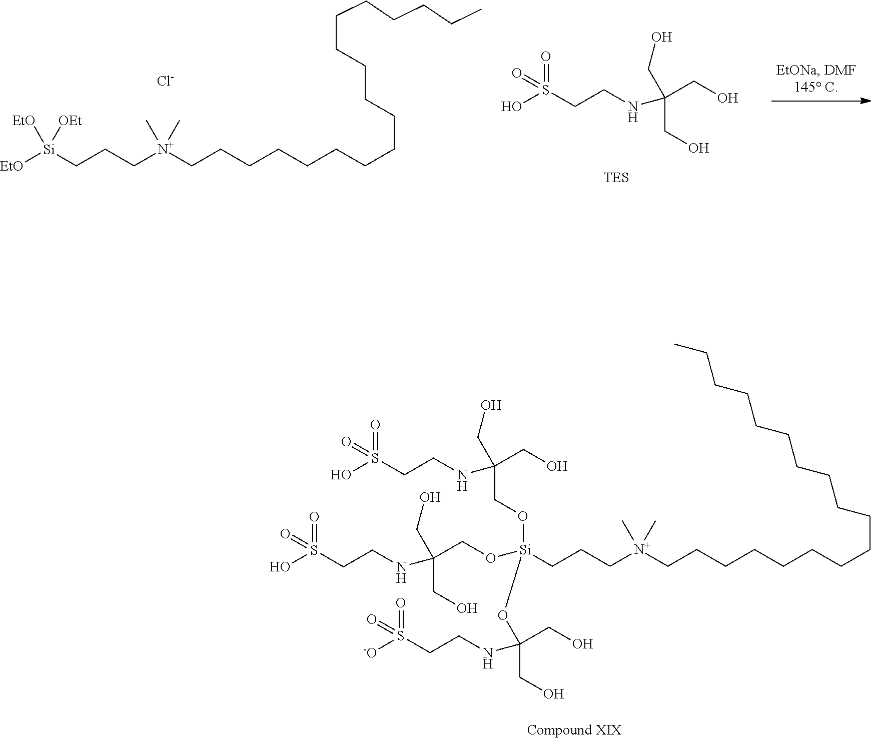

A 500 mL round bottom reaction vessel was outfitted with an oil bath, stir bar, distillation head and condenser, receiving flask, oil bubbler, gas inlet that allows argon to pass through vessel and distillation setup, an immersion thermoprobe for the oil bath, and a thermoprobe for the head. The reactor is charged with 47.6 mL of a 42% methanolic solution of dimethyloctadecyl[3-(trimethoxysilyl)propyl]ammonium chloride (Aldrich), 200 mL DMF, 27.7 g (3 equiv.) of N-Tris(hydroxymethyl)methyl-2-aminoethanesulfonic acid (TES) and 8.7 mL of 25% EtONa in EtOH. The mixture is heated to 125 degrees C. (oil temp) and the methanol is collected by distillation. As the reaction is heated a homogenous solution is formed. As the methanol is distilled off a white precipitate begins to form. The reaction is heated until the head temp drops and no more MeOH is evolved (about 1.5 hours). The reaction is then cooled to room temperature and the precipitate (NaCl) is removed by filtration through a glass fiber filter and the DMF was concentrated to about 100 mL total volume at reduced pressure. The solution is cooled to rt. A 1 L flask under argon and a stir bar is charged with anhydrous acetonitrile (1000 mL) and cooled to ˜10 C. The DMF solution is added slowly to rapidly stirring acetonitrile to form a white suspension. Stirring is continued for 30 minutes. The colorless solid is collected on a glass frit under argon and washed with acetonitrile and then ether and dried under vacuum.

### Example 9. 3,3′-(((2-Amino-3-hydroxy-2-(hydroxymethyl)propoxy) (3-(dimethyl(octadecyl)ammonio)propyl)silanediyl)bis(oxy)) bis(1-hydroxy-2-(hydroxymethyl)propan-2-aminium)methanesulfonate

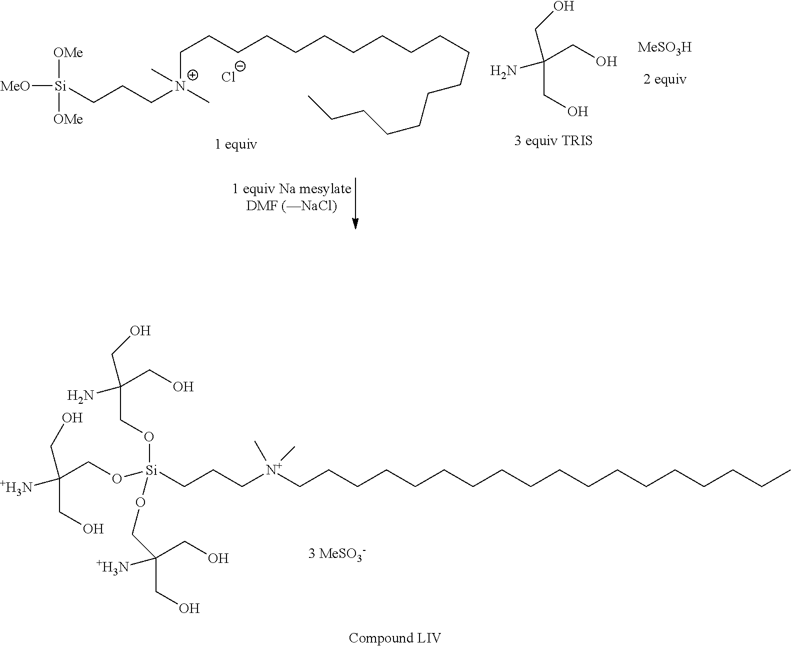 [SMILES: "MeSO3H" ; CCCCCCCCCCCCCCCCCC[N+](C)(C)CCC[Si](OC)(OC)OC ; [Cl-] ; NC(CO)(CO)CO ; CCCCCCCCCCCCCCCCCC[N+](C)(C)CCC[Si](OCC(N)(CO)CO)(OCC([NH3+])(CO)CO)OCC([NH3+])(CO)CO]

Experiment 1. A 500 mL round bottom reaction vessel is outfitted with an oil bath, stir bar, distillation head and condenser, receiving flask, oil bubbler, gas inlet that allows argon to pass through vessel and distillation setup, an immersion thermoprobe for the oil bath, and a thermoprobe for the head. The reactor is charged with 47.6 mL of a 42% solution of dimethyloctadecyl[3-(trimethoxysilyl)propyl]ammonium chloride (Aldrich), 200 mL DMF, 3 equiv. of TRIS, 2 equiv. of MeSO₃H, and 1 equiv. of Na mesylate.

The mixture is heated to 125° C. (oil temp) and the methanol is collected by distillation. At this stage the precipitate (NaCl) is removed by filtration and the DMF is concentrated to about 100 mL total volume at reduced pressure. After cooling the solution to rt, a 1 L flask under argon and a stir bar is charged with anhydrous acetonitrile and cooled to ˜10 C. The DMF solution is added slowly to rapidly stirring acetonitrile (1000 mL) to generate a white suspension of the product.

Experiment 2. A 500 ml round bottom reaction vessel is outfitted with an oil bath, stir bar, distillation head and condenser, receiving flask, oil bubbler, gas inlet that allows nitrogen to pass through vessel and distillation setup, an immersion thermoprobe for the oil bath, and a thermoprobe for the head. The reactor is charged with 300 ml DMF, 21.9 g TRIS (3 equiv.), 7.13 g (1 equiv.) sodium mesylate, 11.6 g (2 equiv.) methanesulfonic acid, and then 71.43 ml of a 42% solution of dimethyloctadecyl[3-(trimethoxysilyl)propyl]ammonium chloride (Aldrich). The mixture is heated to 80 C (oil temp) for 2 h and then heated to 120° C. and the methanol is collected by distillation. As the reaction is heated, a homogenous solution forms. As the methanol is distilled off a white precipitate begins to form. The reaction is heated until the head temperature drops and no more MeOH is evolved (about 1.5 hours). Nitrogen is then allowed to sweep through the vessel to remove MeOH and then cooled to about 80° C. Vacuum is carefully applied to distill all MeOH possible. Precipitated NaCl is then removed by centrifugation and decanting. A 1 L flask under argon and a stir bar is charged with anhydrous acetonitrile and cooled to ˜10° C. The DMF solution is added slowly to rapidly stirring acetonitrile to form a white suspension. Stirring is continued for 30 minutes. The colorless solid is collected on a glass frit under argon and washed with acetonitrile and then ether. The hygroscopic solid is then transferred to a vacuum flask and dried under vacuum. The yield is 50 g (81%).

### Example 10: Hydrolysis of Quaternary Ammonium Silane Compound VII

In this example, hydrolysis of Compound VII was evaluated in the mass-spectrometry study (Direct Infusion Mass Spectrometry, ESI+ mode).

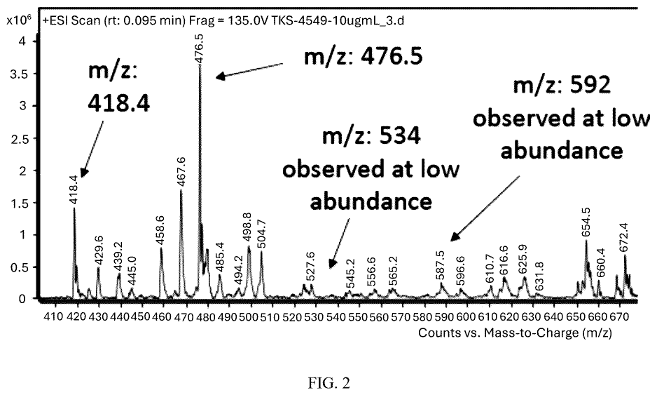

In a first experiment, Compound VII was dissolved in propylene glycol and diluted with acetonitrile to a final concentration of 10 μg/mL in the sample. The mass spectrum of the sample is shown in FIG. 2. The mass-spectrum contains a major peak with m/z of 476.5 corresponding to N-(3-(dihydroxy (2-hydroxypropoxy) silyl)propyl)-N,N-dimethyloctadecan-1-aminium cation formed as a result of partial hydrolysis of Compound VII where only two side chains of Compound VII were hydrolyzed, and a smaller peak with m/z of 418.4 corresponding to N,N-dimethyl-N-(3-(trihydroxysilyl)propyl)octadecan-1-aminium cation formed as a result of complete hydrolysis of Compound VII. Two minor peaks with m/z of 592 (Compound VII) and m/z of 534 (mono-hydrolyzed species of Compound VII) are present at low abundance. Partial hydrolysis of Compound VII when dissolved in acetonitrile is due to the required use of 0.1% aqueous formic acid in the mobile phase for the HPLC-mass spec analysis. Exposure time during the HPLC-mass spec analysis is less than 45 seconds, further confirming the rapid hydrolysis of the quaternary ammonium silane upon exposure to water.

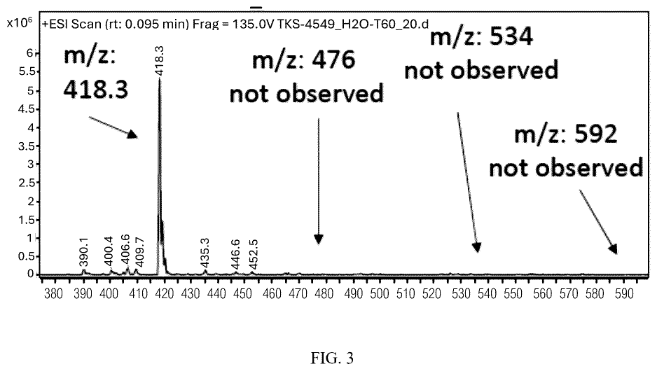

In a second experiment, Compound VII was diluted in water to a final concentration of 10 μg/mL in the sample. The mass spectrum of the sample was recorded at a 30-minute time point and is shown FIG. 3. Compound VII was completely hydrolyzed within the 30-minute time frame according to the following reaction scheme:

This data suggests that full hydrolysis of Compound VII occurred in aqueous medium within a 30 to 60 minutes time frame.

Additionally, hydrolysis of Compound VII formulated with inactive ingredients was evaluated in the mass-spectrometry study (Direct Infusion Mass Spectrometry, ESI+mode).

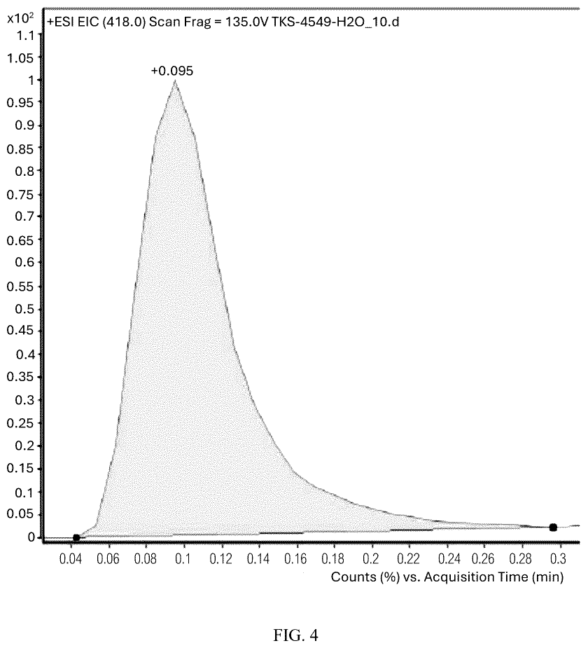

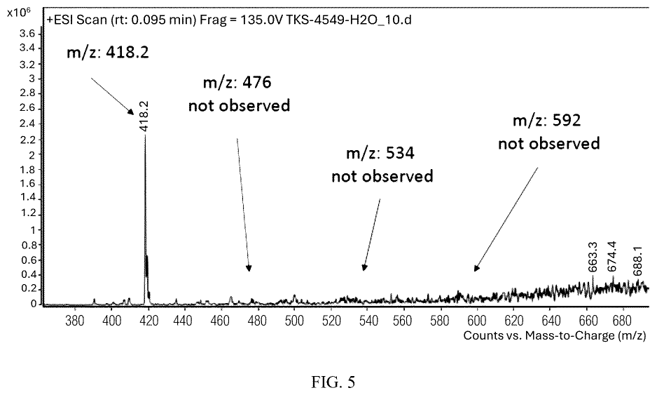

0.50% of Compound VII was formulated with 2% propylene glycol, 1% HPMC, 0.1% poloxamer 407, 2% PEG, and 94.40% water. The mass spectrum of the sample is shown in FIG. 5. The mass-spectrum contains one major peak with m/z of 418.4 corresponding to N,N-dimethyl-N-(3-(trihydroxysilyl)propyl)octadecan-1-aminium cation formed as a result of complete hydrolysis of Compound VII. FIG. 4 shows an extracted ion chromatogram of the cation with m/z of 418.

### Example 11 Preparation of an Quaternary Ammonium Silane Hydrogel Formulation

180 mL of distilled water was added to a 250 mL screw-top media bottle with a stir bar. 2 g of HPMC having a viscosity 2600-5600 in H₂O (20° C.); was added slowly under vigorous stirring. The mixture was then stirred overnight until the HPMC was fully dissolved and the mixture was homogenous and clear. 4 g of PEG Mn 6000 and 0.2 g of Poloxamer 407 was added with vigorous stirring until homogenous and clear. 5.26 g of 19% Compound XIII in propylene glycol was slowly added via syringe while stirring. The Compound XIII solution was dispersed as quickly as possible by rolling or shaking the bottle. The viscosity of the solution was determined to be 1728 mPa·S. The media bottle was then sealed and the mixture was heated in an autoclave to 123° C. for 40 minutes after which it was cooled to 40° C., removed from the autoclave, and cooled to room temp with stirring. The pH was then determined and adjusted to about a pH of 6.0 using 0.1M methanesulphonic acid, and the solution was brought up to 200 mL by the addition of distilled water. The final viscosity was determined to be about 1250 cP as measured using rotational viscometry. At the time of this filing, the quaternary ammonium silane aqueous hydrogel has remained clear and stable for over one year.

### Example 12 Preparation of an Quaternary Ammonium Silane Hydrogel Formulation

160 mls of distilled water was added to a 250 mL screw-top media bottle. 4 g of HPMC having a viscosity 2600-5600 in H₂O (20° C.) was added slowly under vigorous stirring. The mixture was stirred overnight and the HPMC was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, and the final volume was adjusted to 200 ml. The media bottle was sealed and the mixture was heated in an autoclave to 123° C. for 20 minutes after which it was cooled to 40° C., removed from the autoclave, and cooled to room temperature with stirring. Hydrolysis of Compound XIX produces 1 g of dimethyloctadecyl[3-(trihydroxysilyl)propyl]ammonium and 0.92 g of TES, giving a final concentration of 0.5% and 0.46%, respectively. The final viscosity was determined to be about 1600 cP as measured using rotational viscometry. At the time of this filing, the quaternary ammonium silane aqueous hydrogel has remained clear and stable for over one year.

### Example 13: Hydrogels Containing Ionic or Non-Ionic, Carboxylic Acid Group Containing Ingredients

160 mls of distilled water was added to a 250 mL screw-top media bottle. 4 g of Xanthan gum was added slowly under vigorous stirring. The mixture was stirred overnight and the Xanthan gum was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, resulting in the immediate precipitation of Compound XIX. At this point the process was stopped.

160 mls of distilled water was added to a 250 mL screw-top media bottle. 0.2 g of carbomer copolymer A was added slowly under vigorous stirring. The mixture was stirred overnight and the carbomer copolymer A was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, resulting in the immediate precipitation of Compound XIX. At this point the process was stopped.

160 mls of distilled water was added to a 250 mL screw-top media bottle. 0.2 g of carbomer copolymer B was added slowly under vigorous stirring. The mixture was stirred overnight and the carbomer copolymer B was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, resulting in the immediate precipitation of Compound XIX. At this point the process was stopped.

160 mls of distilled water was added to a 250 mL screw-top media bottle. 0.2 g of carrageenan was added slowly under vigorous stirring. The mixture was stirred overnight and the carrageenan was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, resulting in the immediate precipitation of Compound XIX. At this point the process was stopped.

160 mls of distilled water was added to a 250 mL screw-top media bottle. 0.2 g of carboxymethylcellulose Sodium was added slowly under vigorous stirring. The mixture was stirred overnight and the Carboxymethylcellulose Sodium was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, resulting in the immediate precipitation of Compound XIX. At this point the process was stopped.

160 mls of distilled water was added to a 250 mL screw-top media bottle. 0.2 g of Sodium Alginate was added slowly under vigorous stirring. The mixture was stirred overnight and the Sodium Alginate was fully dissolved, with the mixture homogenous and clear. Zinc gluconate (0.2 g), sorbitol (10 g), glycerin (4 g), and Poloxamer 407 (0.4 g) were added to the mixture and stirred until homogenous and clear. 1.92 g of Compound XIX was added while stirring, resulting in the immediate precipitation of Compound XIX. At this point the process was stopped.

160 mls of distilled water was added to a 250 mL screw-top media bottle. 4 g of HPMC having a viscosity 2600-5600 in H₂O (20° C.) was added slowly under vigorous stirring. The mixture was stirred overnight and the HPMC was fully dissolved, with the mixture homogenous and clear. Zinc gluconate (0.2 g), sorbitol (10 g), glycerin (4 g), and sodium hyaluronate (0.4 g) were added to the mixture and stirred until homogenous and clear. 1.92 g of Compound XIX was added while stirring, resulting in the immediate precipitation of Compound XIX. At this point the process was stopped.

### Example 14 In Vivo Testing of Quaternary Ammonium Silane Hydrogel Formulation to Treat Canis lupus familiaris Periodontitis and Gingivitis

The goal of this study was to evaluate the effectiveness of a single application of a quaternary ammonium silane hydrogel formulation as described herein in treating Canis lupus familiaris periodontitis and gingivitis.

Periodontal diseases, including gingivitis and chronic periodontitis, are common conditions in human and dogs and, left untreated, can lead to tooth loss. The prevalence of periodontal disease in dogs reaches up to 80-85% over age 2. The initial stage of disease is gingivitis caused by the formation of plaque on the tooth surface and a subsequent host inflammatory response. Unless the plaque is removed, gingivitis may progress to periodontitis resulting in irreversible tissue destruction and eventual destruction of the periodontal ligament, gum recession and the breakdown of supporting tissues leading to loss of teeth. Davis, I. J., et al., A cross-sectional survey of bacterial species in plaque from client owned dogs with healthy gingiva, gingivitis or mild periodontitis. PLOS One, 2013. 8 (12): p. e83158.1.

### Study Design Overview

For each enrolled dog, Next-Generation Sequencing, NGS (DNA sequencing,) was used to initially identify, quantify, and define microbes that inhabit the wounds' biofilm communities. NGS was done pretreatment and again one month following treatment. The hydrogel was applied topically directly to the gingiva immediately following the standard of care (SOC) for treating plaque (general anesthesia, surgical descaling, tooth cleaning and polishing=SOC.) Enrollment was fulfilled beyond the initial target of eighty dogs (75 dogs plus controls). The study and protocol allow for ease of extension and addition of comparative groups. Data from NGS has resulted in identification of microbes in the dysbiotic biofilm of the gingival sulcus (many hundreds sorted by percentage of population) and one month later the changes brought about by treatment. It is generally accepted that dysbiosis (an imbalance in the types of organism in an animals' microflora) leads to gingivitis. Each of the dogs has a clinical veterinary chart with recorded history and known owner. The breed and sex of each dog is known.

### Study Design Protocol

1. Patient exam with staging of dental/periodontal disease.
2. Inform client of study, obtain consent form and schedule procedure.
3 Obtain baseline bloodwork.
4. Follow standard anesthesia protocol.
5. Obtain full mouth pictures prior to cleaning.
6. Swab periodontal areas of mouth with two separate sterile swabs.
7. Place swabs in sterile transfer tubes and label with patient information and date.
a. Send one swab to RTL Genomics for NSG sequencing.
b Send second swab to Biofilm Research Institute, LLC.
8. Complete dental prophy with periodontal scaling.
9 Apply ammonium silane hydrogel at room temperature to periodontal area of both upper and lower dental arcade (lingual and buccal sides).
10. Wake animal up and recover from anesthesia.
11. Schedule follow up swab for 4 weeks.
12. Return patient visit in 4 weeks.
13. Obtain two sample swabs of periodontal region.
14. Label tubes with appropriate patient data and dates.
15. Place swabs in sterile transfer tubes and label with patient information and date.
a. Send one swab to RTL Genomics for sequencing.
b. Send second swab to Biofilm Research Institute, LLC.

### Quaternary Ammonium Silane Aqueous Hydrogel Formulation

An animal oral wash hydrogel formulation was prepared having the following ingredients:

| | Canine Wash |
|---|---|
| Active | Ion I - 0.5%, derived from |
| | complete hydrolysis of |
| | Compound XIX |
| Gelling agent | HPMC 2% having a viscosity |
| | 2600-5600 in H₂O (20° C) |
| Adhesion agent | Poloxamer 407 0.20% |
| Wetting agent | Glycerin 2% |
| Aqueous solution | To 100% |
| Other | Zinc gluconate 0.1% |
| Buffer | methanesulfonic |
| | acid (<0.01%) |
| pH | 6.0 |
| cP | 1600 |

### Results

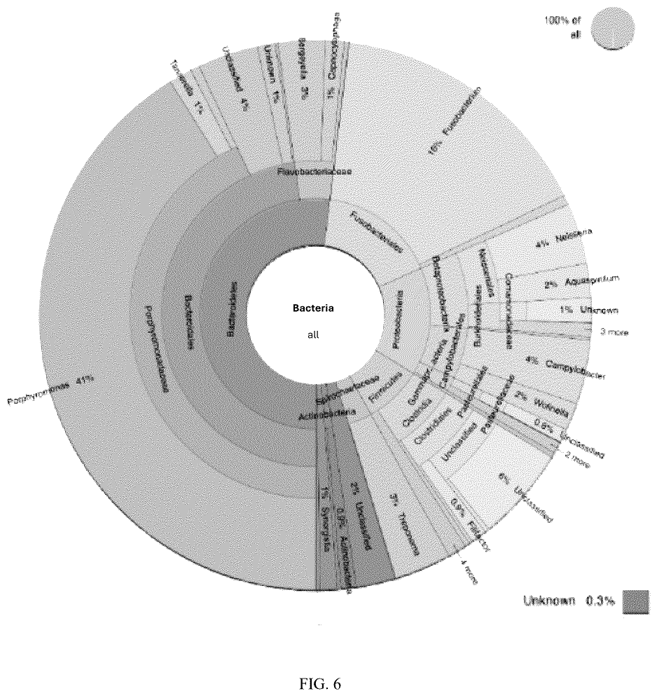

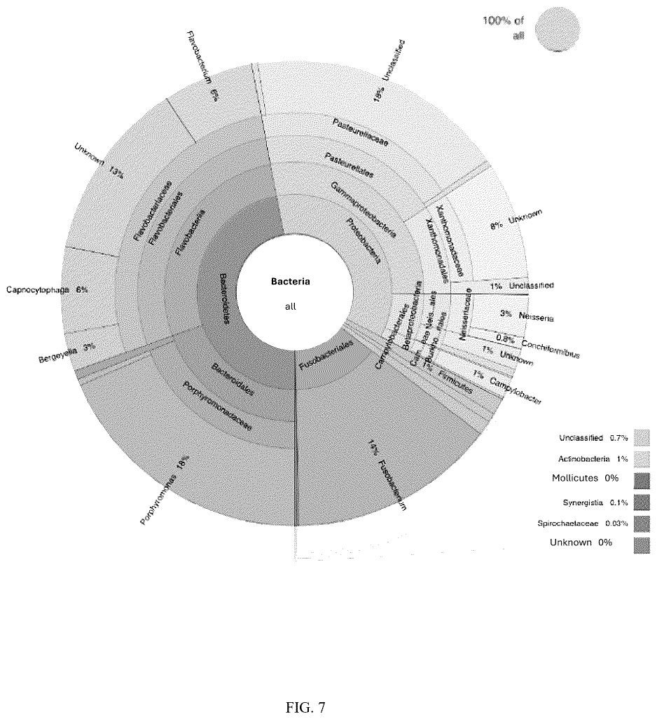

The data demonstrate that after applying a single application of the quaternary ammonium silane hydrogel, the pilot dog presented with a homeostatic microbiome and healthy gums for seven months (FIGS. 6-7). This sentinel animal indicated that a single application of ammonium silane hydrogel may provide long-term control of gingivitis/periodontitis.

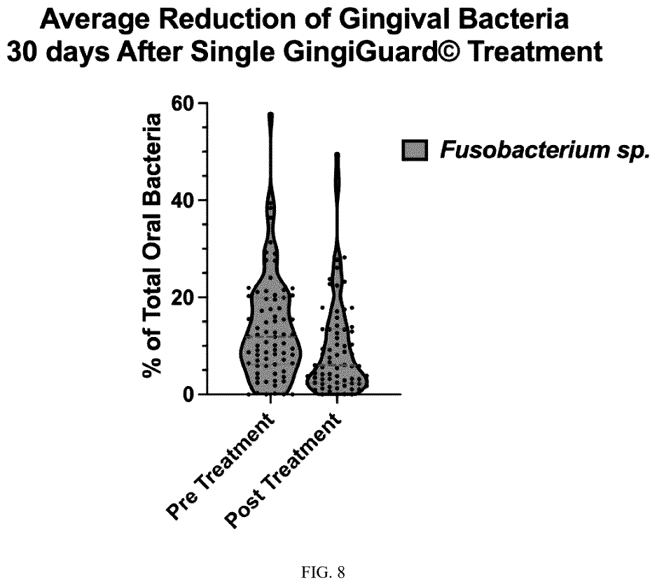

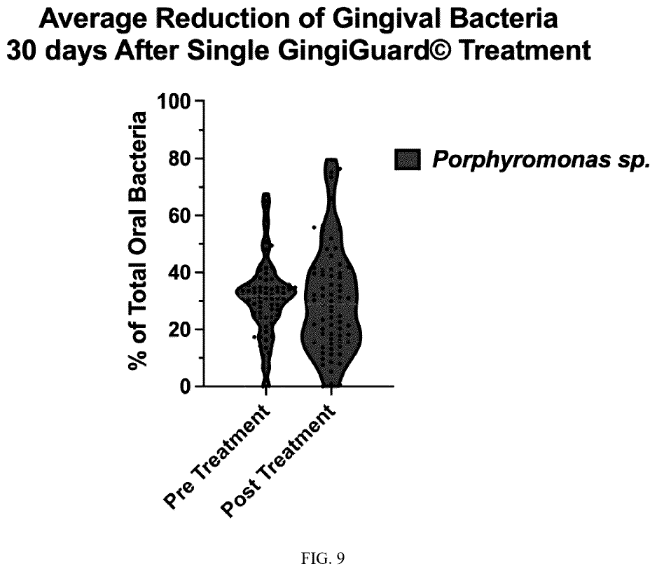

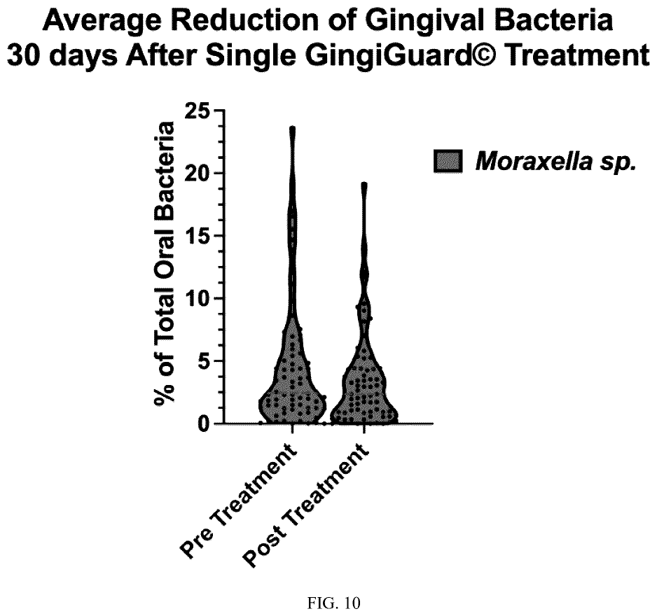

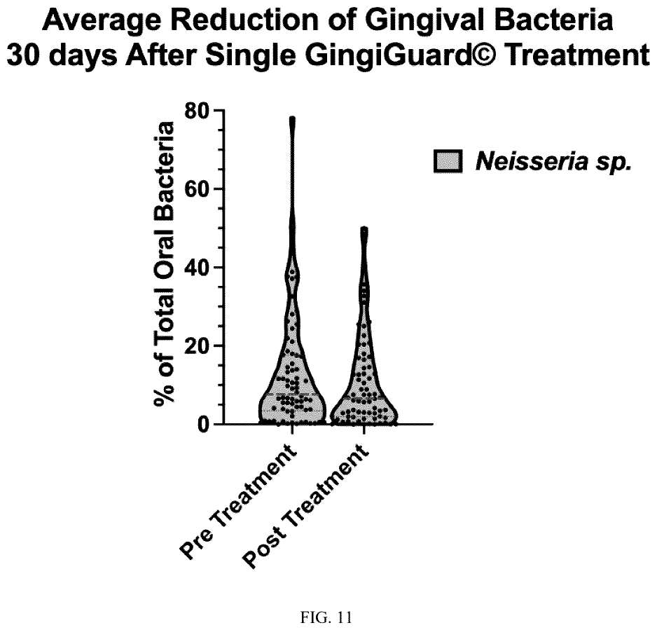

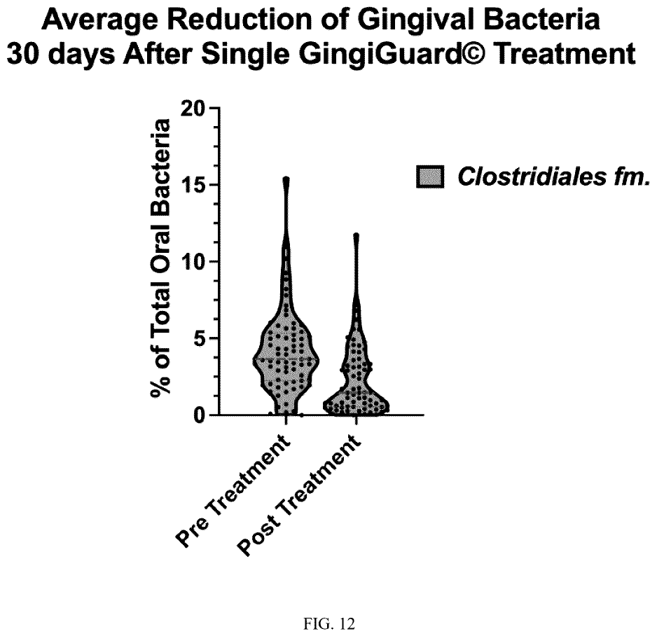

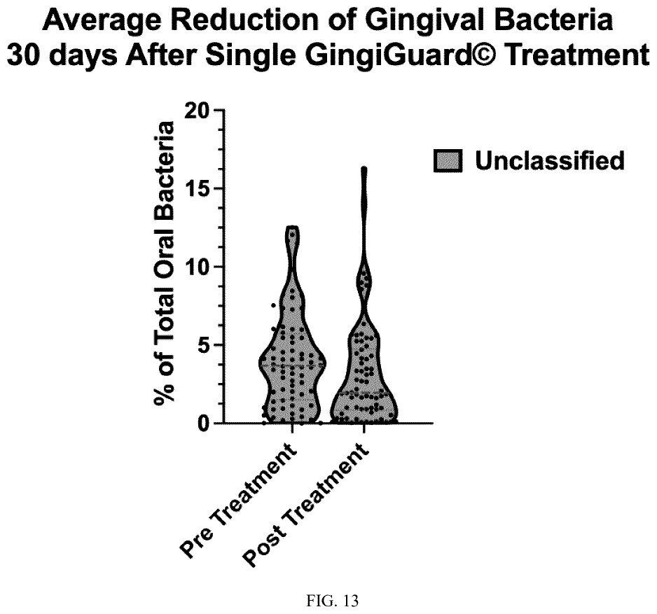

It is believed that the dysbiosis of the altered oral microbiome community may lead to periodontal disease. Porphyromonas and Fusobacterium, among other sub-dominant microbial populations, are considered contributing organisms to disease. As observed in FIGS. 8-13, several bacterial populations were present in decreased prevalence one month after a single application of the ammonium silane hydrogel.

Collectively, these data demonstrate the ability of the novel quaternary ammonium silane hydrogel to restore the microbiome populations to homeostasis after a single application for at least 30 days.

1. An aqueous hydrogel formulation comprising:
a) a quaternary ammonium silane compound selected from the group consisting of Formula A-D and Compounds I-XXV;
b) a non-ionic gelling agent;
c) a non-ionic adhesion agent;
d) a non-ionic wetting agent;
e) optionally a non-ionic osmotic agent; and
f) an aqueous solution; 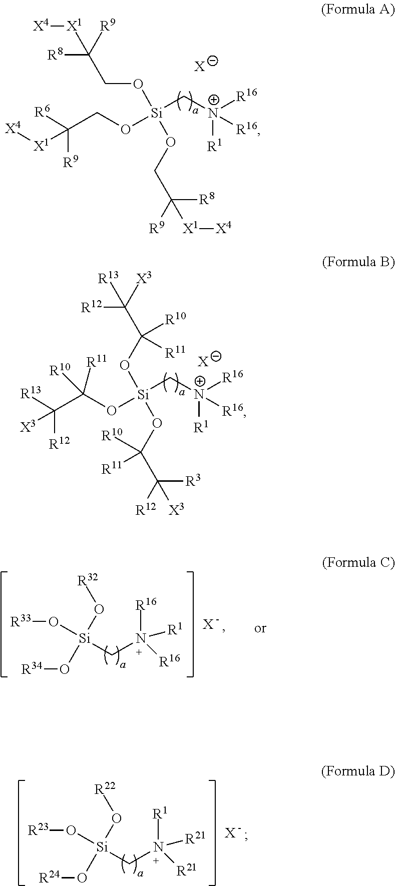
wherein:
a is 1, 2, 3, or 4;
R¹ is independently at each occurrence selected from the group consisting of C₆-C₂₂ alkyl and C₆-C₂₂ alkanoyl;
R⁸ is H or CH₂OH;
R⁹ is H or C₁-C₈ alkyl;
X³ is OH or CH₂OR⁷;
R¹⁰, R¹¹, and R¹² are independently at each occurrence selected from the group consisting of hydrogen, hydroxyl, CH₂OR⁷, CON(R⁷)₂, COOR⁷, C(O)R⁷, C₁-C₈ alkyl, C₁-C₈ hydroxyalkyl, and C₁-C₈ alkanoyl;
R¹³ is selected from the group consisting of hydroxyl, CH₂OR⁷, CON(R⁷)₂, COOR⁷, C(O)R⁷, C₁-C₈ alkyl, C₁-C₈ hydroxyalkyl, and C₁-C₈ alkanoyl;
R⁷ is independently at each occurrence selected from hydrogen, C₁-C₈ alkyl, C₁-C₈ hydroxyalkyl, and C₁-C₈ alkanoyl;
X¹ is NR¹⁷, CH₂, or C(O);
X⁴ is selected from the group consisting of hydroxyl, CH₂OR⁷, CON(R⁷)₂, COOR⁷, C(O)R⁷, C₁-C₈ hydroxyalkyl, and C₁-C₈ alkanoyl;
R¹⁶ is C₁-C₄ alkyl;
R¹⁷ is hydrogen, C₁-C₈ hydroxyalkyl, or C₁-C₈ alkanoyl;
X⁻ is an anion or is absent if the quaternary amine is balanced with an internal anion;
R³², R³³, R³⁴, R²², R²³, and R²⁴ are independently at each occurrence selected from the group consisting of: 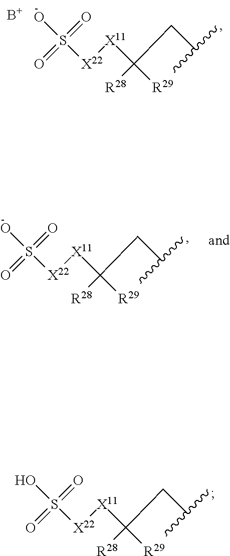
each R²¹ is independently selected from C₁-C₂₂alkyl and C₂-C₂₂ alkanoyl;
R²⁸ and R²⁹ are independently at each occurrence selected from hydrogen, halogen, hydroxyl, N (R⁷)₂, CH₂OR⁷, CON(R⁷)₂, COOR⁷, C(O)R⁷, C₁-C₈ alkyl, C₁-C₈ hydroxyalkyl, and C₁-C₈ alkanoyl;
X¹¹ is NR¹⁷, CH₂, CHOH, or C(O);
X²² is C₁-C₈ alkyl or C₁-C₃hydroxyalkyl;
B⁺ is a cation; and
wherein Compounds I-XXV are:

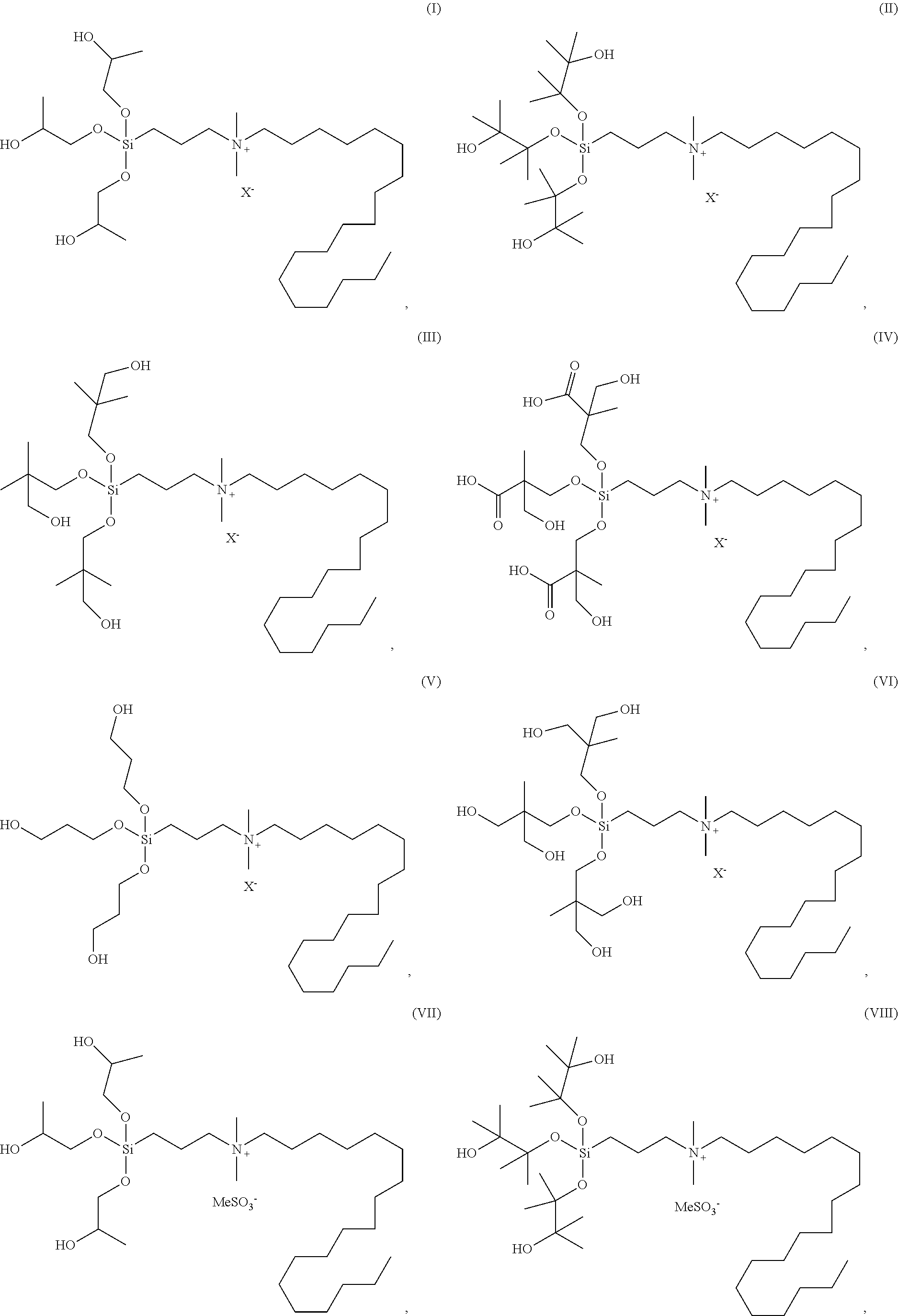

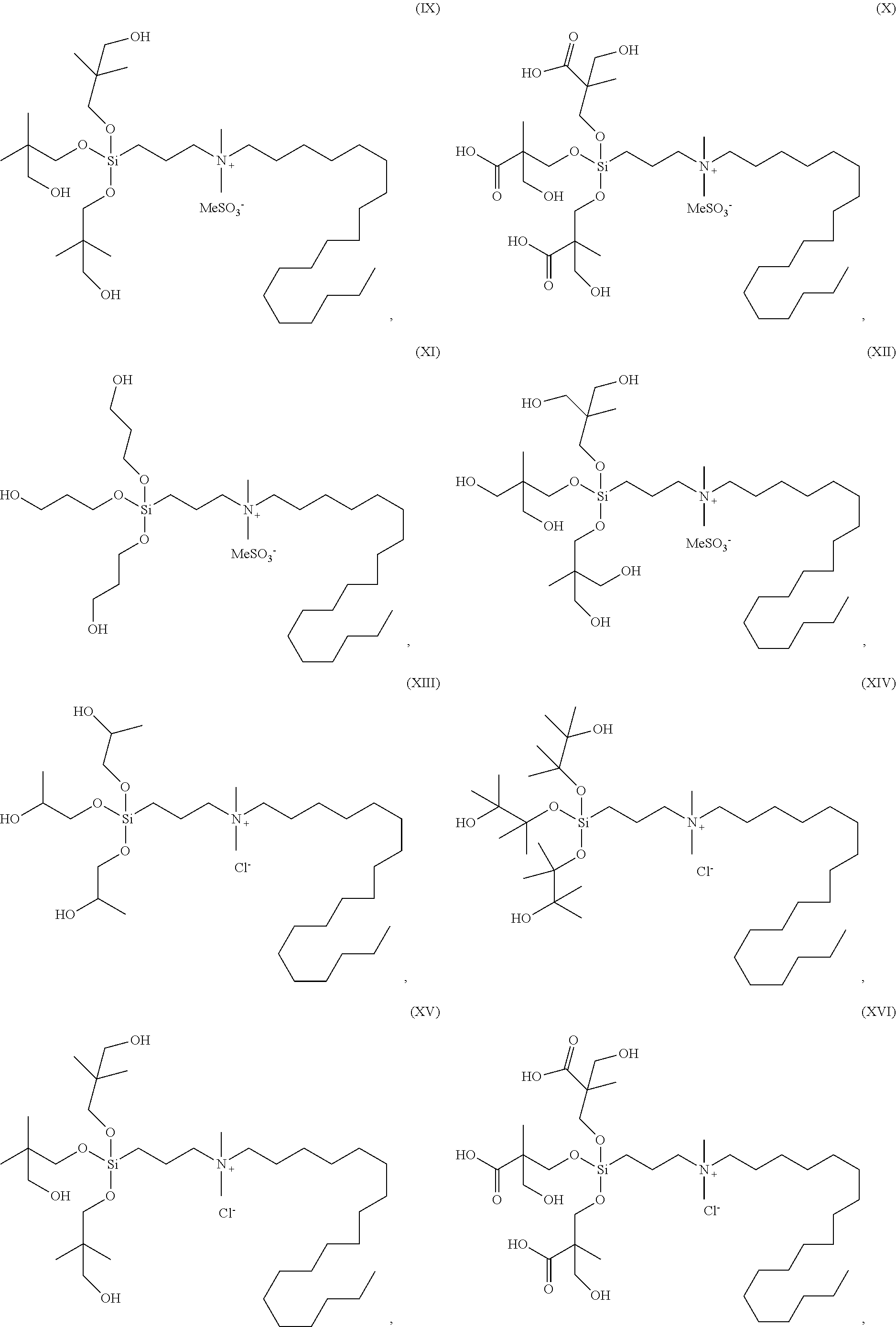

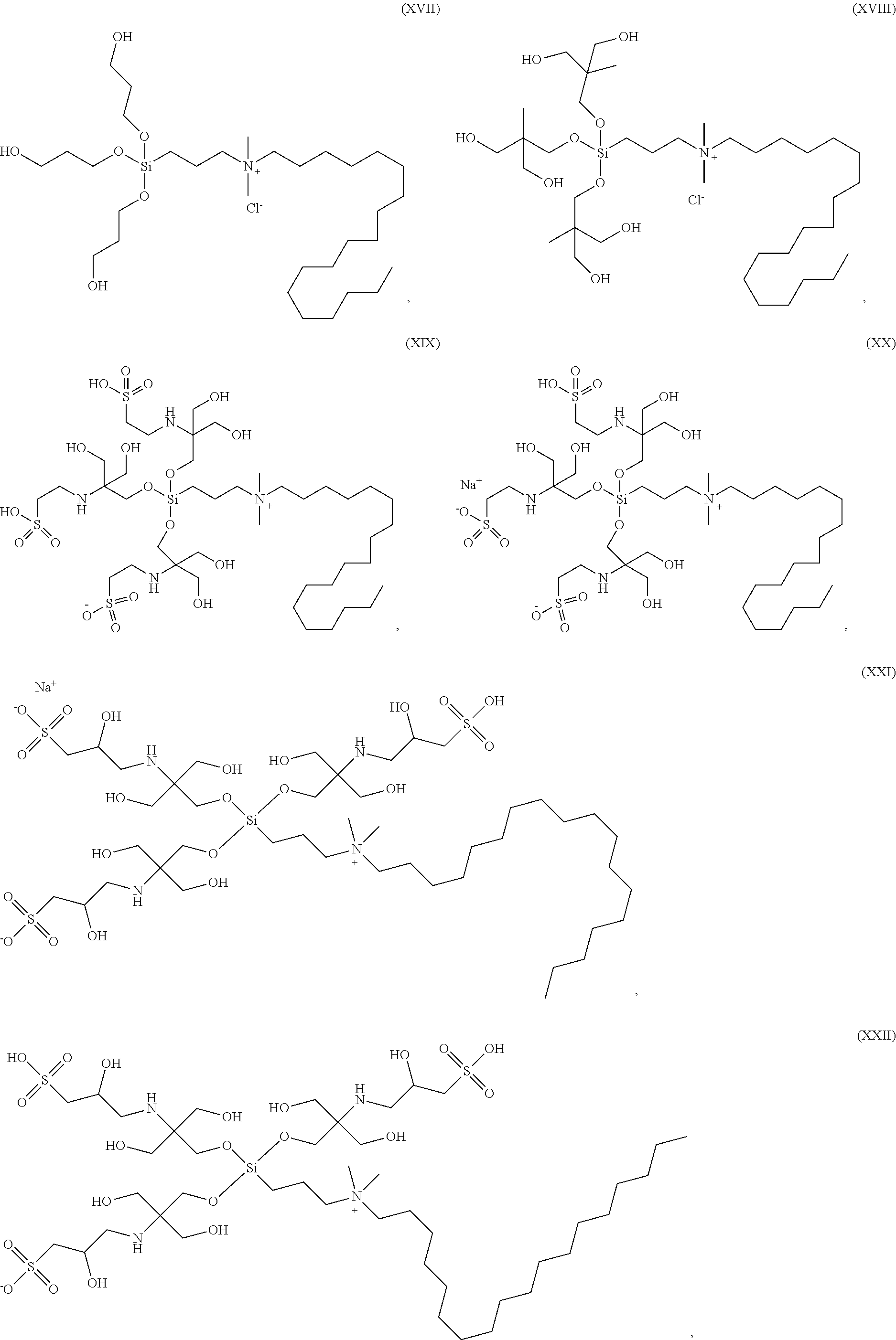

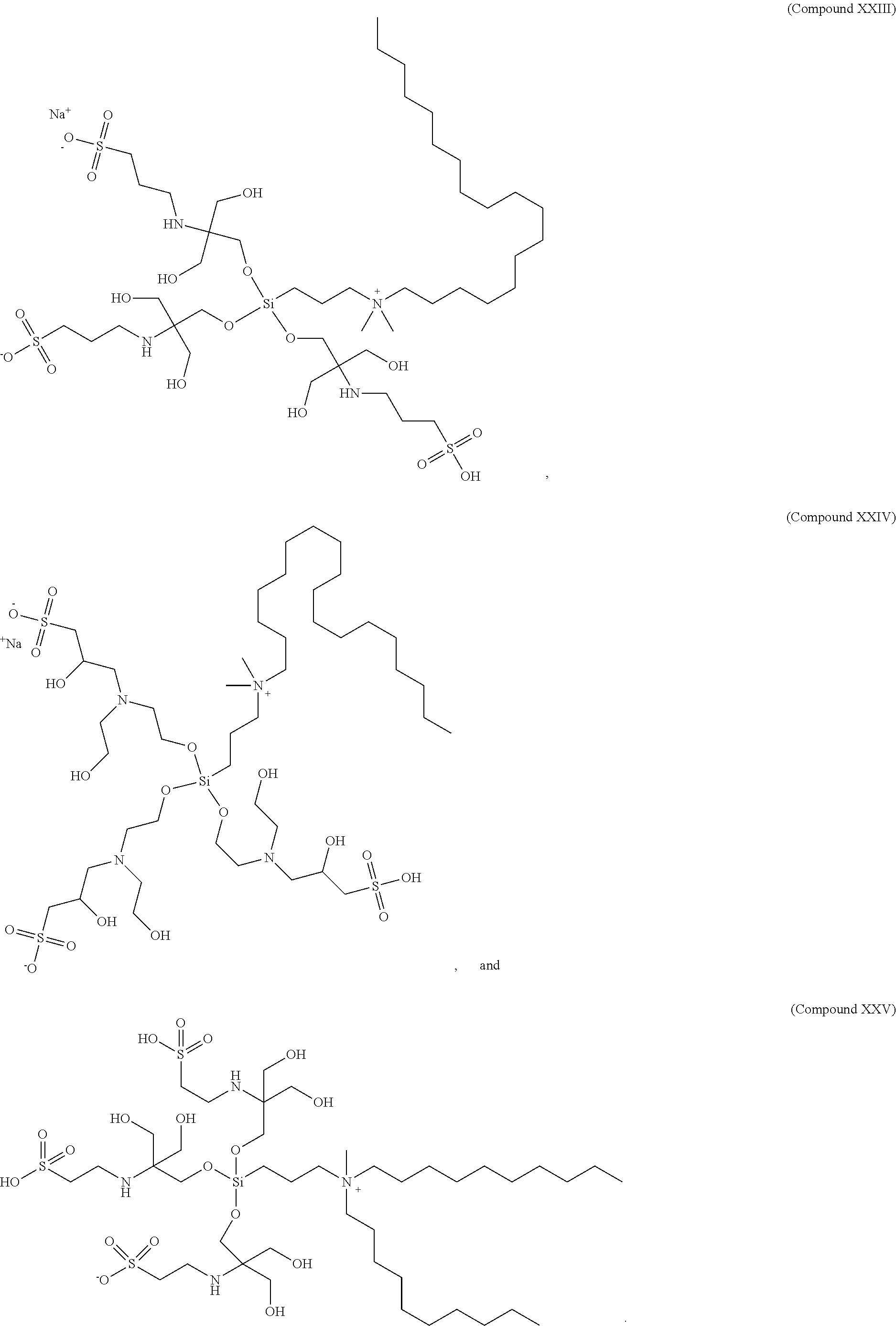

2. The aqueous hydrogel formulation of claim 1, wherein anion X⁻ is selected from methane sulfonate anion and

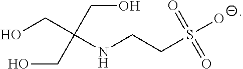 [SMILES: O=S(=O)([O-])CCNC(CO)(CO)CO]

3. The aqueous hydrogel formulation of claim 1 comprising:
a) between about 0.1% and 10% (weight/weight) (w/w) of a quaternary ammonium silane compound selected from the group consisting of Formula A-D and Compounds I-XXV;
b) between about 0.25% and about 3.0% (w/w) of the non-ionic gelling agent;
c) between about 0.025% and about 0.5% (w/w) of the non-ionic adhesion agent;
d) between about 0.5% and about 5.0% (w/w) of the non-ionic wetting agent;
e) optionally between about 0.5% and about 7.5% (w/w) of the non-ionic osmotic agent; and,
f) the aqueous solution.

4. The aqueous hydrogel formulation of claim 3 comprising:
a) a compound selected from the group consisting of Formula A-D and Compounds I-XXV, wherein the compound is present in the hydrogel formulation at a concentration that results in a trihydroxy-QAS cation present in the hydrogel at between about 0.1% and 5.0% (weight/weight) (w/w) when the compound is fully hydrolyzed, wherein the trihydroxy-QAS cation is of formula 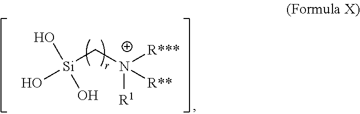
wherein
r is 1, 2, 3, or 4;
R¹ is C₆-C₂₂alkyl or C₆-C₂₂ alkanoyl;
R** and R*** are independently selected from the group consisting of:
i. C₁-C₄ alkyl; and,
ii. C₆-C₂₂alkyl or C₂-C₂₂alkanoyl;
b) between about 0.25% and about 3.0% (w/w) of a non-ionic gelling agent;
c) between about 0.025% and about 0.5% (w/w) of a non-ionic adhesion agent;
d) between about 0.5% and about 5.0% (w/w) of a non-ionic wetting agent; and
e) optionally between about 0.5% and about 7.5% (w/w) of a non-ionic osmotic agent.

5. The aqueous hydrogel formulation of claim 4, wherein the aqueous hydrogel formulation does not comprise the non-ionic osmotic agent.

6. The aqueous hydrogel formulation of claim 5, wherein the non-ionic gelling agent is present in a concentration from about 0.5% (w/w) to about 2.0% (w/w).

7. The aqueous hydrogel formulation of claim 6, wherein the non-ionic gelling agent is HPMC.

8. The aqueous hydrogel formulation of claim 5, wherein the non-ionic adhesion agent is present in the amount of between about 0.025% (w/w) and about 0.5% (w/w).

9. The aqueous hydrogel formulation of claim 5, wherein the non-ionic adhesion agent is a poloxamer.

10. The aqueous hydrogel formulation of claim 5, wherein the non-ionic wetting agent is present in a concentration of between about 1.0% (w/w) and 3.0% (w/w).

11. The aqueous hydrogel formulation of claim 5, wherein the non-ionic wetting agent is glycerin.

12. The aqueous hydrogel formulation of claim 5, wherein the aqueous solution is water.

13. The aqueous hydrogel formulation of claim 5, wherein the hydrogel comprises between about 0.25% and about 3% (w/w) trihydroxy-QAS cation of formula:

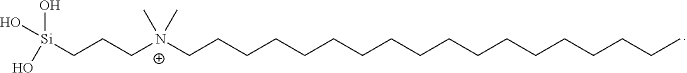 [SMILES: CCCCCCCCCCCCCCCCCC[N+](C)(C)CCC[Si](O)(O)O]

14. The aqueous hydrogel formulation of claim 4 comprising:
a. between about 0.3% and about 0.8% (w/w) of a trihydroxy-QAS cation of Formula X that is derived from the complete hydrolysis of a compound in the hydrogel selected from the group consisting of Formula A-D and Compounds I-XXV, or a combination thereof;
b. between about 0.8% and about 3.0% (w/w) of a non-ionic gelling agent;
c. between about 0.025% to and about 0.40% (w/w) of a non-ionic adhesion agent;
d. between about 0.5% and about 4.0% (w/w) of a non-ionic wetting agent; and
e. optionally, between about 0.05% and about 0.4% (w/w) of an additional agent.

15. The aqueous hydrogel formulation of claim 14 comprising:
a. between about 0.3% and about 0.6% (w/w) of a trihydroxy-QAS cation of Formula X that is derived from the complete hydrolysis of Compound XIX;
b. between about 0.8% and about 3.0% (w/w) of the non-ionic gelling agent, wherein the non-ionic gelling agent is HPMC;
c. between about 0.1% to and about 0.4% (w/w) of the non-ionic adhesion agent, wherein the non-ionic adhesion agent is Poloxamer 407; and
d. between about 0.5% and about 4.0% (w/w) of the non-ionic wetting agent, wherein the non-ionic wetting agent is glycerin.

16. The aqueous hydrogel formulation of claim 4 comprising:
a. between about 0.1% and 5.0% (weight/weight) (w/w) of a trihydroxy-QAS cation of Formula: 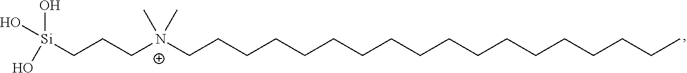 [SMILES: CCCCCCCCCCCCCCCCCC[N+](C)(C)CCC[Si](O)(O)O]
wherein the hydrogel formulation also comprises 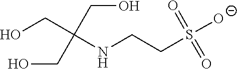 [SMILES: O=S(=O)([O-])CCNC(CO)(CO)CO]
as a counterion;
b. between about 0.25% and about 3.0% (w/w) of a non-ionic gelling agent;
c. between about 0.025% and about 0.5% (w/w) of a non-ionic adhesion agent;
d. between about 0.5% and about 5.0% (w/w) of a non-ionic wetting agent; and

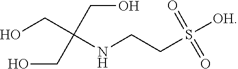 [SMILES: O=S(=O)(O)CCNC(CO)(CO)CO]

17. The aqueous hydrogel formulation of claim 16 comprising:
a. between about 0.1% and 5.0% (weight/weight) (w/w) of a trihydroxy-QAS cation of Formula: 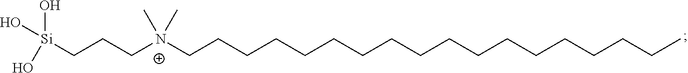 [SMILES: CCCCCCCCCCCCCCCCCC[N+](C)(C)CCC[Si](O)(O)O]
b. between about 0.25% and about 3.0% (w/w) of hydroxypropyl methylcellulose;
c. between about 0.025% and about 0.5% (w/w) of Poloxamer 407; and
d. between about 0.5% and about 5.0% (w/w) of glycerin.

18. The aqueous hydrogel formulation of claim 4 comprising:
a. between about 0.1% and 5.0% (weight/weight) (w/w) of a trihydroxy-QAS cation of Formula: 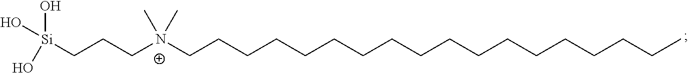 [SMILES: CCCCCCCCCCCCCCCCCC[N+](C)(C)CCC[Si](O)(O)O]
b. between about 0.25% and about 3.0% (w/w) of a non-ionic gelling agent;
c. between about 0.025% and about 0.5% (w/w) of a non-ionic adhesion agent; and
d. between about 0.5% and about 5.0% (w/w) of a non-ionic wetting agent.

19. The aqueous hydrogel formulation of claim 14 comprising:
a. between about 0.3% and about 0.6% (w/w) of a trihydroxy-QAS cation of Formula X that is derived from the complete hydrolysis of Compound VII;
b. between about 0.8% and about 3.0% (w/w) of the non-ionic gelling agent, wherein the non-ionic gelling agent is HPMC;
c. between about 0.1% to and about 0.4% (w/w) of the non-ionic adhesion agent, wherein the non-ionic adhesion agent is Poloxamer 407; and
d. between about 0.5% and about 4.0% (w/w) of the non-ionic wetting agent, wherein the non-ionic wetting agent is glycerin.

20. The aqueous hydrogel formulation of claim 1, wherein the compound is Compound VIII.

21. The aqueous hydrogel formulation of claim 1, wherein the compound is Compound XIX.

22. A method of aiding the cleaning of teeth in a subject comprising applying the topical formulation of claim 1 to the teeth of the subject.

23. The method of claim 22, wherein the subject is a human.

24. The method of claim 22, wherein the subject is a dog.

25. The method of claim 22, where the subject is a cat.
